(19) United States
(12) Patent Application Publication
(10) Pub. No.: US 2023/0042042 A1
(43) Pub. Date: Feb. 9, 2023

(54) UNNATURAL AMINO ACIDS AND USES THEREOF

(71) Applicant: Temple University-Of The Commonwealth System of Higher Education, Philadelphia, PA (US)

(72) Inventors: Christian Schafmeister, Philadelphia, PA (US); Justin Northrup, Philadelphia, PA (US)

(21) Appl. No.: 17/785,184

(22) PCT Filed: Dec. 17, 2020
(86) PCT No.: PCT/US2020/065608

§ 371 Date: Jun. 14, 2022

Related U.S. Application Data
(60) Provisional application No. 62/948,954, filed on Dec. 17, 2019.

(51) Int. Cl. C07D 487/10; C07D 487/22; C07D 487/20; A61K 31/4166

(52) U.S. Cl. CPC C07D 487/10; C07D 487/22; C07D 487/20; A61K 31/4166

(57) ABSTRACT

This invention provides unnatural amino acids as well as amino acid sequences, foldamers, macrocycles, and formulations comprising said unnatural amino acids. In one aspect of the invention, the unnatural amino acids or the formulation thereof are used to mimic natural amino acids or the formulations thereof in a subject in need thereof.

### CROSS-REFERENCE TO RELATED APPLICATIONS

This application claims priority to and the benefit of U.S. Provisional Application No. 62/948,954, filed Dec. 17, 2019, the disclosure of which is incorporated herein by reference in its entirety.

### STATEMENT REGARDING FEDERALLY SPONSORED RESEARCH OR DEVELOPMENT

This invention was made with government support under 1R01GM123296 awarded by the National Institute of Health, 1625061 awarded by National Science Foundation, W911NF-16-2-0189 awarded by US Army Research Laboratory, HDTRA1-16-1-0047 awarded by the Department of the Defense, Defense Threat Reduction Agency, and R01GM114358 of the NIH to RHGB awarded by the National Institute of General Medical Sciences. The government has certain rights in the invention.

### BACKGROUND OF THE INVENTION

Mimicking amino acids and peptides with unnatural amino acids or peptidomimetics has been a vital area of research for many decades. Significant effort has been applied to mimic the structural and functional diversity of natural proteins and peptides using peptidomimetics, from beta-amino acids (Cheng R P et al., 2001, Chem. Rev., 101:3219; Murray J K et al., 2005, Org. Lett., 7:1517) and stapled peptides (Blackwell H E et al., 1998, Angew. Chem. Int. Ed., 37:3281; Schafmeister C E et al., 2000, J. Am. Chem. Soc., 122:5891; Verdine G L et al., 2012, Stapled Peptides for Intracellular Drug Targets, 1st ed.; Elsevier Inc., Vol. 503, pp 3-33; Bernal F et al., 2007, J. Am. Chem. Soc., 129:2456) to peptoids (Zuckermann R et al., 2009, Curr. Opin. Mol. Ther., 11:299; Zuckermann R N et al., 2010, Biopolymers, 96:545; Sun J et al., 2013, ACS Nano, 7:4715; Zuckermann R N et al., 1992, J. Am. Chem. Soc., 114:10646.) and spiroligomers (Cheong J E et al., 2016, Tetrahedron Lett., 57:4882; Schafmeister C E et al., 2008, Accounts Chem. Res., 41:1387). One particular amino acid of interest is proline, an amino acid that is prevalent across a wide array of natural and synthetic structures (Krapcho J et al., 1998, J. Medicin. Chem., 31:1148; Koskinen A M P et al., 1989, J. Org. Chem., 54:1859; Gruttadauria M et al., 2008, Chem. Soc. Rev., 37:1666; List B et al., 2002, Tetrahedron, 58:5573; Panday S K et al., 2011, Tetrahedron-Asymmetr., 22:1817), with nonproteinogenic proline being used for conformationally rigid peptides, angiotensin converting enzyme inhibitors (Krapcho J et al., 1998, J. Medicin. Chem., 31:1148), and asymmetric synthesis (Gruttadauria M et al., 2008, Chem. Soc. Rev., 37:1666; List B et al., 2002, Tetrahedron, 58:5573; Panday S K et al., 2011, Tetrahedron-Asymmetr., 22:1817) among other applications (Panday S K et al., 2011, Tetrahedron-Asymmetr., 22:1817). Many nonproteinogenic prolines are functionalized at the 4-position of the pyrrolidine ring, synthetically accessed from L-trans-4-hydroxyproline (Koskinen A M P et al., 1989, J. Org. Chem., 54:1859; Panday S K et al., 2011, Tetrahedron-Asymmetr., 22:1817).

Thus, there is a need in the art for compounds, compositions, and methods comprising unnatural amino acids that mimic natural amino acids and/or provide functionalities (i.e., functional groups) that are not found in the natural amino acids. The present invention addresses this unmet need in the art.

### SUMMARY OF THE INVENTION

In one aspect, the present invention provides a compound or salt thereof having the structure of Formula (I)

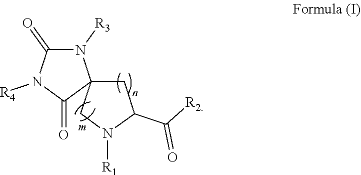

In some embodiments, R₁ is H or a protecting group.

In some embodiments, each R₂, R₃, and R₄ is independently H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted —Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural amino acid, unnatural amino acid, or

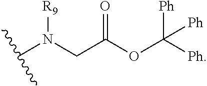

In some embodiments, Y is C, N, O, S, or P. In some embodiments, each R₅, R₆, and R₇ is independently H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, or sulfoxy. In some embodiments, o is an integer represented by 0, 1, or 2. In some embodiments, p is an integer represented by 0, 1, or 2.

In some embodiments, m is an integer represented by 1 or 2. In some embodiments, n is an integer represented by 1 or 2.

In various embodiments, the compound is an unnatural amino acid.

In various embodiments, the protecting group is a carbonyl protecting group, carbamate protecting group, sulfonamide protecting group, trityl protecting group, 1-(4,4-dimethyl-2,6-dioxocyclohex-1-ylidene)-3-ethyl (Dde) protecting group, or 1-(4,4-dimethyl-2,6-dioxocyclohex-1-ylidene)-3-methylbutyl (ivDde) protecting group.

In some embodiments, the carbonyl protecting group is a methoxycarbonyl protecting group, tert-butoxycarbonyl protecting group (BOC group), benzyloxycarbonyl protecting group,

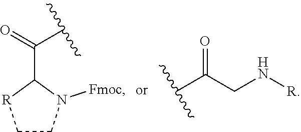

In some embodiments, the methoxycarbonyl protecting group is 9-fluorenyl methoxycarbonyl (Fmoc).

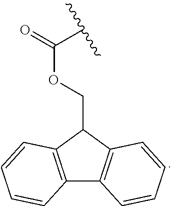 [SMILES: *C(=O)OCC1c2ccccc2-c2ccccc21]

In one embodiment, R₁ is a protecting group; R₂ is hydroxyl, hydroxyalkyl, hydroxyalkenyl, hydroxyalkynyl, or hydroxyaryl; m is an integer represented by 1; and n is an integer represented by 1. In another embodiment, R₁ is a protecting group; R₂ is hydroxyl, hydroxyalkyl, hydroxyalkenyl, hydroxyalkynyl, or hydroxyaryl; m is an integer represented by 2; and n is an integer represented by 1. In yet another embodiment, R₁ is a protecting group; R₂ is hydroxyl, hydroxyalkyl, hydroxyalkenyl, hydroxyalkynyl, or hydroxyaryl; m is an integer represented by 1; and n is an integer represented by 2.

In some embodiments, the compound comprises proline.

In various embodiments, the compound is

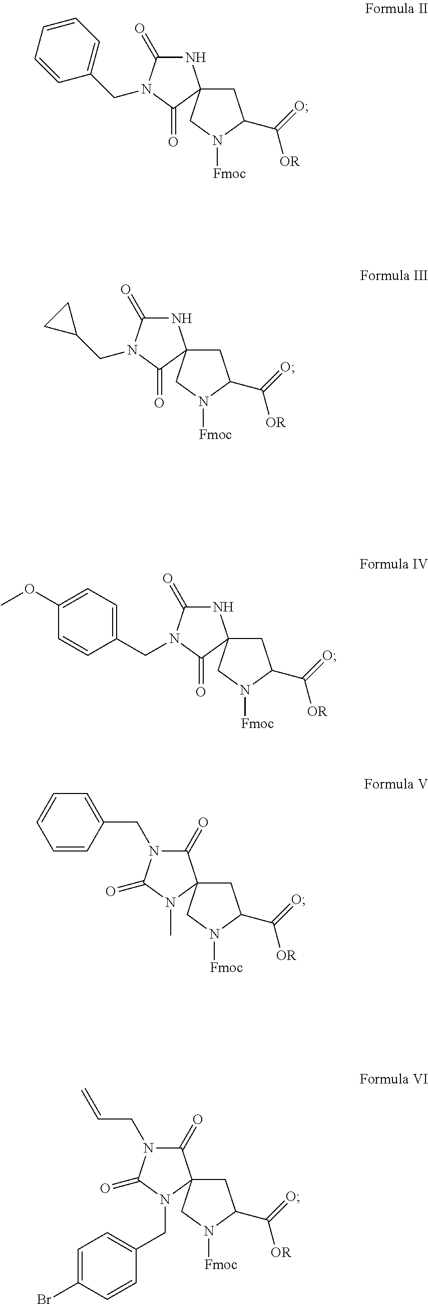

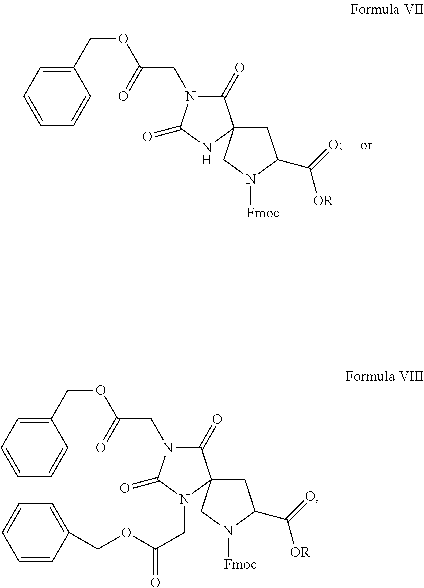

In some embodiments, R is H, alkyl, or a protecting group.

In various embodiments, the compound comprises at least two stereocenters. Thus, in some embodiments, the compound is

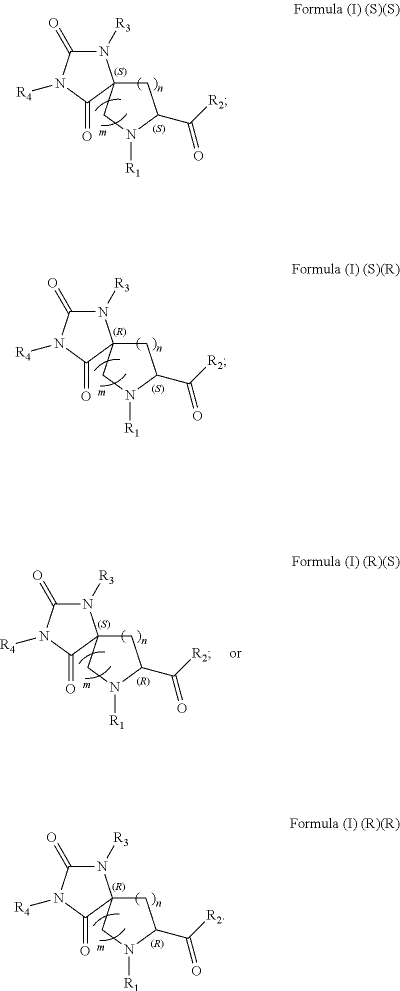

In some embodiments, the compound is

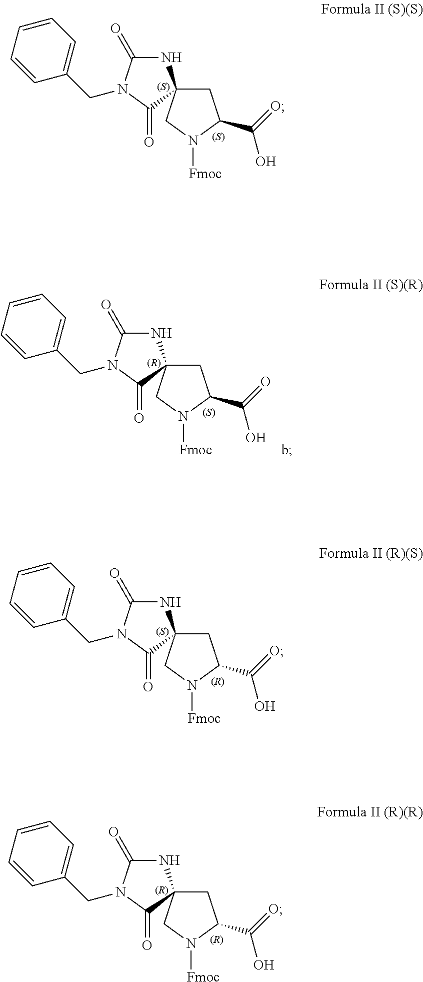

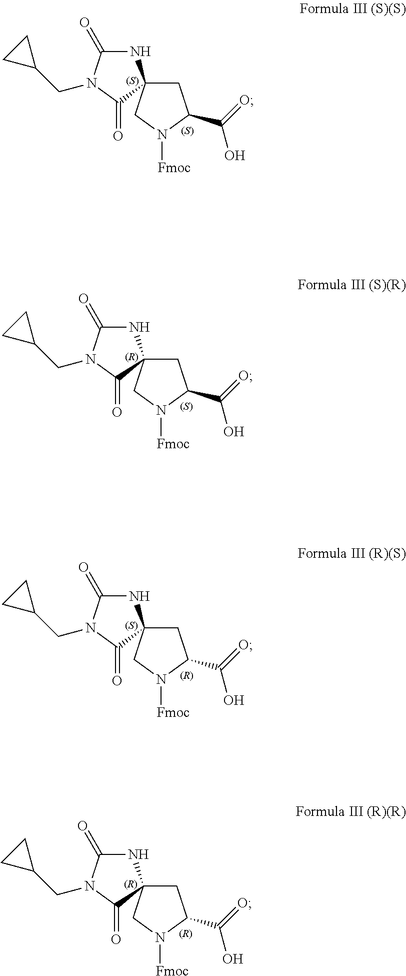

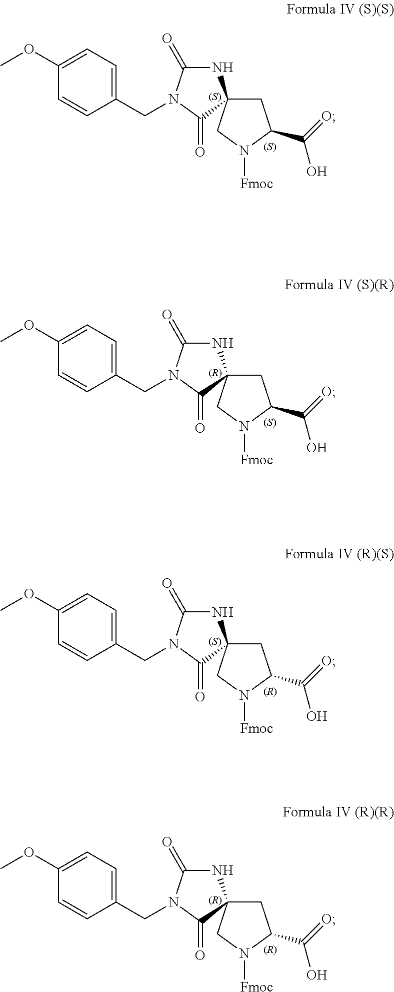

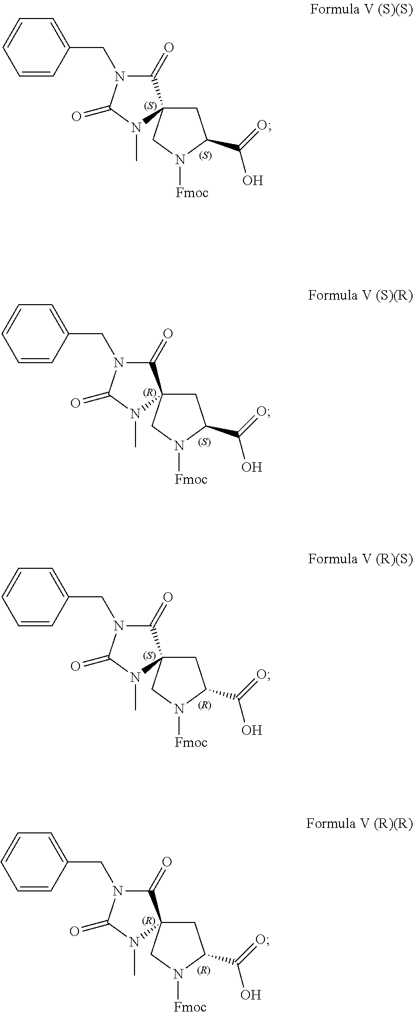

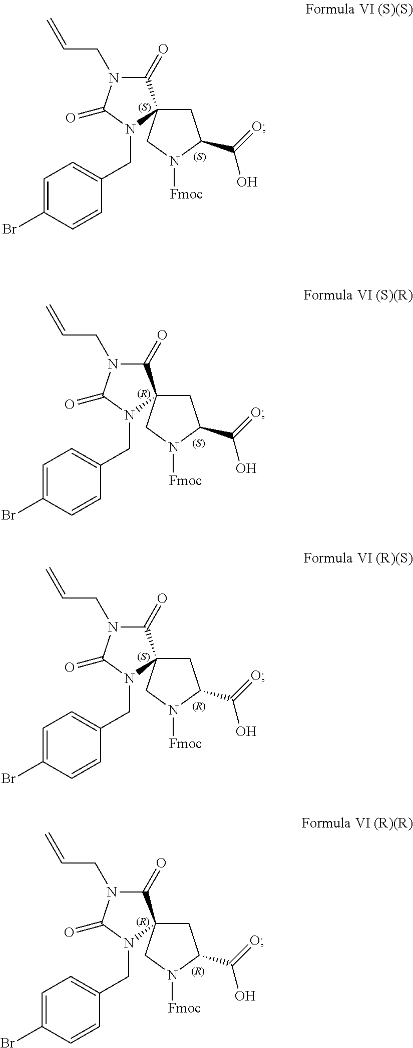

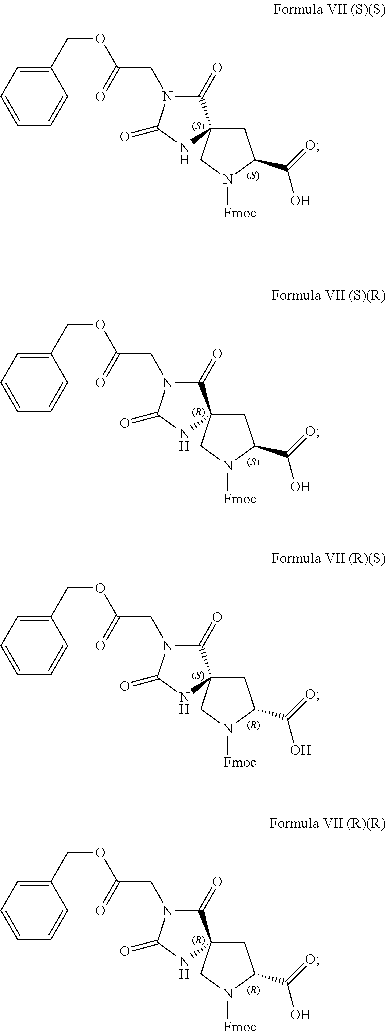

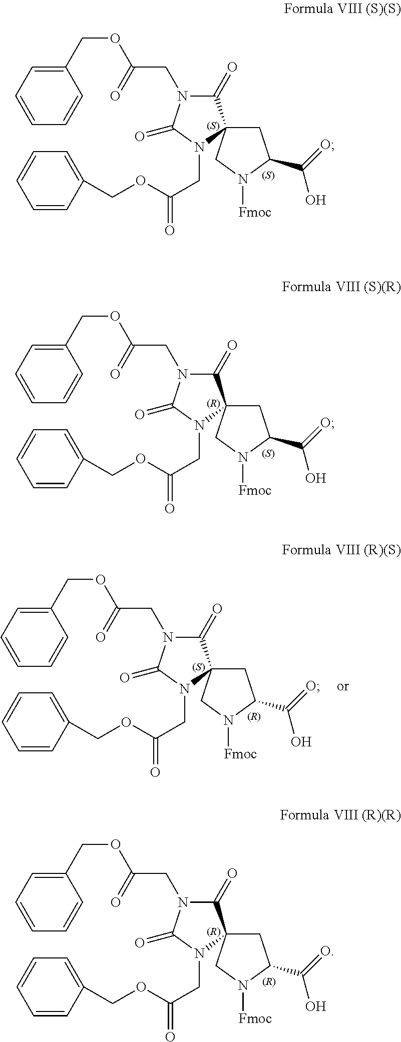

In one aspect, the present invention also discloses an amino acid sequence comprising one or more compounds described herein. In some embodiments, the amino acid sequence is a peptide or a fragment thereof, polypeptide or a fragment thereof, or protein or a fragment thereof.

In another aspect, the present invention discloses a foldamer comprising one or more compounds described herein. In some embodiments, the foldamer is a peptidomimetic foldamer, peptide, bispeptide, β-peptide, γ-peptide, δ-peptide, nucleotidomimetic foldamer, abiotic foldamer, peptoid, aedamer, aromatic oligamide foldamer, spiroligomer, arylamine foldamer, and chiral oligomers of pentenoic amides (COPAs).

In another aspect, the present invention discloses a macrocycle comprising one or more compounds of described herein. In some embodiments, the macrocycle has the structure of Formula (IX)

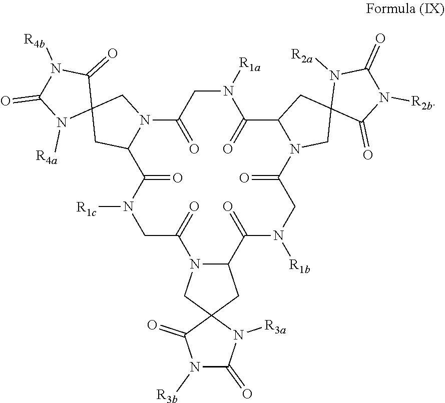

In some embodiments, each R₁ₐ, R_{1b}, R_{1c}, R₂ₐ, R_{2b}, R₃ₐ, R_{3b}, R₄ₐ, and R_{4b} is independently H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted —Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural amino acid, unnatural amino acid, or

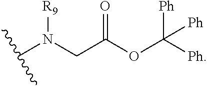

In various embodiments, the macrocycle is

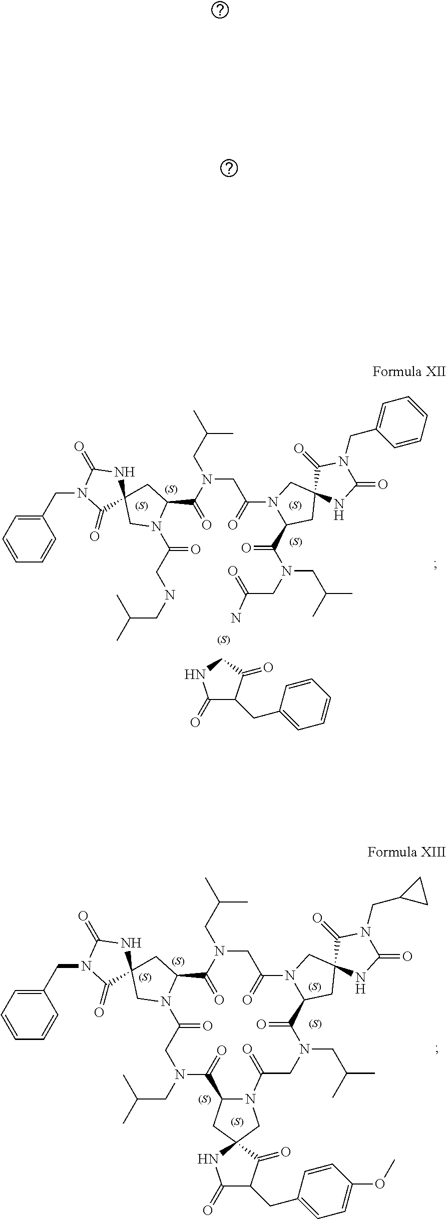

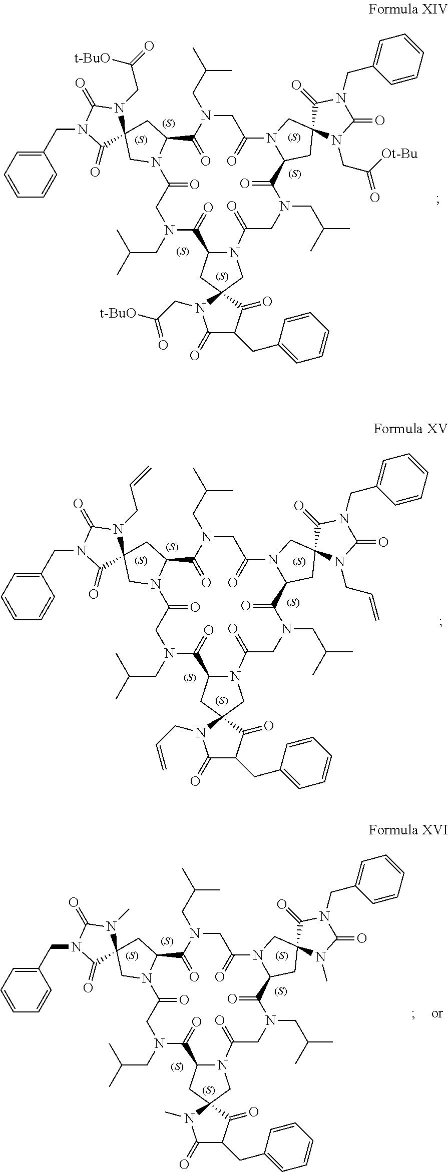

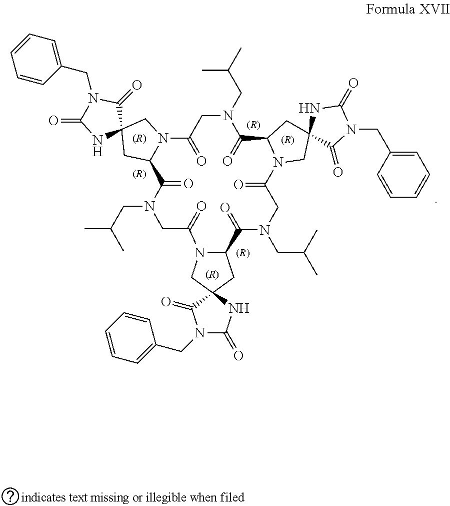

In various embodiments, the macrocycle further comprises at least one metal ion. In one embodiment, the metal ion is a metal cation. In some embodiments, the metal cation is a Li⁺, Na⁺, or K⁺.

In one aspect, the present invention discloses a composition comprising one or more compounds described herein.

In another aspect, the present invention discloses a method of mimicking a natural amino acid in a subject in need thereof, the method comprising administering to the subject a therapeutically effective amount of one or more compound of the present invention or a composition thereof. In some embodiments, the compound of the present invention mimics the natural amino acid. In some embodiments, the compound of the present invention mimics the function of the natural amino acid. In some embodiments, the compound of the present invention mimics the structure of the natural amino acid.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts the synthesis of Q-prolines (Q-Pro) 1-12 from the stereochemically pure starting material 1. **Q-Pro 6 and Q-Pro 12 were synthesized together and separated via Flash Chromatography, yield represents combined yields.

FIG. 2, comprising FIG. 2A through FIG. 2C, depicts exemplary elution chromatograms of Q-Pro 5, Q-Pro 6, and Q-Pro 12 obtained using reversed-phase flash chromatography. FIG. 2A depicts exemplary elution chromatogram of Q-Pro 5 reaction mixture obtained using reversed-phase flash chromatography. FIG. 2B depicts exemplary elution chromatogram of Q-Pro 6 and Q-Pro 12 reaction mixture obtained using reversed-phase flash chromatography. FIG. 2C depicts exemplary elution chromatogram of Q-Pro 5 obtained using reversed-phase high-performance liquid chromatography (HPLC). This HPLC chromatogram is exemplary of the high purity of Q-Pro residues purified via reversed-phase flash chromatography.

FIG. 3 depicts coupling protogenic residues to a solid supported Q-proline residue with varied times, temperatures, equivalents of protogenic residues, and whether double couplings were utilized. Trials 27-30 were performed with a microwave reactor; time 3-2 denotes a 3 minute ramp to temp and a 2 minute hold at temp. Temp 60* for trials 29-30 signifies use of compressed air cooling during the hold.

FIG. 4 depicts coupling proteogenic residues to a solid supported Q-proline residue at varied temperatures. In order to calculate the approximate % completion, after reacting with the next Fmoc amino acid, 10 equiv of Fmoc-OSu was reacted with the resin to Fmoc protect any free amine still present on resin. The resin was cleaved, and the ratio of product to starting material calculated from integrated UV chromatograms.

FIG. 5, comprising FIGS. 5A and 5B, depicts exemplary HPLC chromatogram result and three-dimensional CANDO model of compound Q-Pro 5 (Fmoc deprotected). FIG. 5A depicts exemplary HPLC chromatogram result demonstrating incomplete coupling of Trials 11-13 from FIG. 3, as indicated by the large amount of starting material, compound 4, versus product, compound 5. FIG. 5B depicts three-dimensional CANDO model of compound Q-Pro 5 (Fmoc deprotected) with solvent-excluded surface (image generated with Chimera).

FIG. 6, comprising FIG. 6A and FIG. 6B, depicts exemplary HPLC chromatogram results of successful proline-to-Q-Proline coupling reactions. FIG. 6A depicts exemplary HPLC chromatogram result of a successful proline-to-Q-Proline coupling reaction performed in a conventional oven reactor (Conversion of compound 4 to Compound 5). FIG. 6B depicts exemplary HPLC chromatogram result of a successful proline-to-Q-Proline coupling reaction performed in a microwave synthesis reactor (Conversion of compound 4 to Compound 5).

FIG. 7 depicts a solid phase synthesis of Q-proline macrocycles on 2-Cl-Trt-Cl resin. (a) depicts the step comprising of i. bromoacetic acid, DCM, DIPEA; ii. R₃—NH2, DMF; (b) depicts the step comprising of Q-Pro, HATU, DMF, DIPEA; (c) depicts the step comprising of i. 20% Piperdine/DMF; ii. bromoacetic acid, DIC, DMF; iii. R₃—NH2, DMF; (d) depicts the step comprising of i. 20% Piperdine/DMF; ii. 30% HFIP/DCM; (e) depicts the step comprising of DMF, PyAOP, DIPEA. (f) depict the step comprising of alkenyl or benzyl bromide, DMF, K₂CO₃.

FIG. 8 depicts the chemical structure of various monoalkylated Q-proline macrocycles and the corresponding percent yields.

FIG. 9, comprising FIG. 9A and FIG. 9B, depicts the structure and the corresponding ¹H NMR spectra of a Q-Pro Macrocycle (QPM-3). FIG. 9A depicts structure of QPM-3. FIG. 9B depicts ¹H NMR of QPM-3 with 4.0 equiv. of various metal triflates.

FIG. 10 depicts exemplary ¹NMR spectra that indicate that magnesium, calcium, scandium, copper, and europium triflates did not provide symmetric structures of the macrocycles.

FIG. 11, comprising FIG. 11A through FIG. 11D, depicts exemplary ¹H NMR spectra and structure of various macrocycle-triflate complexes. FIG. 11A depicts the chemical structure of QPM-3. FIG. 11B depicts exemplary ¹H NMR spectra of QPM-3 in the presence and absence of various metal triflates. FIG. 11C depicts exemplary ¹H NMR spectra of QPM-3 in the presence of various equiv. of potassium triflate. FIG. 11D depicts exemplary ¹H NMR spectra of QPM-3 with potassium triflate after various times.

FIG. 12 depicts an overlay of exemplary COSY spectra of QPM-1, QPM-2, and QPM-3 showing remarkable similarities in shifts for the α (4.98-5.02 ppm), R (2.10-2.16 ppm and 2.70-2.74 ppm), and 6 (3.83-3.87 ppm and 3.96-4.00 ppm) protons of the proline ring.

FIG. 13 depicts an overlay of exemplary ROESY spectra of QPM-3 and QPM-5 showing a marked difference in the proline envelope conformation for the dialkylated macrocycles.

FIG. 14, comprising FIG. 14A and FIG. 14B, depicts the racemic crystal structure of QPM-3 (grey) and its enantiomer (light-blue) with side chains shown in orange (solvent omitted for clarity). FIG. 14A depicts a side view across the macrocycle. FIG. 14B depicts top down view looking through the macrocyclic core.

FIG. 15 depicts schematic representation of exemplary Q-Pro macrocycle structures before after the addition of potassium ion.

FIG. 16, comprising FIG. 16A and FIG. 16B, depicts exemplary thermal ellipsoid plots of the S-enantiomer and the R-enantiomer of QPM-3, each displayed with the nearest ethyl acetate solvate molecule, obtained using single-crystal X-ray crystallography. Ellipsoids set at 30% probability and hydrogen atoms omitted for clarity. FIG. 16A depicts an exemplary thermal ellipsoid plot of the S-enantiomer of QPM-3 displayed with the nearest ethyl acetate solvate molecule. FIG. 16B depicts an exemplary thermal ellipsoid plot of the R-enantiomer of QPM-3 displayed with the nearest ethyl acetate solvate molecule.

### DETAILED DESCRIPTION

The present invention provides novel unnatural amino acids as well as amino acid sequences, foldamers, macrocycles, and compositions comprising said unnatural amino acids. In one aspect of the invention, the unnatural amino acid mimics a natural amino acid. In one aspect of the invention, the unnatural amino acid comprises a functionality, such as a functional group, that is not found in a natural amino acid. In one aspect of the invention, the unnatural amino acid sequence mimics a natural amino acid sequence. In one aspect of the invention, the unnatural amino acid sequence comprises a functionality, such as a functional group, that is not found in a natural amino acid sequence. Thus, in some aspects, the present invention also relates to the method of mimicking natural amino acids using the compounds or the compositions thereof of the present invention.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, such as an amount, a temporal duration, and the like, is meant to encompass variations of 20%, ±10%, ±5%, ±1%, or 0.1% from the specified value, as such variations are appropriate to perform the disclosed methods.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂-)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to aryl groups which contain at least one heteroatom selected from N, O, Si, P, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl. Additionally, protecting groups include, but are not limited to, photolabile groups, such as Nvoc and MeNvoc, and other protecting groups known in the art. Other protecting groups are described in Greene and Wuts, Protective Groups in Organic Synthesis, 3rd Ed., John Wiley & Sons, New York, N.Y., 1999, which is incorporated herein by reference in its entirety.

As used herein, the term “macrocycle” means a molecule containing a 12-member ring or larger.

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “natural amino acid”, “naturally encoded amino acid”, “naturally occurring amino acid”, and “genetically encoded amino acid” refer to an amino acid that is one of the twenty common amino acids or pyrolysine or selenocysteine. The term “natural amino acid” includes, but is not limited to, proteinogenic amino acids.

A “non-natural amino acid” refers to an amino acid that is not one of the twenty common amino acids or pyrolysine or selenocysteine. Other terms that may be used synonymously with the term “non-natural amino acid” is “non-naturally encoded amino acid,” “unnatural amino acid,” “non-naturally-occurring amino acid,” “non-genetically encoded amino acid”, and variously hyphenated and non-hyphenated versions thereof. The term “non-natural amino acid” includes, but is not limited to, amino acids which occur naturally by modification of a naturally encoded amino acid (including but not limited to, the common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, nonproteinogenic amino acids and amino acids, which do not occur naturally and may be obtained synthetically (e.g., Q-proline-based amino acids) or may be obtained by modification of non-natural amino acids.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides”. The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “recombinant polypeptide” as used herein is defined as a polypeptide produced by using recombinant DNA or RNA methods.

The term “DNA” as used herein is defined as deoxyribonucleic acid.

The term “RNA” as used herein is defined as ribonucleic acid.

The term “recombinant DNA” as used herein is defined as DNA produced by joining pieces of DNA from different sources.

The term “recombinant RNA” as used herein is defined as RNA produced by joining pieces of RNA from different sources.

As used herein, the term “identical” refers to two or more sequences or subsequences which are the same.

In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring, such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

As used herein, “fragment” is defined as at least a portion of the variable region of the immunoglobulin molecule which binds to its target, i.e. the antigen binding region. Some of the constant region of the immunoglobulin may be included.

As used herein, the term “linkage” refers to bonds or chemical moiety formed from a chemical reaction between the functional group of a linker and another molecule. Such bonds may include, but are not limited to, covalent linkages and non-covalent bonds, while such chemical moieties may include, but are not limited to, esters, carbonates, imines phosphate esters, hydrazones, acetals, orthoesters, peptide linkages, and oligonucleotide linkages. Hydrolytically stable linkages means that the linkages are substantially stable in water and do not react with water at useful pH values, including but not limited to, under physiological conditions for an extended period of time, perhaps even indefinitely. Hydrolytically unstable or degradable linkages means that the linkages are degradable in water or in aqueous solutions, including for example, blood. Enzymatically unstable or degradable linkages means that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The terms “effective amount” and “pharmaceutically effective amount” refer to a sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation. An “effective amount” or “therapeutically effective amount” of a compound is that amount of compound, which is sufficient to provide a beneficial effect to the subject to which the compound is administered.

As used herein, “pharmaceutically-acceptable” means that drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio.

The terms “patient”, “subject”, “individual”, and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, and mice and humans.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, “antigen-binding domain” means that part of the antibody, recombinant molecule, the fusion protein, or the immunoconjugate of the invention which recognizes the target or portions thereof.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause one decrease in the subject's state of health.

A disease or disorder is “alleviated” if the severity of a sign or symptom of the disease or disorder, the frequency with which such a sign or symptom is experienced by a subject, or both, is reduced.

A “therapeutic treatment” is a treatment administered to a subject who exhibits signs of disease or disorder, for the purpose of diminishing or eliminating those signs.

As used herein, “treating a disease or disorder” means reducing the frequency and/or severity of a sign and/or symptom of the disease or disorder is experienced by a subject.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

By the term “specifically binds,” as used herein, is meant a molecule, such as an antibody, which recognizes and binds to another molecule or feature, but does not substantially recognize or bind other molecules or features in a sample.

As used herein, “fused” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct fusion involves one molecule attached to the molecule of interest. Indirect fusion involves one molecule attached to another molecule which in turn is attached directly or indirectly to the molecule of interest.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

This invention relates, in part, to novel unnatural amino acids and compositions thereof. In one aspect of the invention, the unnatural amino acid mimics a natural amino acid. In one aspect of the invention, the unnatural amino acid comprises a functionality, such as a functional group, that is not found in a natural amino acid. In various aspects, the present invention also provides amino acid sequences, foldamers, and/or macrocycles comprising one or more unnatural amino acids of the present invention. In some aspects, the present invention also relates to the method of mimicking natural amino acids using the compounds or the compositions thereof of the present invention.

### Unnatural Amino Acids

The present invention relates, in part, to an unnatural amino acid. In various aspects, the unnatural amino acid is a compound or salt thereof having the structure of Formula (I)

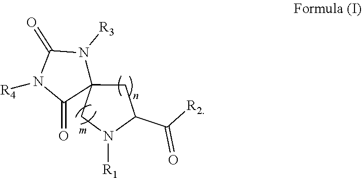

In some embodiments, R₁ is H or a protecting group. In various embodiments, the protecting group is a carbonyl protecting group, carbamate protecting group, sulfonamide protecting group, trityl protecting group, 1-(4,4-dimethyl-2,6-dioxocyclohex-1-ylidene)-3-ethyl (Dde) protecting group, or 1-(4,4-dimethyl-2,6-dioxocyclohex-1-ylidene)-3-methylbutyl (ivDde) protecting group.

In some embodiments, the carbonyl protecting group is a methoxycarbonyl protecting group, tert-butoxycarbonyl protecting group (BOC group), benzyloxycarbonyl protecting group,

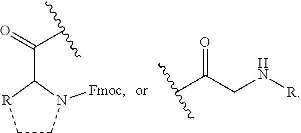

In one embodiment, the methoxycarbonyl protecting group is 9-fluorenyl methoxycarbonyl (Fmoc)

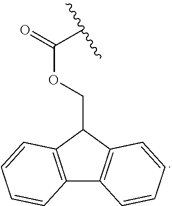 [SMILES: *C(=O)OCC1c2ccccc2-c2ccccc21]

In one embodiment, the carbamate protecting group is a para-nitrobenzyl carbamate protecting group (pNZ group). In one embodiment, the carbamate protecting group is an allyloxycarbonyl (alloc) protecting group. In one embodiment, the protecting group is the 2-nitrobenzenesulfonamide (Fukuyama sulfonamide) protecting group.

In some embodiments, R₂, R₃, or R₄ is H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted —Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural amino acid, unnatural amino acid, or

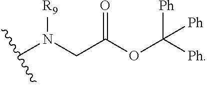

In various embodiments, Y is C, N, O, S, or P.

In some embodiments, o is an integer represented by 0, 1, 2, or 3. In some embodiments, p is an integer represented by 0, 1, 2, or 3.

In some embodiments, R₅, R₆, or R₇ is H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, or sulfoxy.

In some embodiments, m is an integer represented by 1 or 2. In some embodiments, n is an integer represented by 1 or 2. For example, in some embodiments, m is an integer represented by 1 and n is an integer represented by 1. In some embodiments, m is an integer represented by 1 and n is an integer represented by 2. In some embodiments, m is an integer represented by 2 and n is an integer represented by 1.

In various aspects of the invention, the unnatural amino acid is a monofunctional amino acid. In various aspects of the invention, the unnatural amino acid is a bifunctional amino acid. In some embodiments, the unnatural amino acid having the structure of Formula (I) is a monofunctional amino acid. In some embodiments, the unnatural amino acid having the structure of Formula (I) is a bifunctional amino acid.

For example, in one embodiment, the unnatural amino acid is a compound having the structure of Formula (I), wherein R₁ is a protecting group; R₂ is hydroxyl, hydroxyalkyl, hydroxyalkenyl, hydroxyalkynyl, or hydroxyaryl; m is an integer represented by 1; and n is an integer represented by 1.

In various embodiment, the unnatural amino acid comprises a proline molecule. In some embodiments, the unnatural amino acid having the structure of Formula (I) comprises a proline molecule.

In some embodiments, the unnatural amino acid is a compound having the structure of

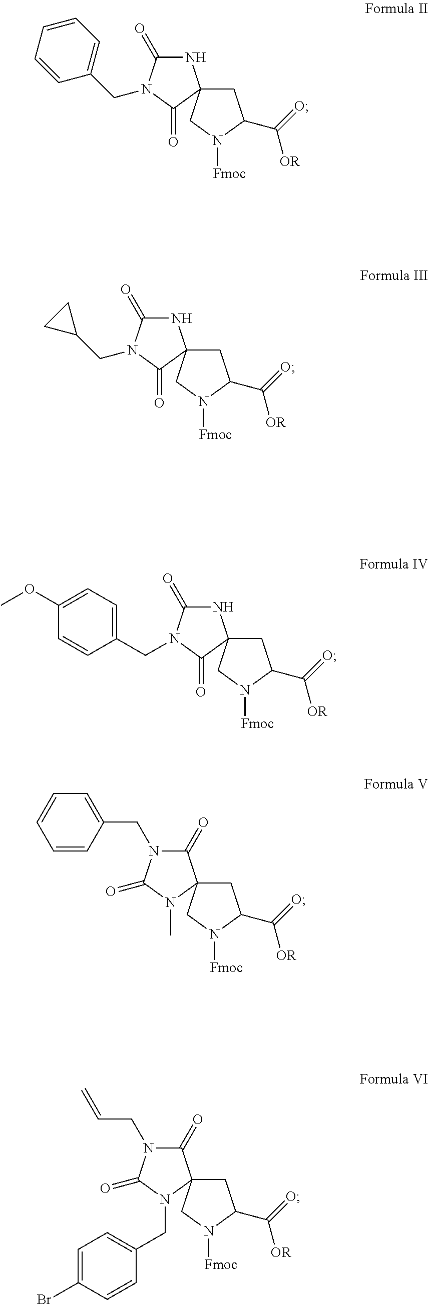

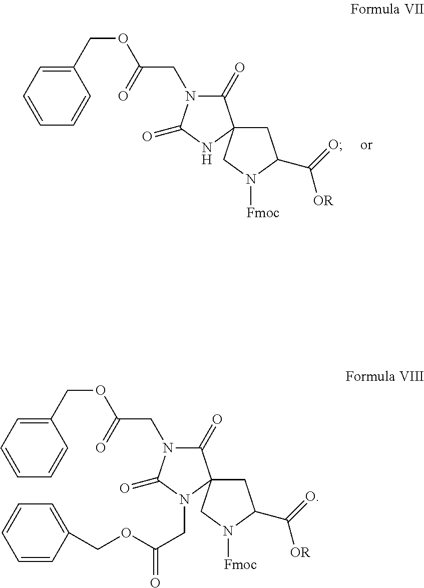

In some embodiments, R is H, alkyl, aryl, or a protecting group.

In various aspects of the invention, the unnatural amino acid comprises two or more stereocenters. In some embodiments, the unnatural amino acid having the structure of Formula (I) comprises two or more stereocenters. In some embodiments, the unnatural amino acid having the structure of Formula (II) comprises two or more stereocenters. In some embodiments, the unnatural amino acid having the structure of Formula (III) comprises two or more stereocenters. In some embodiments, the unnatural amino acid having the structure of Formula (IV) comprises two or more stereocenters. In some embodiments, the unnatural amino acid having the structure of Formula (V) comprises two or more stereocenters. In some embodiments, the unnatural amino acid having the structure of Formula (VI) comprises two or more stereocenters. In some embodiments, the unnatural amino acid having the structure of Formula (VII) comprises two or more stereocenters. In some embodiments, the unnatural amino acid having the structure of Formula (VIII) comprises two or more stereocenters.

Thus, in various aspects of the invention, the unnatural amino acid comprises at least two stereocenters. In some embodiments, the unnatural amino acid having the structure of Formula (I) comprises at least two stereocenters.

In some embodiments, the unnatural amino acid is a compound having the structure of

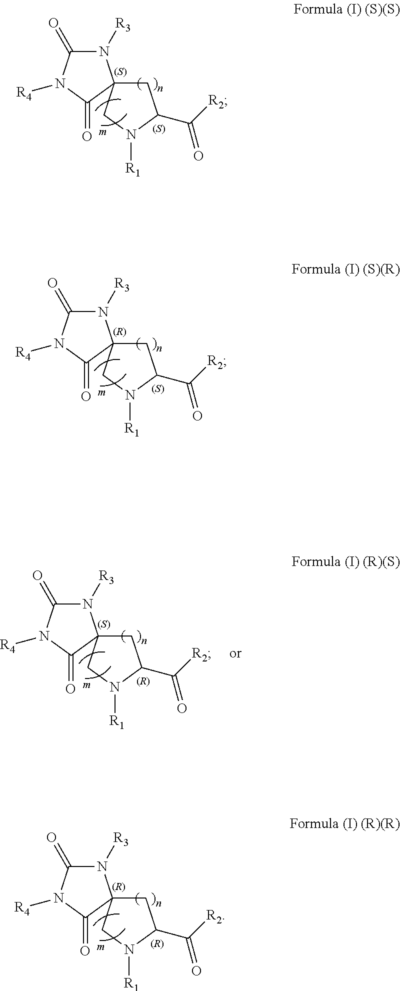

In various embodiments, the unnatural amino acid is a compound having the structure of

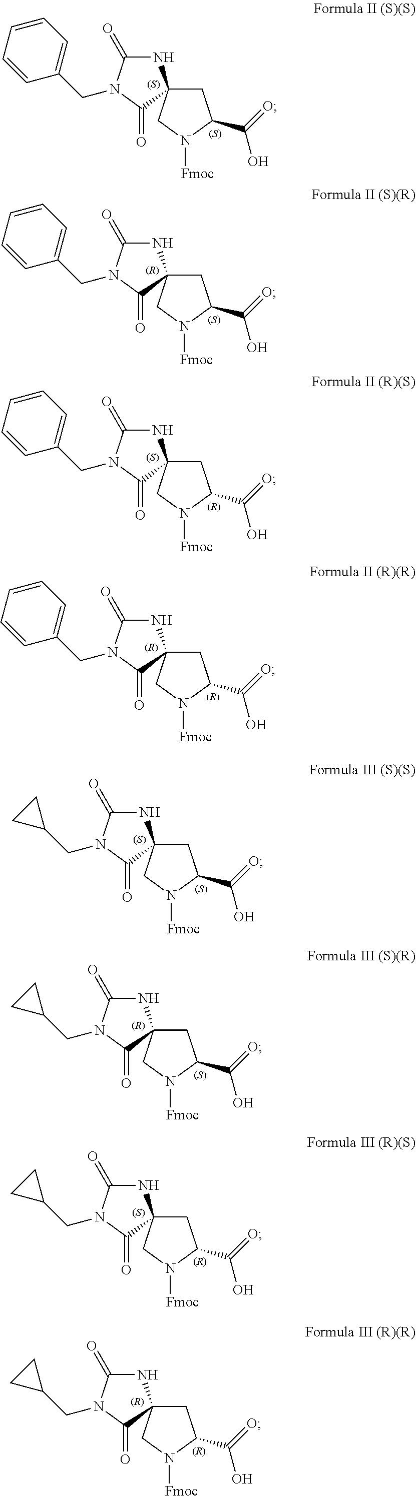

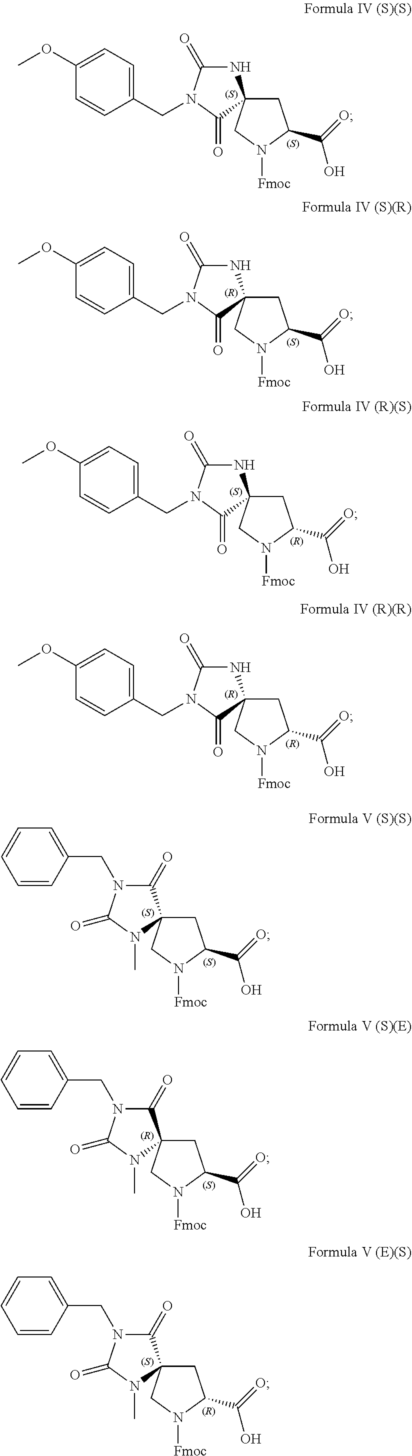

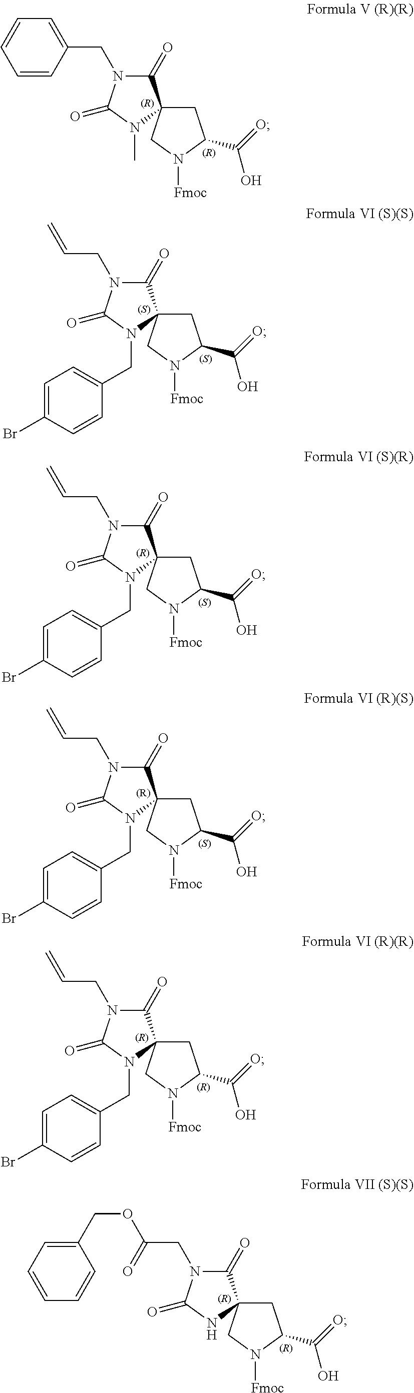

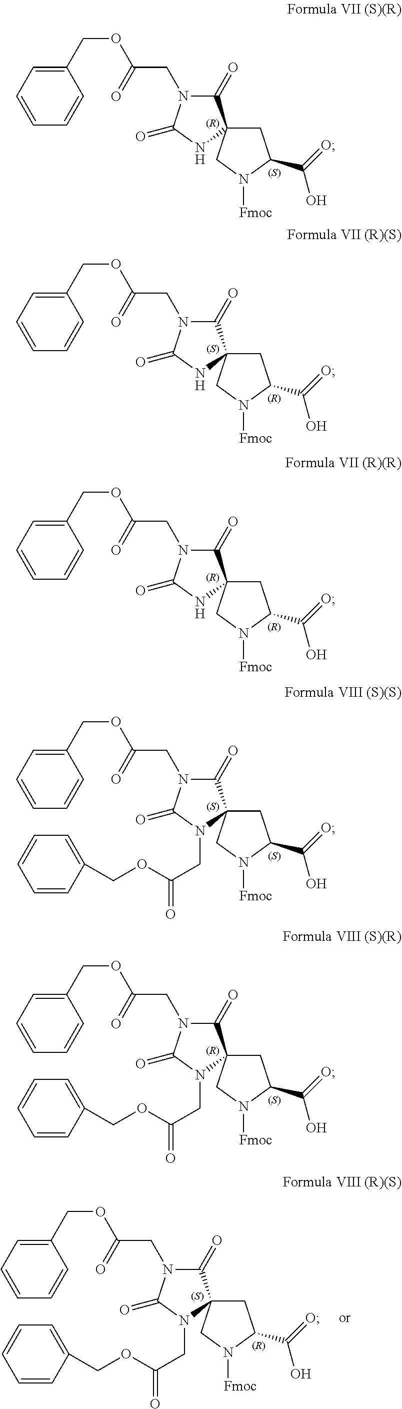

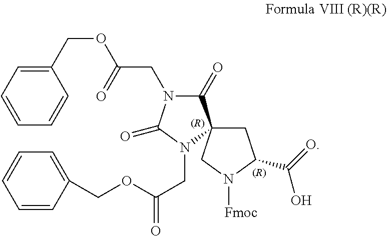

In various embodiments, the non-natural amino acids have properties that are the same as those of one or more natural amino acids. In some embodiments, the non-natural amino acids mimic one or more natural amino acids. In some embodiments, the non-natural amino acids mimic structural properties of one or more natural amino acids. In some embodiments, the non-natural amino acids mimic functional properties of one or more natural amino acids. In some embodiments, the non-natural amino acids have analogues structural properties as one or more natural amino acids. In some embodiments, the non-natural amino acids have the same functional properties as one or more natural amino acids.

In various aspects of the invention, the unnatural amino acids are used for the chemical derivatization of peptides and proteins based upon the reactivity of an aromatic amine group. In some embodiments, the non-natural amino acids are functionalized on their sidechains such that their reaction with a derivatizing molecule generates an amine linkage. In some embodiments, the non-natural amino acids are selected from amino acids having aromatic amine sidechains. In some embodiments, the non-natural amino acids comprise a masked sidechain, including a masked aromatic amine group.

In some embodiments, the non-natural amino acids comprise aromatic amine sidechains where the aromatic amine is selected from an aryl amine or a heteroaryl amine. In some embodiments, the non-natural amino acids resemble a natural amino acid in structure but contain aromatic amine groups. In some embodiments, the non-natural amino acids resemble phenylalanine or tyrosine (aromatic amino acids).

In various embodiments, the non-natural amino acids have properties that are distinct from those of one or more natural amino acids. In one embodiment, such distinct properties are the chemical reactivity of the sidechain. In one embodiment, the distinct chemical reactivity permits the sidechain of the non-natural amino acid to undergo a reaction while being a unit of a polypeptide even though the sidechains of the naturally-occurring amino acid units in the same polypeptide do not undergo the aforementioned reaction.

In one embodiment, the sidechain of the non-natural amino acid has a chemistry orthogonal to those of the naturally-occurring amino acids. In one embodiment, the sidechain of the non-natural amino acid comprises a nucleophile-containing moiety. In one embodiment, the nucleophile-containing moiety on the sidechain of the non-natural amino acid can undergo a reaction to generate an amine derivatized protein. In one embodiment, the sidechain of the non-natural amino acid comprises an electrophile-containing moiety. In one embodiment, the electrophile-containing moiety on the sidechain of the non-natural amino acid can undergo nucleophilic attack to generate an amine derivatized protein.

In one embodiment, the sidechain of the non-natural amino acid has a chemistry orthogonal to those of the naturally-occurring amino acids that allows the non-natural amino acid to react selectively with the aldehyde-substituted molecules. In one embodiment, the sidechain of the non-natural amino acid comprises an aromatic amine-containing moiety that reacts selectively with the aldehyde-containing molecule. In one embodiment, the aromatic amine-containing moiety on the sidechain of the non-natural amino acid can undergo reaction to generate an alkylated amine-derivatized protein.

In one embodiment, the sidechain of the non-natural amino acid has a chemistry orthogonal to those of the naturally-occurring amino acids that allows the non-natural amino acid to react selectively with the aldehyde-substituted linker molecules.

In one embodiment, the sidechain of the non-natural amino acid has a chemistry orthogonal to those of the naturally-occurring amino acids that allows the non-natural amino acid to react selectively with the aromatic amine-substituted molecules. In one embodiment, the sidechain of the non-natural amino acid comprises an aldehyde-containing moiety that reacts selectively with the aromatic amine-containing molecule; in one embodiment, the aldehyde-containing moiety on the sidechain of the non-natural amino acid can undergo reaction to generate an alkylated amine-derivatized protein. In one aspect related to the embodiments described in this paragraph are the modified non-natural amino acid polypeptides that result from the reaction of the derivatizing molecule with the non-natural amino acid polypeptides. Further embodiments include any further modifications of the already modified non-natural amino acid polypeptides.

### Unnatural Amino Acids and Compounds Thereof

In various aspects of the invention, the non-natural amino acid may exist as a separate molecule or may be incorporated into an amino acid sequence of any length. In various embodiments, the non-natural amino acid may exist as a separate molecule or may be incorporated into a peptide, polypeptide, or a protein of any length. In some embodiments, the amino acid sequence is a peptide or a fragment thereof, polypeptide or a fragment thereof, or protein or a fragment thereof.

In some aspects of the invention, the non-natural amino acid may exist as a separate molecule or may be incorporated into a foldamer of any length. In various embodiments, the non-natural amino acid may exist as a separate molecule or may be incorporated into a peptidomimetic foldamer, peptide, bispeptide, β-peptide, γ-peptide, δ-peptide, nucleotidomimetic foldamer, abiotic foldamer, peptoid, aedamer, aromatic oligamide foldamer, spiroligomer, or arylamine foldamer of any length. In some embodiments, the foldamer is a peptidomimetic foldamer, peptide, bispeptide, β-peptide, γ-peptide, δ-peptide, nucleotidomimetic foldamer, abiotic foldamer, peptoid, aedamer, aromatic oligamide foldamer, spiroligomer, arylamine foldamer, or chiral oligomers of pentenoic amides (COPAs).

In some embodiments, at least one of the aforementioned non-natural amino acids is incorporated into a peptide or a fragment thereof to produce non-natural amino acid peptides. In some embodiments, the peptide or a fragment thereof may further incorporate naturally-occurring or non-natural amino acids. In some embodiments, at least one of the aforementioned non-natural amino acids is incorporated into a polypeptide or a fragment thereof to produce a non-natural amino acid polypeptides. In some embodiments, the polypeptide or a fragment thereof may further incorporate naturally-occurring or non-natural amino acids. In some embodiments, at least one of the aforementioned non-natural amino acids is incorporated into a protein or a fragment thereof to produce a non-natural amino acid protein. In some embodiments, the protein or a fragment thereof may further incorporate naturally-occurring or non-natural amino acids.

In various aspects, carbonyl-substituted molecules (e.g., aldehydes, ketones, etc.) are used for the production of derivatized non-natural amino acid sequences based upon an amine linkage. In various aspect, carbonyl-substituted molecules (e.g., aldehydes, ketones, etc.) are used for the production of derivatized foldamers based upon an amine linkage. In one embodiment, aldehyde-substituted molecules are used to derivatize aromatic amine-containing non-natural amino acid sequences or foldamers via the formation of an amine linkage between the derivatizing molecule and the aromatic amine-containing non-natural amino acid sequence or foldamer. In some embodiments, the aldehyde-substituted molecules comprise a group selected from: a label; a dye; a polymer; a water-soluble polymer; a derivative of polyethylene glycol; a photocrosslinker; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide, a water-soluble dendrimer, a cyclodextrin, a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a ligand; a photoisomerizable moiety; biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; an inhibitory ribonucleic acid; a radionucleotide; a neutron-capture agent; a derivative of biotin; quantum dot(s); a nanotransmitter; a radiotransmitter, an abzyme, an activated complex activator, a virus, an adjuvant, an aglycan, an allergan, an angiostatin, an antihormone, an antioxidant, an aptamer, a guide RNA, a saponin, a shuttle vector, a macromolecule, a mimotope, a receptor, a reverse micelle, and any combination thereof. In some embodiments, the aldehyde-substituted molecules are aldehyde-substituted polyethylene glycol (PEG) molecules.

In various aspects, aromatic amine-substituted molecules (e.g., aryl amines, heteroaryl amines, etc.) are used for the production of derivatized non-natural amino acid sequences based upon an amine linkage. In various aspects, aromatic amine-substituted molecules (e.g., aryl amines, heteroaryl amines, etc.) are used for the production of foldamers based upon an amine linkage. In one embodiment, the aromatic amine-substituted molecules are used to derivatize aldehyde-containing non-natural amino acid sequences or foldamers via the formation of an amine linkage between the derivatizing molecule and the aldehyde-containing non-natural amino acid sequence or foldamer. In some embodiments, the aromatic amine-substituted molecules comprise a group selected from: a label; a dye; a polymer; a water-soluble polymer; a derivative of polyethylene glycol; a photocrosslinker; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide, a water-soluble dendrimer, a cyclodextrin, a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a ligand; a photoisomerizable moiety; biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; an inhibitory ribonucleic acid; a radionucleotide; a neutron-capture agent; a derivative of biotin; quantum dot(s); a nanotransmitter; a radiotransmitter; an abzyme, an activated complex activator, a virus, an adjuvant, an aglycan, an allergan, an angiostatin, an antihormone, an antioxidant, an aptamer, a guide RNA, a saponin, a shuttle vector, a macromolecule, a mimotope, a receptor, a reverse micelle, and any combination thereof. In some embodiments, the aromatic amine-substituted molecules are aromatic amine-substituted polyethylene glycol (PEG) molecules.

In various aspects, mono-, bi- and multi-functional linkers are used for the generation of derivatized non-natural amino acid sequences based upon an amine linkage. In various aspects, mono-, bi- and multi-functional linkers are used for the generation of foldamers based upon an amine linkage. In some embodiments, the molecular linkers (bi- and multi-functional) can be used to connect aromatic amine-containing non-natural amino acid sequences or foldamers to other molecules. In some embodiments, the aromatic amine-containing non-natural amino acid sequences or foldamers comprise an aryl amine or a heteroaryl amine sidechain.

In one embodiment, the molecular linker contains an aldehyde group at one of its termini. In some embodiments, the aldehyde-substituted linker molecules are aldehyde-substituted polyethylene glycol (PEG) linker molecules. In some embodiments, the aldehyde-containing molecular linkers comprise the same or equivalent groups on all termini so that upon reaction with an aromatic amine-containing non-natural amino acid sequence or foldamer, the resulting product is the homo-multimerization of the aromatic amine-containing non-natural amino acid sequence or foldamer. In one embodiment, the homo-multimerization is a homo-dimerization.

In some embodiments, the molecular linkers comprise at least one aldehyde group and a different group on all termini so that upon reaction with an aromatic amine-containing non-natural amino acid sequence or foldamer, the resulting product is the hetero-multimerization of the aromatic amine-containing non-natural amino acid sequence or foldamer. In some embodiments, the hetero-multimerization is a hetero-dimerization.

In some embodiments, the non-natural amino acid sequence or foldamer is linked to a water soluble polymer. In one embodiment, the water soluble polymer comprises a polyethylene glycol moiety. In one embodiment, the poly(ethylene glycol) molecule is a bifunctional polymer. In one embodiment, the bifunctional polymer is linked to a second amino acid sequence or foldamer. In one embodiment, the second amino acid sequence or foldamer is identical to the first amino acid sequence or foldamer. In one embodiment, the second amino acid sequence or foldamer is a different amino acid sequence or foldamer. In some embodiments, the non-natural amino acid amino acid sequence or foldamer comprises at least two amino acids linked to a water soluble polymer comprising a polyethylene glycol moiety. Examples of water soluble polymers include, but are not limited to: polyethylene glycol, polyethylene glycol propionaldehyde, mono C₁-C₁₀ alkoxy or aryloxy derivatives thereof (described in U.S. Pat. No. 5,252,714 which is incorporated by reference herein), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, serum albumin, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof.

In some embodiments, the non-natural amino acid sequences or foldamers comprise a substitution, addition or deletion that increases affinity of the non-natural amino acid sequence or foldamer for a receptor. In some embodiments, the non-natural amino acid sequence or foldamer comprises a substitution, addition, or deletion that increases the stability of the non-natural amino acid sequence or foldamer. In some embodiments, the non-natural amino acid sequence or foldamer comprises a substitution, addition, or deletion that increases the aqueous solubility of the non-natural amino acid sequence or foldamer. In some embodiments, the non-natural amino acid sequence or foldamer comprises a substitution, addition, or deletion that increases the solubility of the non-natural amino acid sequence or foldamer produced in a host cell. In some embodiments, the non-natural amino acid sequence or foldamer comprises a substitution, addition, or deletion that modulates protease resistance, serum half-life, immunogenicity, and/or expression relative to the amino-acid sequence or foldamer without the substitution, addition or deletion.

In some embodiments, the non-natural amino acid sequence or foldamer is an agonist, partial agonist, antagonist, partial antagonist, or inverse agonist. In some embodiments, the agonist, partial agonist, antagonist, partial antagonist, or inverse agonist comprises a non-natural amino acid linked to a water soluble polymer. In some embodiments, the water polymer comprises a polyethylene glycol moiety. In some embodiments, the amino acid sequence or foldamer comprising a non-natural amino acid linked to a water soluble polymer, for example, may prevent dimerization of the corresponding receptor. In some embodiments, the amino acid amino acid sequence or foldamer comprising a non-natural amino acid linked to a water soluble polymer modulates binding of the amino acid sequence or foldamer to a binding partner, ligand, or receptor. In some embodiments, the amino acid sequence or foldamer comprising a non-natural amino acid linked to a water soluble polymer modulates one or more properties or activities of the amino acid sequence or foldamer.

In some embodiments, the non-natural aromatic amine amino acid sequences or foldamers described herein have at least one of the following characteristics: (1) the amine moiety of the aromatic amine is a primary amine; (2) the amine moiety of the aromatic amine is a secondary amine; (3) the aromatic moiety of the aromatic amine is a heteroaromatic moiety; (4) the aromatic moiety of the aromatic amine is an aryl moiety; (5) the aromatic amine reacts with aldehyde functionalized groups; (6) is coupled to a water soluble polymer; (7) is PEGylated; (8) has increased therapeutic half-life relative to the corresponding polypeptide without the non-natural aromatic amine amino acid; (9) has increased serum half-life relative to the corresponding polypeptide without the non-natural aromatic amine amino acid; (10) has increased circulation time relative to the corresponding polypeptide without the non-natural aromatic amine amino acid; (11) has increased water solubility relative to the corresponding polypeptide without the non-natural aromatic amine amino acid; (12) has enhanced bioavailability relative to the corresponding polypeptide without the non-natural aromatic amine amino acid; (13) has modulated immunogenicity relative to the corresponding polypeptide without the non-natural aromatic amine amino acid; (14) has modulated biological activity relative to the corresponding polypeptide without the non-natural aromatic amine amino acid; (15) is part of a pharmaceutical composition; (16) is obtained from cell culture; (17) is chemically synthesized; (18) is used in library screening methods; (19) is used with arrays; (20) is used with protein arrays; (21) is used for gene expression analysis; (22) is coupled to at least one agent; (23) is coupled to a label; (24) is coupled to a dye; (25) is coupled to a polymer; (26) is coupled to a cytotoxic compound; (27) is coupled to a drug; (28) is coupled to a second protein or polypeptide or polypeptide analog; (29) is coupled to an antibody or antibody fragment; (30) is coupled to a carbohydrate; (31) is coupled to a polynucleotide; (32) is coupled to an antisense polynucleotide; (33) is coupled to a saccharide, (34) is coupled to a fluorophore, (35) is coupled to a chemically cleavable group; (36) is coupled to a photocleavable group; (37) is coupled to an energy transfer agent; (38) is coupled to a radionucleotide; (39) may be post-translationally modified; (40) may be post-translationally modified by reductive alkylation; (41) may be post-translationally modified by reductive alkylation in a pH range between about 4 to about 10; (42) may be site-specifically derivatized by post-translational reductive alkylation; (43) may be rapidly post-translationally modified by reductive alkylation at room temperature, (44) may be post-translationally modified by reductive alkylation in aqueous conditions; (45) may be post-translationally modified by reductive alkylation with stoichiometric reaction conditions; (46) may be post-translationally modified by reductive alkylation with near-stoichiometric reaction conditions; (47) may be post-translationally modified by reductive alkylation with stoichiometric-like reaction conditions; (48) is used to treat a mammal suffering from a disease, disorder or condition; (49) is used to treat a human suffering from a disease, disorder or condition; (50) is used to diagnose a mammal suffering from a disease, disorder or condition; (51) is used to diagnose a human suffering from a disease, disorder or condition; (52) is part of a sustained-release compositions; (53) the amine moiety is formed by post-translational reduction of a masked amine moiety; (54) the amine moiety is formed by post-translational reduction of an imine moiety; (55) the amine moiety is formed by post-translational reduction of an azide moiety; (56) the amine moiety is formed by post-translational reduction of a hydrazine moiety; (57) the amine moiety is formed by post-translational reduction of a nitro moiety, (58) is coupled to a pro-drug; (59) is obtained from cell lysate; (60) is ribosomally translated; (61) may be post-translationally modified by reductive alkylation in a pH range between about 4 to about 7; (62) may be post-translationally modified by reductive alkylation in a pH range between about 4 to about 5; (63) may be post-translationally modified by reductive alkylation at a pH of about 5; (64) may be post-translationally modified by reductive alkylation at a pH of about 4; (65) reacts rapidly in reductive alkylation reactions; (66) reacts in less than about 10 hours in reductive alkylation reactions; (67) reacts in less than about 8 hours in reductive alkylation reactions; (68) reacts in less than about 6 hours in reductive alkylation reactions; (68) reacts in less than about 4 hours in reductive alkylation reactions; (69) reacts in less than about 2 hours in reductive alkylation reactions; (70) reacts in less than about 1 hour in reductive alkylation reactions, or (71) reacts in less than about 30 minutes in reductive alkylation reactions.

In some embodiments, the non-natural aromatic amine amino acid sequences or foldamers have at least two of the aforementioned characteristics. In some embodiments, the non-natural aromatic amine amino acid sequences or foldamers have at least three of the aforementioned characteristics. In some embodiments, the non-natural aromatic amine amino acid sequences or foldamers have at least four of the aforementioned characteristics. In some embodiments, the non-natural aromatic amine amino acid sequences or foldamers have at least five of the aforementioned characteristics.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The invention should also be construed to include any form of a peptide having substantial homology to the peptides disclosed herein. Preferably, a peptide which is “substantially homologous” is about 60% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to amino acid sequence of the peptides disclosed herein.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The polypeptides of the invention can be post-translationally modified (i.e., modified after synthesis). For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases presenting the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The term “functionally equivalent” as used herein refers to a polypeptide according to the invention that preferably retains at least one biological function or activity of the specific amino acid sequence of wild-type BEST1.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of the wild-type BEST1 comprising peptide.

A peptide or protein of the invention may be phosphorylated using conventional methods such as the method described in Reedijk et al. (The EMBO Journal 11(4):1365, 1992).

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

(a) Tags

In a particular embodiment of the invention, the polypeptide of the invention further comprises the amino acid sequence of a tag. The tag includes but is not limited to: polyhistidine tags (His-tags) (for example H6 and H10, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

(b) Leader and Secretory Sequences

Accordingly, the polypeptides of the invention can be fused to another polypeptide or tag, such as a leader or secretory sequence or a sequence which is employed for purification or for detection. In a particular embodiment, the polypeptide of the invention comprises the glutathione-S-transferase protein tag which provides the basis for rapid high-affinity purification of the polypeptide of the invention. Indeed, this GST-fusion protein can then be purified from cells via its high affinity for glutathione. Agarose beads can be coupled to glutathione, and such glutathione-agarose beads bind GST-proteins. Thus, in a particular embodiment of the invention, the polypeptide of the invention is bound to a solid support. In a preferred embodiment, if the polypeptide of the invention comprises a GST moiety, the polypeptide is coupled to a glutathione-modified support. In a particular case, the glutathione modified support is a glutathione-agarose bead. Additionally, a sequence encoding a protease cleavage site can be included between the affinity tag and the polypeptide sequence, thus permitting the removal of the binding tag after incubation with this specific enzyme and thus facilitating the purification of the corresponding protein of interest.

(c) Targeting Sequences

The invention also relates to peptides comprising wild-type BEST1 fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

A target protein is a protein that is selected for degradation and for example may be a protein that is mutated or over expressed in a disease or condition. In another embodiment of the invention, a target protein is a protein that is abnormally degraded and for example may be a protein that is mutated or underexpressed in a disease or condition. The targeting domain can be a membrane spanning domain, a membrane binding domain, or a sequence directing the protein to associate with for example vesicles or with the nucleus. The targeting domain can target a peptide to a particular cell type or tissue. For example, the targeting domain can be a cell surface ligand or an antibody against cell surface antigens of a target tissue (e.g. retina tissue). A targeting domain may target the peptide of the invention to a cellular component.

(d) Intracellular Targeting

Combined with certain formulations, such peptides can be effective intracellular agents. However, in order to increase the efficacy of such peptides, the peptide of the invention can be provided a fusion peptide along with a second peptide which promotes “transcytosis”, e.g., uptake of the peptide by epithelial cells. To illustrate, the peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the RLP can be provided a fusion polypeptide with all or a portion of the antenopedia III protein.

To further illustrate, the peptide of the invention can be provided as a chimeric peptide which includes a heterologous peptide sequence (“internalizing peptide”) which drives the translocation of an extracellular form of the peptide across a cell membrane in order to facilitate intracellular localization of the peptide. In this regard, the peptide is one which is active intracellularly. The internalizing peptide, by itself, is capable of crossing a cellular membrane by, e.g., transcytosis, at a relatively high rate. The internalizing peptide is conjugated, e.g., as a fusion protein, to a peptide comprising wild-type BEST1. The resulting chimeric peptide is transported into cells at a higher rate relative to the peptide alone to thereby provide a means for enhancing its introduction into cells to which it is applied.

(e) Peptide Mimetics

In other embodiments, the subject compositions are peptidomimetics of the peptide of the invention. Peptidomimetics are compounds based on, or derived from, peptides and proteins. The peptidomimetics of the present invention typically can be obtained by structural modification of a known sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimotopes of the subject peptides can be provided. Such peptidomimetics can have such attributes as being non-hydrolysable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of side chain replacements which can be carried out to generate the peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

Moreover, other examples of mimotopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimotope can be obtained by, for example, screening libraries of natural and synthetic compounds for compounds capable of binding to the peptide of the invention. A mimotope can also be obtained, for example, from libraries of natural and synthetic compounds, in particular, chemical or combinatorial libraries (i.e., libraries of compounds that differ in sequence or size but that have the same building blocks). A mimotope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimotopes by, for example, computer modelling, the predicted mimotope structures can then be produced by, for example, chemical synthesis, recombinant DNA technology, or by isolating a mimotope from a natural source (e.g., plants, animals, bacteria and fungi).

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editorsE. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.) By way of example, a RLP or chimeric protein may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the wild-type BEST1 comprising peptide or chimeric protein fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (See Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Macrocycles

In various aspects, the present invention also relates, in part, to macrocycles comprising one or more unnatural amino acids described herein. For example, in various aspects, the macrocycle is a compound or salt thereof having the structure of Formula (IX)

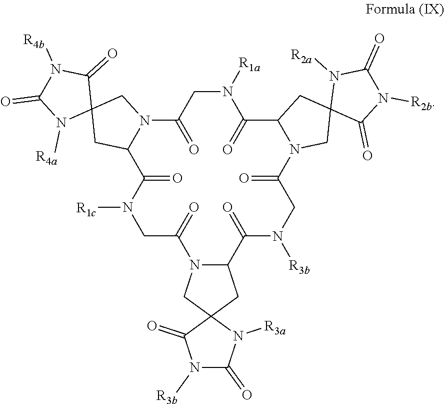

In some embodiments, R₁ₐ, R_{1b}, R_{1c}, R₂ₐ, R_{2b}, R₃ₐ, R_{3b}, R₄ₐ, or R_{4b} is H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted-Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural amino acid, unnatural amino acid, or

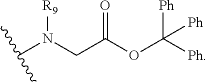

In various embodiments, Y is C, N, O, S, or P.

In some embodiments, o is an integer represented by 0, 1, 2, or 3. In some embodiments, p is an integer represented by 0, 1, 2, or 3.

In some embodiments, R₅, R₆, or R₇ is H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, or sulfoxy.

In some embodiments, the macrocycle is a compound having the structure of

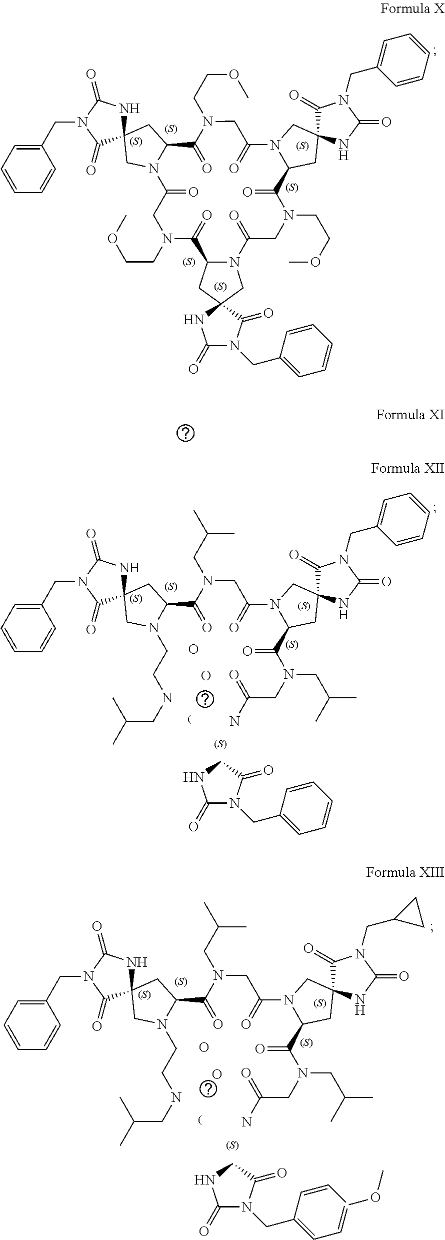

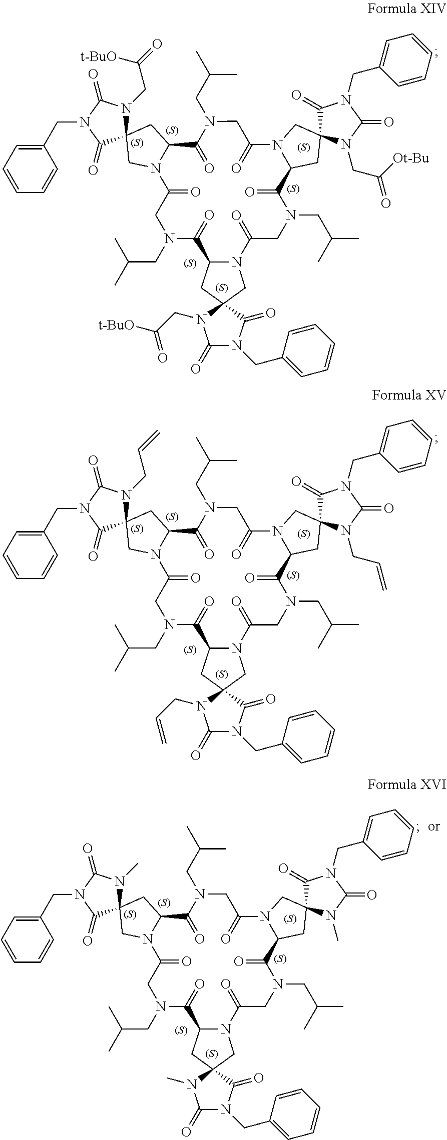

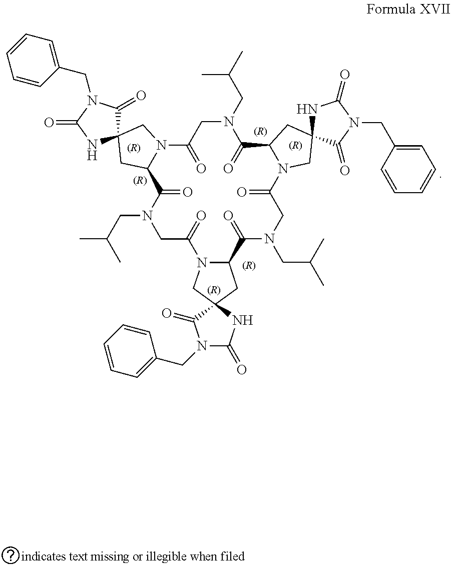

In some embodiments, the macrocycle comprises at least one metal ion. In one embodiment, the metal ion is a metal cation. Example of metal ions include, but are not limited to Li^{tm}, Na^{tm}, or K^{tm}.

### Compositions

In various aspects, the present invention also provides a composition comprising one or more unnatural amino acids, one or more unnatural amino acid sequences, one or more foldamer, one or more macrocycles described herein, or any combination thereof.

In some embodiments, the composition comprises N-oxides, crystalline forms, or pharmaceutically acceptable salts of the non-natural amino acids, non-natural amino acid sequences, foldamers, or macrocycles described herein. In certain embodiments, non-natural amino acids, non-natural amino acid sequences, foldamers, or macrocycles may exist as tautomers. All tautomers are included within the scope of the non-natural amino acids, non-natural amino acid sequences, foldamers, or macrocycles presented herein.

In some embodiments, the composition comprises non-natural amino acids, non-natural amino acid sequences, foldamers, or macrocycles described herein that can exist in unsolvated as well as solvated forms with pharmaceutically acceptable solvents such as water, ethanol, and the like. The solvated forms of the non-natural amino acids, non-natural amino acid polypeptides and modified non-natural amino acid polypeptides presented herein are also considered to be disclosed herein.

In some embodiments, the composition comprises non-natural amino acids, non-natural amino acid sequences, foldamers, or macrocycles and reagents for producing either of the aforementioned compounds that are acidic and may form a salt with a pharmaceutically acceptable cation. In some embodiments, the composition comprises non-natural amino acids, non-natural amino acid sequences, foldamers, or macrocycles and reagents for producing the aforementioned compounds that can be basic and accordingly, may form a salt with a pharmaceutically acceptable anion. All such salts, including di-salts are within the scope of the compositions described herein and they can be prepared by conventional methods. For example, salts can be prepared by contacting the acidic and basic entities, in either an aqueous, non-aqueous or partially aqueous medium. The salts are recovered by using at least one of the following techniques: filtration, precipitation with a non-solvent followed by filtration, evaporation of the solvent, or, in the case of aqueous solutions, lyophilization.

In one embodiment, the composition is a pro-drug. Thus, in some embodiments, the composition is metabolized upon administration to a subject in need thereof to produce a metabolite that is then used to produce a desired effect, including a desired therapeutic effect.

### Methods

The present invention also relates to methods, compositions, techniques, and strategies for making, purifying, characterizing, and using non-natural amino acids, non-natural amino acid sequences, foldamers, macrocycles, or compositions described herein.

In one aspect are methods, compositions, techniques, and strategies for derivatizing a non-natural amino acid and/or a non-natural amino acid sequence and/or foldamer. In one embodiment, such methods, compositions, techniques, and strategies involve chemical derivatization, biological derivatization, physical derivatization, or any combination thereof. In some embodiments, the derivatizations are regioselective. In some embodiments, the derivatizations are regiospecific.

In some embodiments, the derivatizations are stoichiometric, near stoichiometric, or stoichiometric-like in both the non-natural amino acid containing reagent and the derivatizing reagent. In some embodiments, the methods comprise a stoichiometric, near stoichiometric, or stoichiometric-like incorporation of a desired group onto a non-natural amino acid sequence or foldamer. In some embodiments, the provided strategies, reaction mixtures, or synthetic conditions comprise stoichiometric, near stoichiometric, or stoichiometric-like incorporation of a desired group onto a non-natural amino acid sequence or foldamer.

In some embodiments, the derivatizations are rapid at ambient temperature. In some embodiments, the derivatizations occur in aqueous solutions. In some embodiments, the derivatizations occur at a pH between about 4 and about 10. In some embodiments, the derivatizations occur at a pH between about 4 and about 7. In some embodiments, the derivatizations occur at a pH between about 4 and about 5. In some embodiments, the derivatizations occur at a pH of about 5. In some embodiments, the derivatizations occur at a pH of about 4.

In various aspects, the methods for derivatizing a non-natural amino acid or a non-natural amino acid sequence or foldamer comprise a reaction of aromatic amines and aldehyde reactants to form alkylated amine-derivatized non-natural amino acid or a non-natural amino acid sequence or foldamer. In some embodiments, the method comprises a reductive alkylation of aromatic amine- and aldehyde-containing reactants to generate alkylated amine-derivatized non-natural amino acid or non-natural amino acid sequence or foldamer adduct. In some embodiments, the method comprises derivatization of aromatic amine-containing non-natural amino acid, non-natural amino acid sequence, or foldamer with aldehyde-functionalized polyethylene glycol (PEG) molecules.

In some embodiments, the method comprises the chemical synthesis of aldehyde-substituted molecules for the derivatization of aromatic amine-substituted proteins. In one embodiment, the method comprises a preparation of aldehyde-substituted molecules suitable for the derivatization of aromatic amine-containing non-natural amino acid sequences or foldamers. In one embodiment, the method for the preparation of aldehyde-substituted molecules provides access to a wide variety of site-specifically derivatized polypeptides. In one embodiment, the method comprises synthesizing aldehyde-functionalized polyethylene glycol (PEG) molecules. In some embodiments, the aldehyde-substituted molecules allow for the site-specific derivatization of aromatic amine-containing non-natural amino acids via reductive alkylation of the aromatic amine moiety to form an alkylated amine-derivatized polypeptide in a site-specific fashion. In various embodiments, the aldehyde-substituted molecule can include proteins, peptides, other polymers (non-branched and branched) and/or small molecules.

In some embodiments, the method comprises a chemical derivatization of aromatic amine-substituted non-natural amino acid sequences or foldamers using an aldehyde-containing bi-functional linker. In one embodiment, the method comprises attaching an aldehyde-substituted linker to an aromatic amine-substituted protein via a reductive alkylation reaction to generate an amine linkage. In some embodiments, the aromatic amine-substituted non-natural amino acid is an aryl amine or a heteroaryl amine-substituted non-natural amino acid. In some embodiments, the non-natural amino acid sequences or foldamers are derivatized site-specifically and/or with precise control of three-dimensional structure, using an aldehyde-containing bi-functional linker. In one embodiment, such methods are used to attach molecular linkers (mono- bi- and multi-functional) to aromatic amine-containing non-natural amino acid sequences or foldamers, wherein at least one of the linker termini contains an aldehyde group which can link to the aromatic amine-containing non-natural amino acid sequence or foldamers via an amine linkage. In various embodiments, the linkers are used to connect the aromatic amine-containing non-natural amino acid sequences or foldamers to other molecules, including by way of example, proteins, other polymers (branched and non-branched) and small molecules.

In various aspects, the invention also provides a method for mimicking one or more natural amino acid in a subject in need thereof. In various embodiments, the method of mimicking one or more natural amino acid comprises one or more unnatural amino acids, amino acid sequences, foldamers, macrocycles, and/or compositions described herein.

In one embodiment, the method of mimicking one or more natural amino acid comprises administering to the subject a therapeutically effective amount of one or more unnatural amino acids of the present invention. In one embodiment, the method of mimicking one or more natural amino acid comprises administering to the subject a therapeutically effective amount of one or more unnatural amino acid sequences of the present invention. In one embodiment, the method of mimicking one or more natural amino acid comprises administering to the subject a therapeutically effective amount of one or more foldamers of the present invention. In one embodiment, the method of mimicking one or more natural amino acid comprises administering to the subject a therapeutically effective amount of one or more macrocycles of the present invention. In one embodiment, the method of mimicking one or more natural amino acid comprises administering to the subject a therapeutically effective amount of one or more compositions of the present invention. In some embodiments, the method of mimicking one or more natural amino acid comprises administering to the subject a therapeutically effective amount of a combination of one or more unnatural amino acids, unnatural amino acid sequences, foldamers, macrocycles, and compositions of the present invention.

In various embodiments, the unnatural amino acids, amino acid sequences, foldamers, macrocycles, and/or compositions thereof mimic the function of the natural amino acid. In various embodiments, the unnatural amino acids, amino acid sequences, foldamers, macrocycles, and/or compositions thereof mimic the structure of the natural amino acid.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Kits

The present invention also pertains to kits useful in the methods of the invention. Such kits comprise various combinations of components useful in any of the methods described elsewhere herein, including for example, at least one unnatural amino acid, unnatural amino acid sequence, foldamer, macrocycle, and/or formulation thereof for mimic one or more natural amino acid, and instructional material. In some embodiments, such kits comprise various combinations of components useful in any of the methods described elsewhere herein, including for example, at least one unnatural amino acid sequence or a formulation thereof for mimicking one or more natural amino acid sequence, and instructional material. For example, in one embodiment, the kit comprises components useful for mimicking one or more natural amino acid in a subject in need thereof. In one embodiment, the kit comprises components useful for mimicking one or more natural amino acid sequence in a subject in need thereof.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Bifunctional Prolines Incorporated into Peptidomimetics, Macrocycles, and Metal-Binding Q-Proline Macrocycles (QPM)

The present example discloses a synthesis of a new, conformationally-constrained quaternary residue, Q-proline (Q-Pro), that is also derived from L-trans-4-hydroxyproline, utilizing chemistry described in a previous work (Northrup J D et al., 2017, J. Org. Chem., 82:3223). The synthesis of Q-proline is straightforward even on multigram scale with minimal purification required. Q-pro residues have two stereocenters that are controlled in the synthesis as well as two functional groups that are introduced after the stereochemistry is set. This allows a synthesis of large quantities of a stereochemically-pure, unfunctionalized building-block, and later attach functional groups to create functionalized Q-Pro residues. This decoupling of functional group installation from setting of stereochemistry allows the stereochemistry of the building block to be introduced, the diastereomers separated, and then functional groups are added, avoiding mixtures of stereoisomers that are difficult to separate. Given their pre-organized core, and the enormous diversity afforded by incorporating two functional groups through simple alkylation reactions, these Q-Pro residues could prove useful in the development of peptidomimetics with new properties.

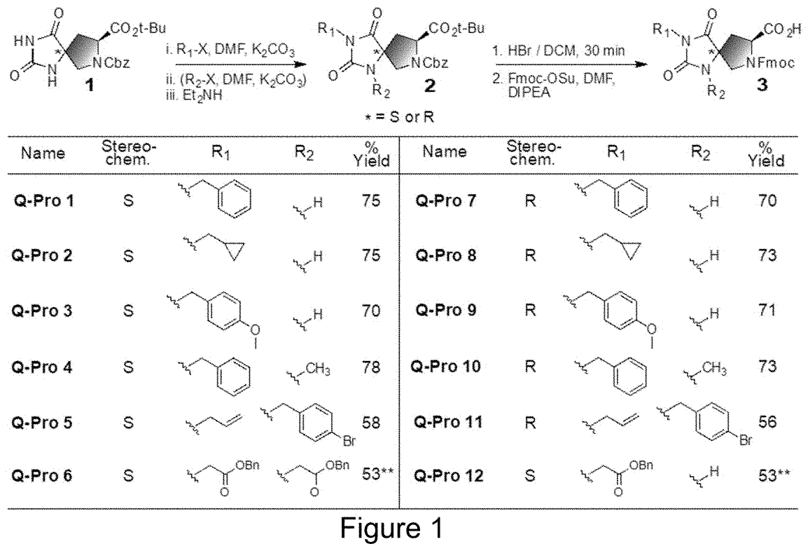

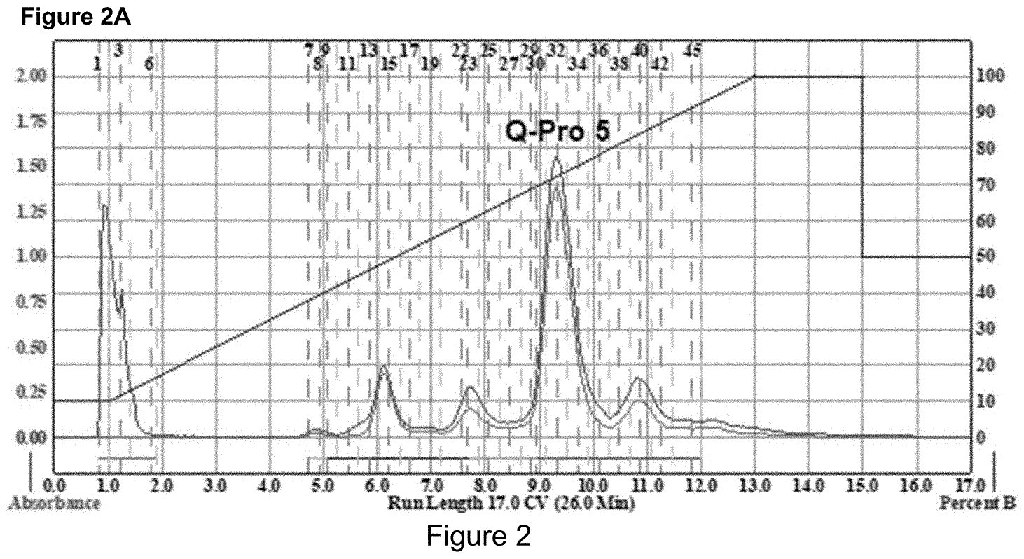

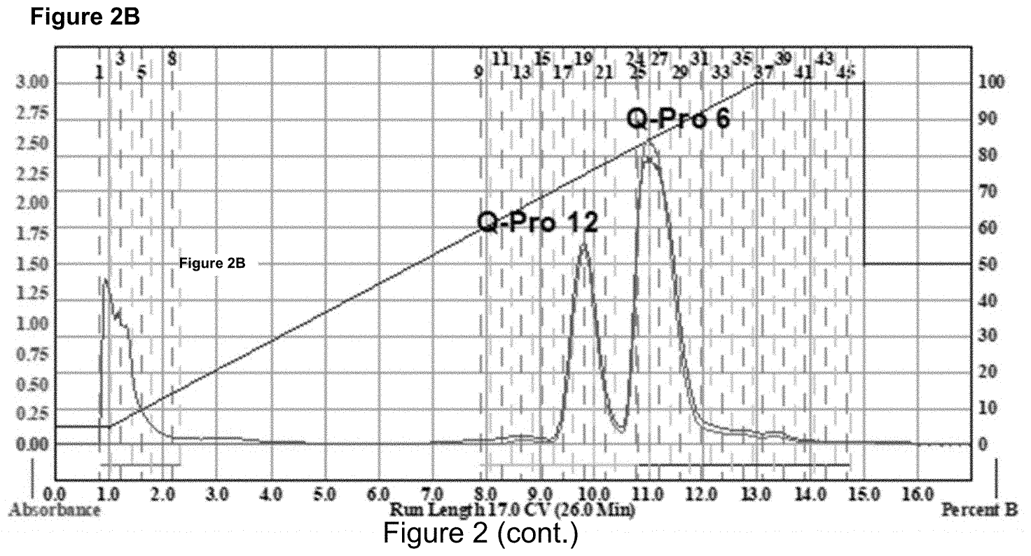

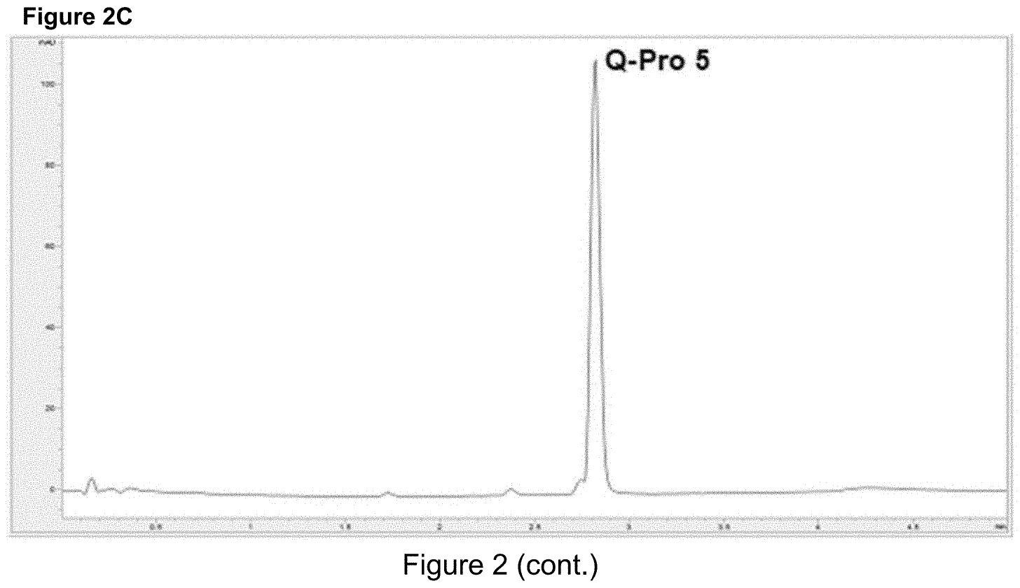

Starting from the stereochemically pure hydantoin (compound 1) and utilizing the recently published alkylation strategy (Northrup J D et al., 2017, J. Org. Chem., 82:3223), mono- and difunctionalized proline amino acids were synthesized on a 10 gram scale of compound 1 as shown in FIG. 1. A facile protecting group interchange from N-benzyloxycarbonyl (Cbz) to N-9-fluorenylmethyloxycarbonyl (Fmoc) afforded the compounds 4-15 (also referred to as QPro 1-12), which were purified by reversed-phase flash chromatography (see FIG. 2). This synthesis over 3 steps yields the Q-prolines 4-15 in moderate to good overall yields (55-78%) from compound 1.

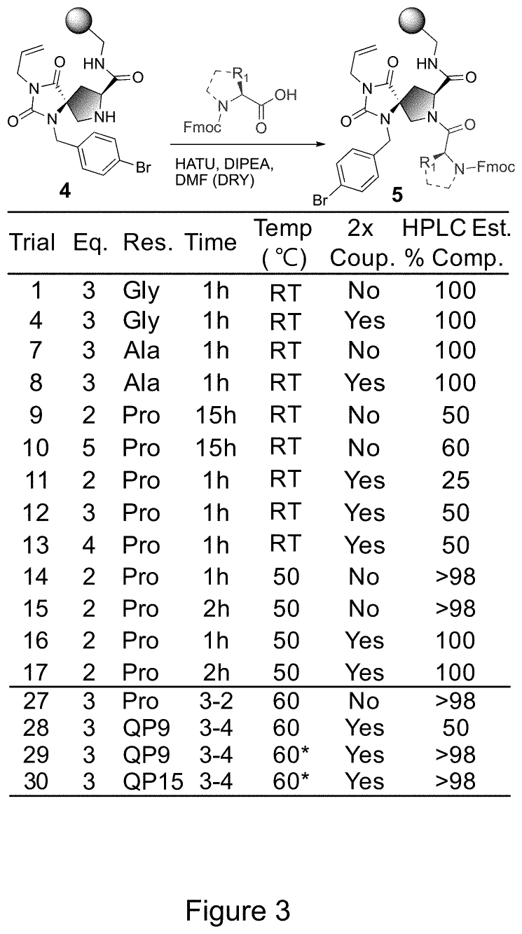

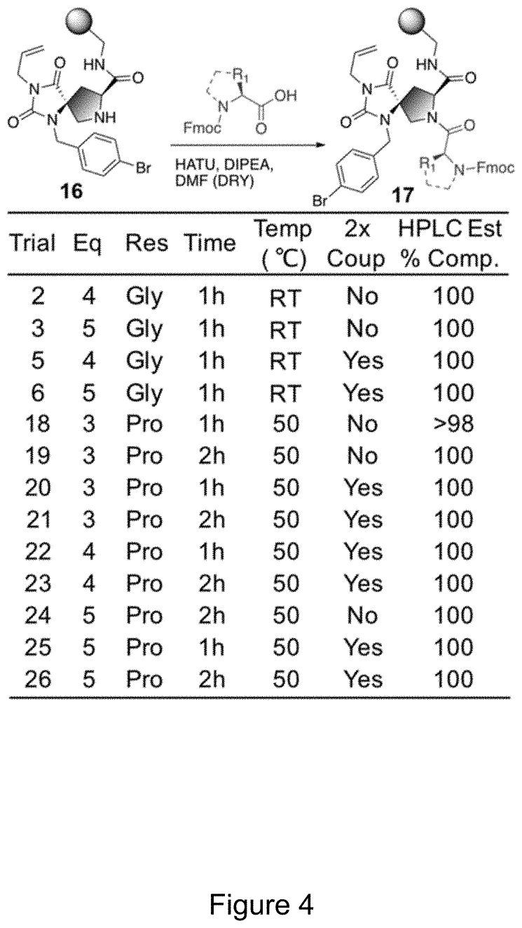

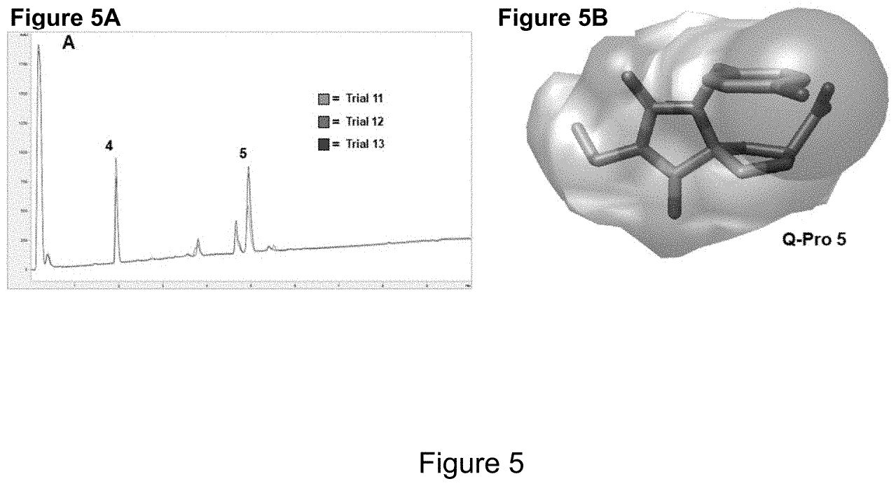

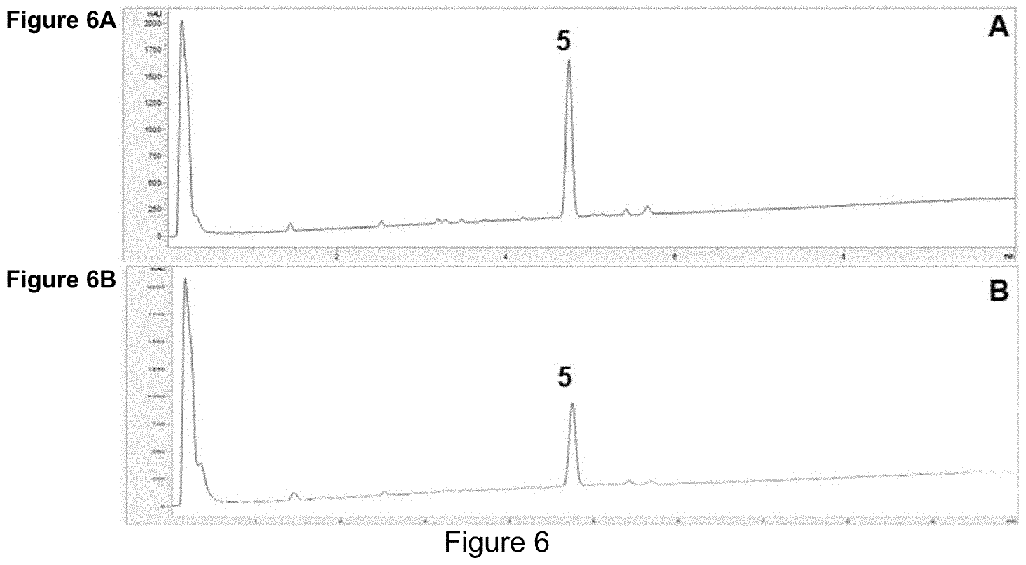

Next, the Q-proline derivatives were evaluated for standard Fmoc-SPPS. Q-Pro 5 was loaded onto rink amide resin to provide compound 4, followed by quantitative Fmoc release. A proteogenic amino acid (glycine, alanine, or proline) was then coupled to the resin bound Q-Pro (FIG. 3) to synthesize dipeptide 5. For proline, regardless of equivalents or double couplings (FIG. 3, Trials 9-13), quantitative coupling to the resin bound Q-proline could not be achieved. More trials and explanation of estimated percent completion are shown in FIG. 4. Trials 11-13 are shown in FIG. 5A, where a majority of the starting material, compound 4, remains. Although not bound by any particular theory, it was hypothesizes that this is due to steric clashes associated with the functional group on the amide of the hydantoin (FIG. 5B). In previous studies (Zhao Q et al., 2012, J. Org. Chem., 77:4784.), a group on this amide displayed a steric blocking effect. Carrying out the coupling reactions at higher temperatures allows for quantitative couplings with proline (FIG. 3, Trials 14-17). To facilitate more rapid syntheses of peptides incorporating hindered Q-Pro residues, a microwave reactor was utilized (FIG. 3, Trials 27-30) based on protocols set forth in Murray and Gellman (Murray J K et al., 2005, Org. Lett., 7:1517.). Proteogenic proline was incorporated using a 3 min ramp to a hold temperature of 60° C., followed by a 2 min hold, which provided near quantitative coupling (yields >98%.) As shown in Trial 28, coupling of two Q-Pro was also attempted; however, with the same conditions utilized for proline, only approximately 50% coupling was achieved. By employing compressed air cooling during the hold time (Trials 29-30)—which forces continuous irradiation by the microwave to maintain temperature—two Q-Pro residues were successfully coupled with excellent yield (FIG. 6).

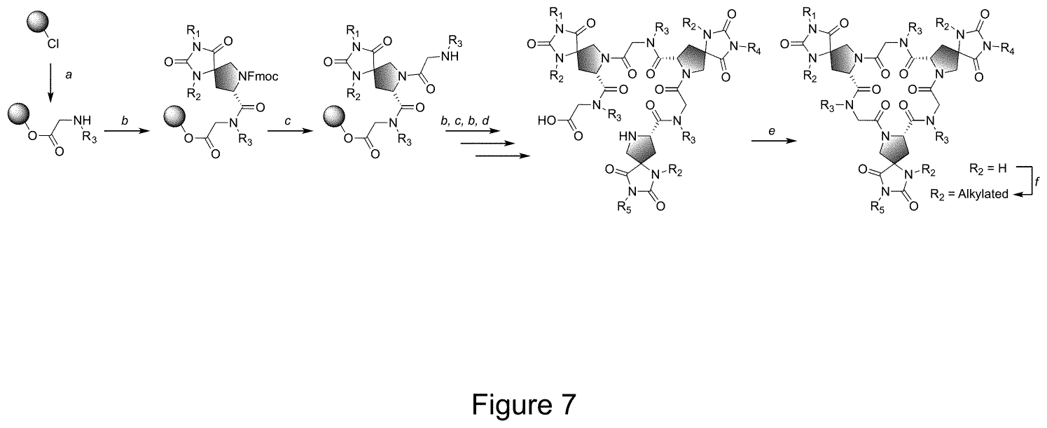

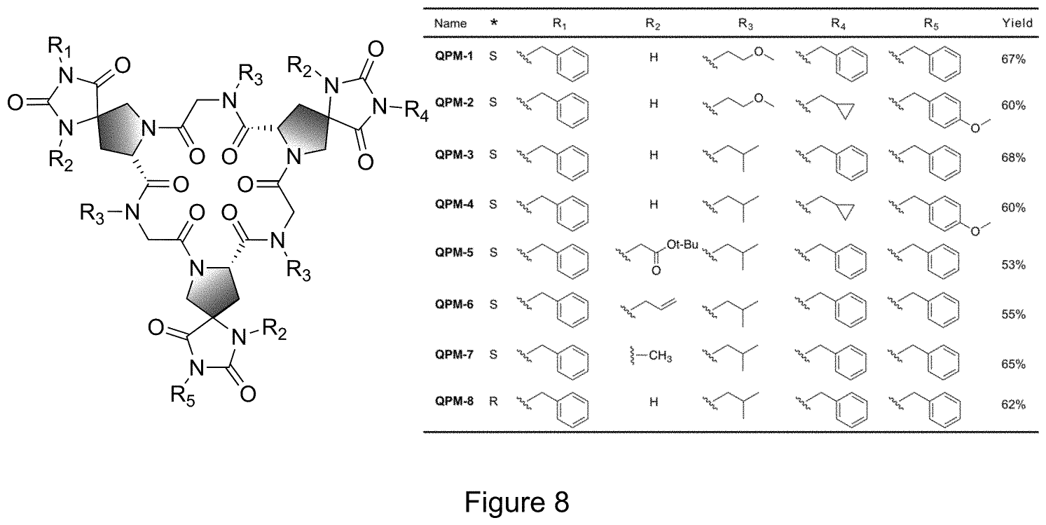

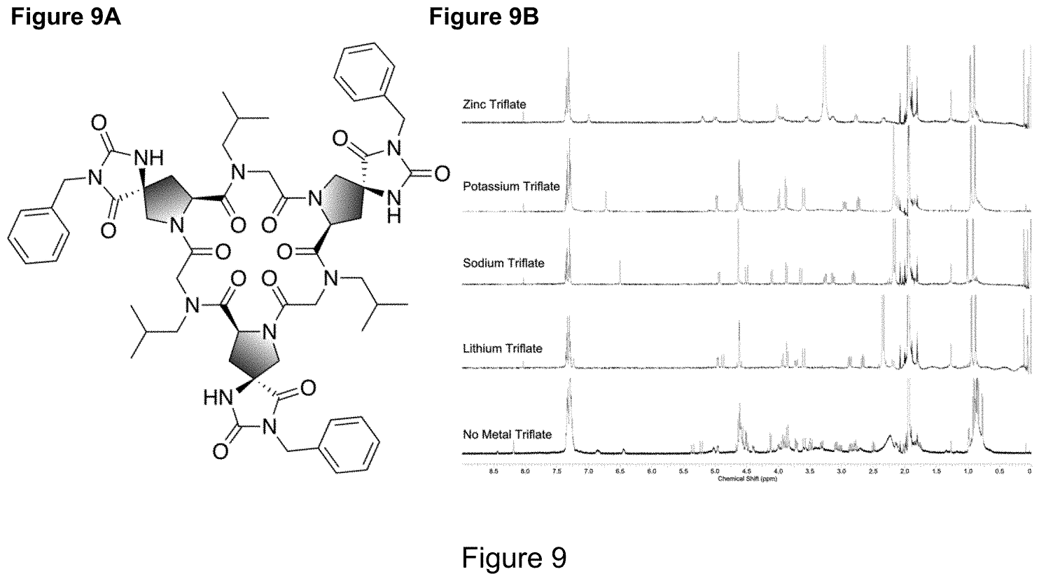

A major goal of this work was to create a scaffold that adopts a well-defined 3-dimensional structure in solution. Although not bound by any particular theory, it was predicted that by incorporating these Q-Pro residues into peptoid macrocycles, scaffolds that display functional groups above and around the periphery of the macrocyclic core could be developed. As shown in FIG. 7, the macrocycles were synthesized by loading bromoacetic acid onto 2-Cl-trityl chloride resin using chemistry developed by Shin (Shin S B et al., 2007, J. Am. Chem. Soc., 129:3218), followed by standard Fmoc-SPPS or peptoid couplings to make mono-alkylated precursors. Macrocyclization of the linear precursors was achieved in yields ranging from 60-67% utilizing either a syringe pump or extreme dilution to provide the monoalkylated Q-proline macrocycles (for exact structures see FIG. 8). The alkylation chemistry (Northrup J D et al., 2017, J. Org. Chem., 82:3223) was applied to to Q-Pro Macrocycles (QPM)-3 (FIG. 9) to modify the amide functional group (R₂), synthesizing QPM-5 and QPM-6 (FIG. 8). This late stage alkylation is a unique property of Q-Pro derivatives, allowing for the addition of reactive functional groups to a molecule as the final step in the synthesis.

When injected on an HPLC, the macrocycles elute in multiple peaks at room temperature; however, by heating the column during elution (60° C.), the peaks coalesce, sometimes into a single peak. This suggests that at room temperature, these macrocycles might exist in multiple slowly-interconverting conformations. To determine if the conformations were interconverting at room temperature—or whether they were locked into a specific conformation—the corresponding compounds represented by these HPLC peaks were isolated on an analytical column and left in solution overnight. Upon reinjection, the samples equilibrated to provide a mixture of peaks, consistent with multiple slowly interconverting conformations at room temperature.

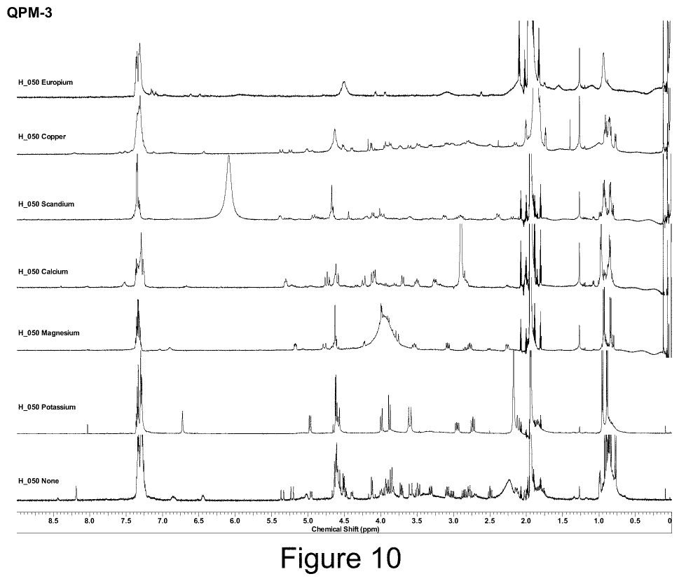

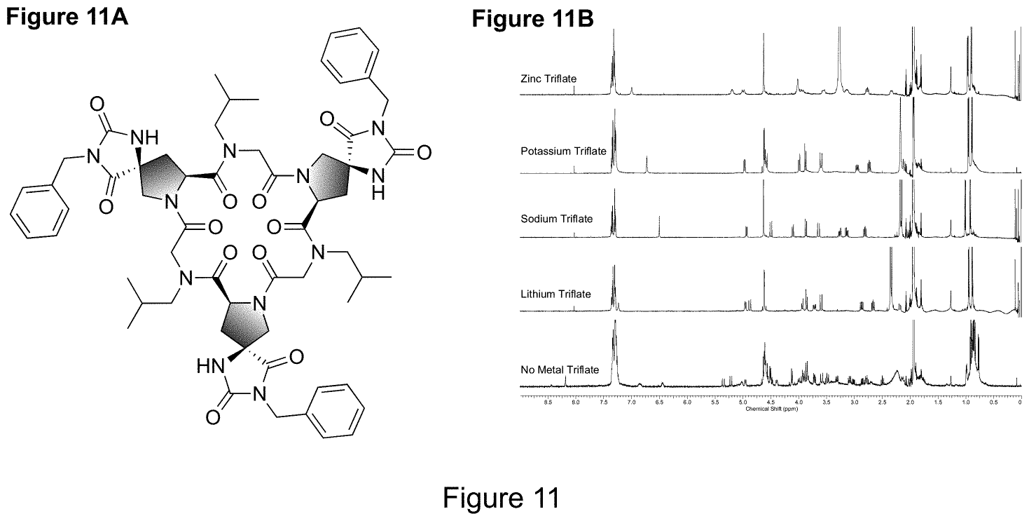

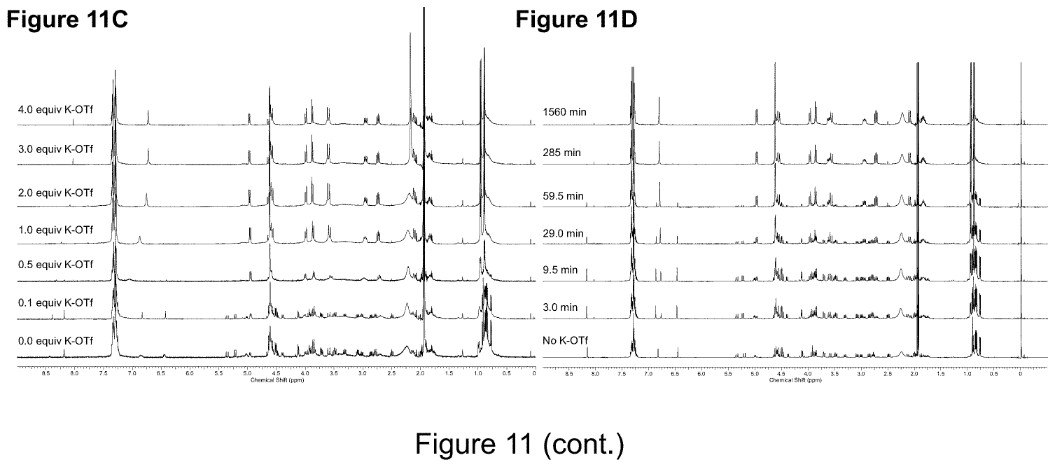

Several research groups have shown that 6-residue (18-member) macrocycles exhibit multiple slowly interconverting conformations at room temperature (Izzo I et al., 2013, Org. Lett., 15:598; Tedesco C et al., 2014, CrystEngComm, 16:3667; Meli A et al., 2016, Angew. Chem., 128:4757.); however, these conformations can be biased by internal steric interactions such as a well-placed methyl group (D'Amato A et al., 2018, Org. Lett., 20:640) or via external sources such as cation-carbonyl interactions. To test whether these QPM could adopt well-defined structures in solution, multiple metal triflates were screened against QPM-3. The alkali triflates (lithium, sodium, and potassium) showed the best results for metal binding, each giving clean NMR spectra indicative of three-fold symmetry with 4 equiv. of the metal triflate, whereas other triflates (magnesium, zinc, calcium, scandium, copper and europium, FIG. 10) did not provide symmetric structures of the macrocycles. To test the optimum conditions for obtaining single structures in solution, the equiv. of potassium triflate from 0.1 to 4.0 equiv. was varied. It was found that 2.0 equiv. of metal triflate was required to obtain a well-defined NMR spectrum (FIG. 11C); however, simplification of the spectrum was noticeable after addition of just 0.5 equiv. of potassium triflate. Furthermore, subsequent experiments demonstrated that after addition of potassium triflate, 5 hours are required for the spectra to become sharp and simple (FIG. 11D). Although not bound by any particular theory, it was hypothesized that this indicates the macrocycles adopt multiple, slowly-interconverting conformations in the absence of metal, and that on introduction of metal they become more ordered. The five-hour time for the spectra to become ordered suggests a barrier to conversion of at least 17 kcal.

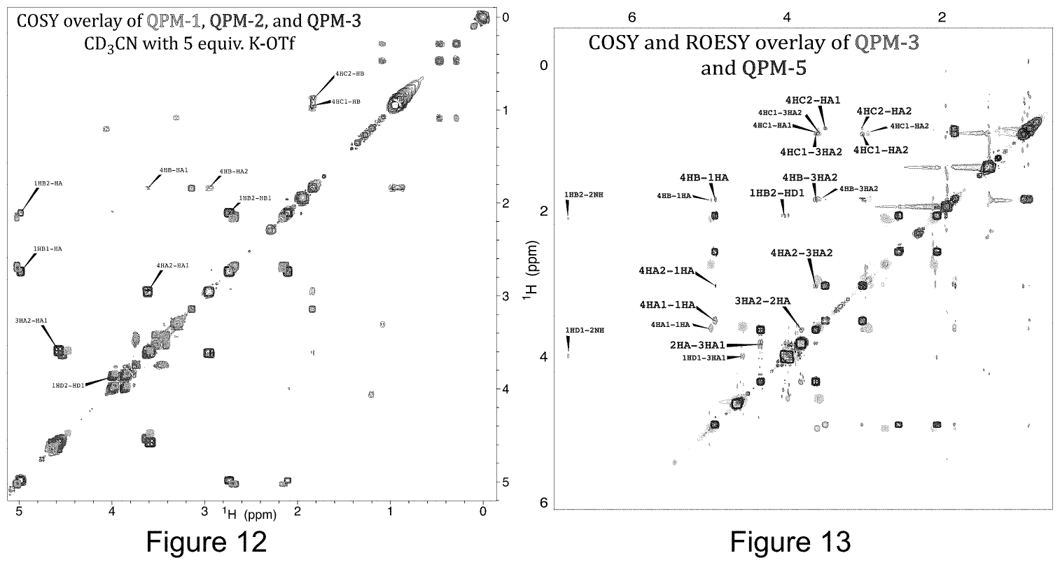

With the presumed ability to obtain single-conformation structures in solution of both symmetric and asymmetric macrocycles, it was tested if changes in the functional groups at either the R₁ or R₃ positions would impart structural shifts of the Q-proline macrocycles. Multiple 2D NMR spectra were obtained for each macrocycle (COSY, HSQC, HMBC, ROESY). An overlay of COSY spectra for QPM-1, QPM-2, and QPM-3 show remarkable similarities in shifts for the α (4.98-5.02 ppm), β (2.10-2.16 ppm and 2.70-2.74 ppm), and 6 (3.83-3.87 ppm and 3.96-4.00 ppm) protons of the proline ring (FIG. 12). Furthermore, ROESY data for each macrocycle corroborates these findings: for each macrocycle of the series, the proline a proton (group 1, HA) correlates to the “α” proton of the peptoid functional group (group 4, HA1). The amide proton of the hydantoin (R₂=H) correlates to both a β proton (group 1, HB2) and a δ proton (group 1, HD1), indicating that the proline envelope most likely favors the Cγ-endo conformation for all the macrocycles. The COSY and ROESY data suggest that despite any functional group changes at the R₁ or R₃ positions, the overall structure of the macrocyclic core remains fairly uniform in the presence of an alkali cation.

The dialkylated macrocycles QPM-5 through QPM-7 show similar conformational flexibility and alkali-metal binding as the monoalkylated macrocycles. However, a marked difference in the proline envelope conformation for the dialkylated macrocycles was observed, as shown by a ROESY overlay of QPM-3 and QPM-5 (see FIG. 13). Upon addition of any functional group to the R₂ position, the proline envelope flips into a Cγ-exo conformation, evidenced by the lack of any correlation between the new R₂ group and the β or δ protons of the proline ring; furthermore, a new correlation appears between a β proton (group 1, HB1) and a 6 proton (group 1, HD2) that was not present in the monoalkylated macrocycles. Although not bound by any particular theory, it was hypothesized that a steric clash exists between the new R₂ group and the proline carbonyl in the Cγ-endo conformation. The envelope flip suggests that rather than pointing towards the core of the macrocycle, the R₂ group is pointing away from the proline ring.

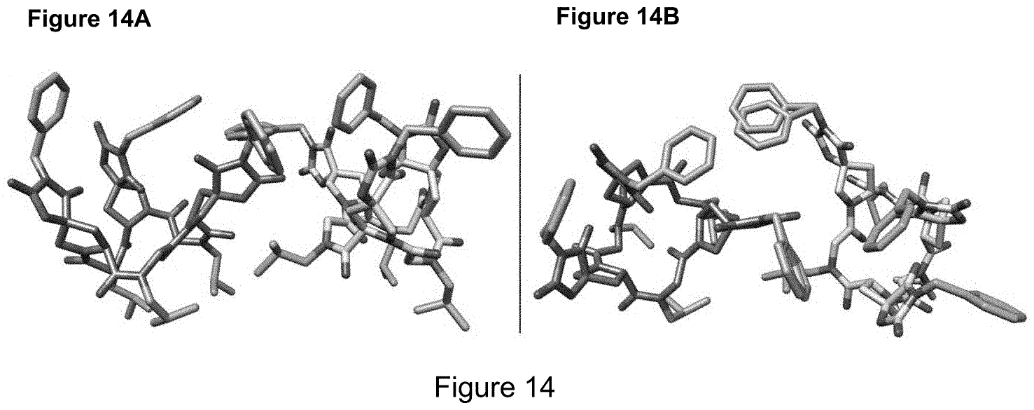

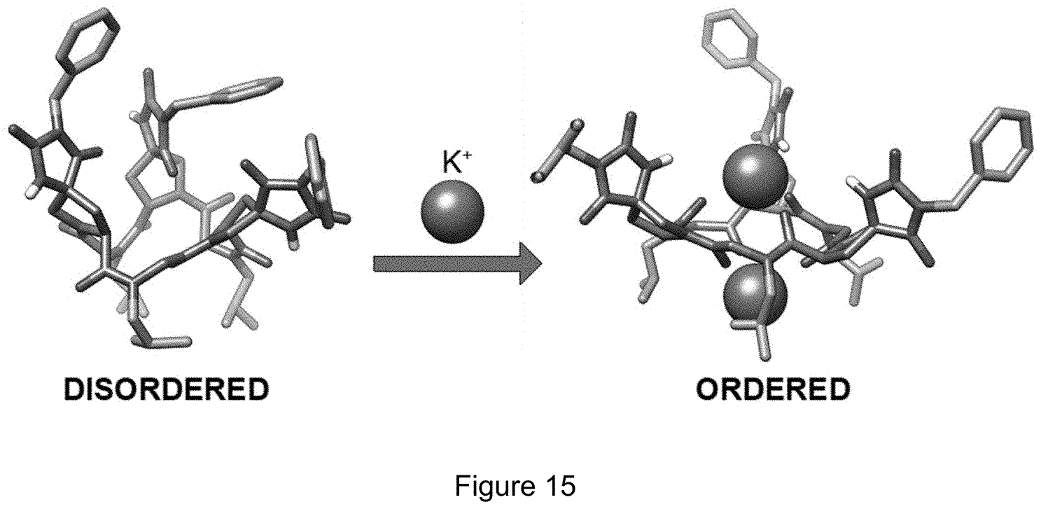

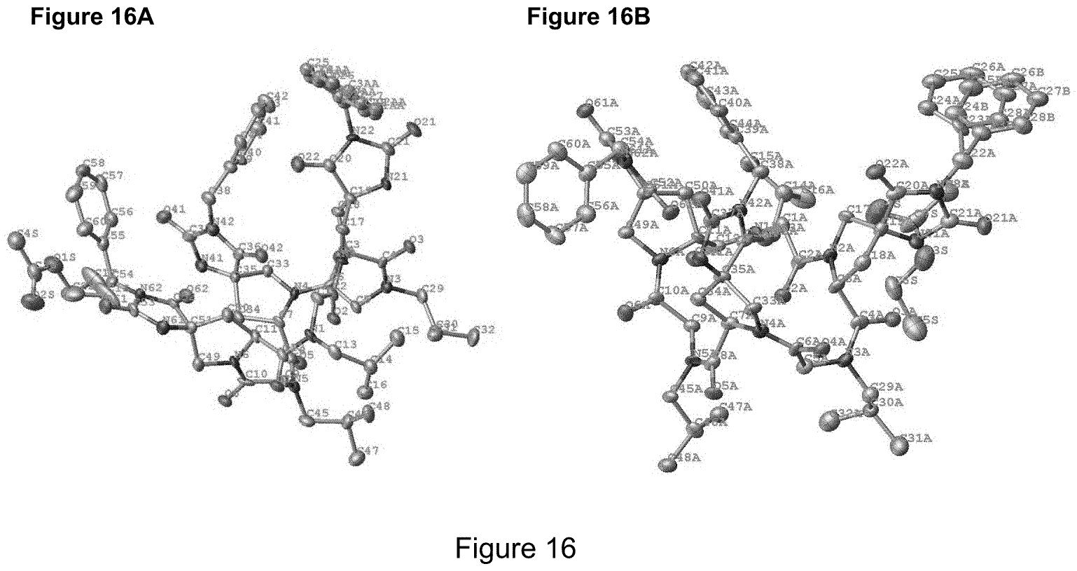

Many approaches for crystallizing these macrocycles were attempted, both in the absence and presence of the metal cations; in all instances, it was not able to obtain crystals of the macrocycles. Following work pioneered by Stephen Kent on racemic crystallization of proteins (Yeates T O et al., 2012, Annu. Rev. Biophys., 41:41), QPM-8, the enantiomer to QPM-3, was synthesized with the rationale that an achiral, racemic complex has significantly more three-dimensional space groups available to it than does a chiral molecule. By synthesizing each macrocycle stereochemically pure, and then mixing equimolar quantities into ethyl acetate with hexanes as the diffusion solvent, microcrystals of the racemate of QPM-3 were able to be obtained (FIG. 14, FIG. 16, and Table 1 through Table 3).

TABLE 1
Crystal data and structure refinement for QPM-3.

| Identification code | QPM-3 | |
|---|---|---|
| Empirical formula | C₆₄H₈₀N₁₂O₁₄ | |
| Formula weight | 1241.40 | |
| Temperature/K. | 100 | (2) |
| Crystal system | triclinic | |
| Space group | P-1 | |
| a/Å | 11.642 | (2) |
| b/Å | 18.450 | (4) |
| c/Å | 32.122 | (6) |
| α/° | 104.72 | (3) |
| β/° | 94.74 | (3) |
| γ/° | 105.21 | (3) |
| Volume/Å³ | 6356 | (3) |

| | |
|---|---|
| Z | 4 |
| ρ_{calc}g/cm³ | 1.297 |
| μ/mm⁻¹ | 0.087 |
| F(000) | 2640.0 |
| Crystal size/mm³ | 0.5 × 0.01 × 0.01 |
| Radiation | synchrotron (λ = 0.68898) |
| 2Θ range for data collection/° | 2.928 to 33.364 |
| Index ranges | −9 ≤ h ≤ 9, −15 ≤ k ≤ 15, |
| | −25 ≤ l ≤ 26 |
| Reflections collected | 12333 |
| Independent reflections | 6839 [Rᵢₙₜ = 0.0557, R_{sigma} = 0.0935] |
| Data/restraints/parameters | 6839/462/1696 |
| Goodness-of-fit on F² | 1.075 |
| Final R indexes [I >= 2σ (I)] | R₁ = 0.0874, wR₂ = 0.2278 |
| Final R indexes [all data] | R₁ = 0.1036, wR₂ = 0.2549 |
| Largest diff. peak/hole/e Å⁻³ | 0.84/−0.38 |

TABLE 2
Bond Lengths for QPM-3.

| Atom | Atom | Length/Å | Atom | Atom | Length/Å |
|---|---|---|---|---|---|
| O1A | C12A | 1.222(11) | N1 | C1 | 1.458(11) |
| O2A | C2A | 1.243(11) | N1 | C12 | 1.353(11) |
| O3A | C4A | 1.253(11) | N1 | C13 | 1.460(10) |
| O4A | C6A | 1.245(11) | N2 | C2 | 1.331(12) |
| O5A | C8A | 1.217(10) | N2 | C3 | 1.477(11) |
| O6A | C10A | 1.225(10) | N2 | C17 | 1.464(11) |
| O21A | C21A | 1.211(12) | N3 | C4 | 1.345(11) |
| O22A | C20A | 1.216(12) | N3 | C5 | 1.464(10) |
| O41A | C37A | 1.218(12) | N3 | C29 | 1.457(11) |
| O42A | C36A | 1.204(10) | N4 | C6 | 1.353(12) |
| O61A | C53A | 1.196(12) | N4 | C7 | 1.481(11) |
| O62A | C52A | 1.214(11) | N4 | C33 | 1.436(10) |
| N1A | C1A | 1.456(10) | N5 | C8 | 1.352(11) |
| N1A | C12A | 1.367(12) | N5 | C9 | 1.458(11) |
| N1A | C13A | 1.460(11) | N5 | C45 | 1.462(11) |
| N2A | C2A | 1.362(12) | N6 | C10 | 1.349(11) |
| N2A | C3A | 1.456(10) | N6 | C11 | 1.460(10) |
| N2A | C17A | 1.443(10) | N6 | C49 | 1.456(10) |
| N3A | C4A | 1.359(11) | N21 | C19 | 1.436(10) |
| N3A | C5A | 1.437(10) | N21 | C21 | 1.342(12) |
| N3A | C29A | 1.444(11) | N22 | C20 | 1.356(12) |
| N4A | C6A | 1.340(11) | N22 | C21 | 1.408(12) |
| N4A | C7A | 1.478(10) | N22 | C22 | 1.444(11) |
| N4A | C33A | 1.454(11) | N41 | C35 | 1.428(10) |
| N5A | C8A | 1.345(11) | N41 | C37 | 1.342(12) |
| N5A | C9A | 1.448(10) | N42 | C36 | 1.349(12) |
| N5A | C45A | 1.477(10) | N42 | C37 | 1.415(12) |
| N6A | C10A | 1.341(11) | N42 | C38 | 1.462(12) |
| N6A | C11A | 1.490(10) | N61 | C51 | 1.438(10) |
| N6A | C49A | 1.457(10) | N61 | C53 | 1.338(12) |
| N21A | C19A | 1.440(10) | N62 | C52 | 1.354(12) |
| N21A | C21A | 1.358(12) | N62 | C53 | 1.404(12) |
| N22A | C20A | 1.364(13) | N62 | C54 | 1.444(11) |
| N22A | C21A | 1.390(12) | C1 | C2 | 1.530(13) |
| N22A | C22A | 1.457(11) | C3 | C4 | 1.519(12) |
| N41A | C35A | 1.442(10) | C3 | C18 | 1.516(11) |
| N41A | C37A | 1.359(12) | C5 | C6 | 1.487(12) |
| N42A | C36A | 1.360(11) | C7 | C8 | 1.507(13) |
| N42A | C37A | 1.418(12) | C7 | C34 | 1.512(11) |
| N42A | C38A | 1.462(11) | C9 | C10 | 1.500(13) |
| N61A | C51A | 1.444(10) | C11 | C12 | 1.547(12) |
| N61A | C53A | 1.357(12) | C11 | C50 | 1.549(11) |
| N62A | C52A | 1.354(12) | C13 | C14 | 1.521(12) |
| N62A | C53A | 1.419(12) | C14 | C15 | 1.502(12) |
| N62A | C54A | 1.457(11) | C14 | C16 | 1.521(12) |
| C1A | C2A | 1.510(12) | C17 | C19 | 1.510(12) |
| C3A | C4A | 1.509(13) | C18 | C19 | 1.544(12) |
| C3A | C18A | 1.541(11) | C19 | C20 | 1.514(14) |
| C5A | C6A | 1.528(13) | C22 | C23 | 1.54(2) |
| C7A | C8A | 1.555(12) | C22 | C0AA | 1.501(15) |
| C7A | C34A | 1.516(11) | C23 | C24 | 1.3900 |
| C9A | C10A | 1.521(12) | C23 | C28 | 1.3900 |
| C11A | C12A | 1.521(12) | C24 | C25 | 1.3900 |
| C11A | C50A | 1.551(11) | C25 | C26 | 1.3900 |
| C13A | C14A | 1.526(12) | C26 | C27 | 1.3900 |
| C14A | C15A | 1.500(12) | C27 | C28 | 1.3900 |
| C14A | C16A | 1.539(13) | C29 | C30 | 1.510(12) |
| C17A | C19A | 1.528(11) | C30 | C31 | 1.522(12) |
| C18A | C19A | 1.506(11) | C30 | C32 | 1.503(11) |
| C19A | C20A | 1.529(13) | C33 | C35 | 1.527(11) |
| C22A | C23A | 1.56(3) | C34 | C35 | 1.531(12) |
| C22A | C23B | 1.509(11) | C35 | C36 | 1.521(14) |
| C23A | C24A | 1.3900 | C38 | C39 | 1.476(12) |
| C23A | C28A | 1.3900 | C39 | C40 | 1.387(12) |
| C24A | C25A | 1.3900 | C39 | C44 | 1.369(12) |
| C25A | C26A | 1.3900 | C40 | C41 | 1.383(12) |
| C26A | C27A | 1.3900 | C41 | C42 | 1.381(14) |
| C27A | C28A | 1.3900 | C42 | C43 | 1.382(13) |
| C29A | C30A | 1.524(12) | C43 | C44 | 1.400(13) |
| C30A | C31A | 1.531(13) | C45 | C46 | 1.523(12) |
| C30A | C32A | 1.498(13) | C46 | C47 | 1.535(12) |
| C33A | C35A | 1.539(11) | C46 | C48 | 1.530(12) |
| C34A | C35A | 1.527(12) | C49 | C51 | 1.524(12) |
| C35A | C36A | 1.523(13) | C50 | C51 | 1.521(12) |
| C38A | C39A | 1.507(12) | C51 | C52 | 1.515(13) |
| C39A | C40A | 1.375(13) | C54 | C55 | 1.481(13) |
| C39A | C44A | 1.373(12) | C55 | C56 | 1.397(13) |
| C40A | C41A | 1.379(13) | C55 | C60 | 1.388(13) |
| C41A | C42A | 1.378(14) | C56 | C57 | 1.396(15) |
| C42A | C43A | 1.355(15) | C57 | C58 | 1.355(15) |
| C43A | C44A | 1.408(14) | C58 | C59 | 1.374(15) |
| C45A | C46A | 1.495(12) | C59 | C60 | 1.378(14) |
| C46A | C47A | 1.520(12) | O3S | C6S | 1.464(12) |
| C46A | C48A | 1.540(11) | O3S | C7S | 1.282(14) |
| C49A | C51A | 1.517(11) | O4S | C7S | 1.281(16) |
| C50A | C51A | 1.529(11) | C5S | C6S | 1.522(16) |
| C51A | C52A | 1.527(13) | C7S | C8S | 1.470(18) |
| C54A | C55A | 1.510(13) | O1S | C2S | 1.440(14) |
| C55A | C56A | 1.353(12) | O1S | C3S | 1.329(13) |
| C55A | C60A | 1.415(13) | O2S | C3S | 1.200(14) |
| C56A | C57A | 1.383(13) | C1S | C2S | 1.32(12) |
| C57A | C58A | 1.351(14) | C2S | C1T | 1.46(3) |
| C58A | C59A | 1.388(14) | C3S | C4S | 1.480(16) |
| C59A | C60A | 1.404(13) | C0AA | C1AA | 1.3900 |
| O1 | C12 | 1.229(10) | C0AA | C5AA | 1.3900 |
| O2 | C2 | 1.240(11) | C1AA | C2AA | 1.3900 |
| O3 | C4 | 1.264(11) | C2AA | C3AA | 1.3900 |
| O4 | C6 | 1.240(11) | C3AA | C4AA | 1.3900 |
| O5 | C8 | 1.262(11) | C4AA | C5AA | 1.3900 |
| O6 | C10 | 1.233(10) | C23B | C24B | 1.3900 |
| O21 | C21 | 1.205(12) | C23B | C28B | 1.3900 |
| O22 | C20 | 1.217(11) | C24B | C25B | 1.3900 |
| O41 | C37 | 1.236(12) | C25B | C26B | 1.3900 |
| O42 | C36 | 1.203(11) | C26B | C27B | 1.3900 |
| O61 | C53 | 1.210(11) | C27B | C28B | 1.3900 |
| O62 | C52 | 1.220(11) | | | |

TABLE 3
Bond Angles of QPM-3.

| Atom | Atom | Atom | Angle/° | Atom | Atom | Atom | Angle/° |
|---|---|---|---|---|---|---|---|
| C1A | N1A | C13A | 116.5(8) | C9 | N5 | C45 | 120.4(8) |
| C12A | N1A | C1A | 124.0(8) | C10 | N6 | C11 | 126.8(8) |
| C12A | N1A | C13A | 118.9(8) | C10 | N6 | C49 | 123.5(8) |
| C2A | N2A | C3A | 116.3(8) | C49 | N6 | C11 | 109.7(7) |
| C2A | N2A | C17A | 125.1(8) | C21 | N21 | C19 | 114.4(8) |
| C17A | N2A | C3A | 114.1(8) | C20 | N22 | C21 | 110.6(9) |
| C4A | N3A | C5A | 122.0(8) | C20 | N22 | C22 | 126.6(10) |
| C4A | N3A | C29A | 119.3(8) | C21 | N22 | C22 | 122.7(11) |
| C5A | N3A | C29A | 118.5(8) | C37 | N41 | C35 | 111.0(9) |
| C6A | N4A | C7A | 125.6(8) | C36 | N42 | C37 | 110.7(10) |
| C6A | N4A | C33A | 121.2(8) | C36 | N42 | C38 | 125.8(10) |
| C33A | N4A | C7A | 111.8(7) | C37 | N42 | C38 | 123.4(11) |
| C8A | N5A | C9A | 125.0(9) | C53 | N61 | C51 | 114.2(9) |
| C8A | N5A | C45A | 118.2(7) | C52 | N62 | C53 | 111.8(10) |
| C9A | N5A | C45A | 116.8(8) | C52 | N62 | C54 | 124.5(10) |
| C10A | N6A | C11A | 125.9(8) | C53 | N62 | C54 | 121.5(11) |
| C10A | N6A | C49A | 119.9(8) | N1 | C1 | C2 | 113.6(8) |
| C49A | N6A | C11A | 112.8(7) | O2 | C2 | N2 | 120.5(9) |
| C21A | N21A | C19A | 112.7(8) | O2 | C2 | C1 | 121.6(11) |
| C20A | N22A | C21A | 113.3(9) | N2 | C2 | C1 | 117.6(10) |
| C20A | N22A | C22A | 123.1(11) | N2 | C3 | C4 | 110.3(7) |
| C21A | N22A | C22A | 123.4(11) | N2 | C3 | C18 | 101.4(7) |
| C37A | N41A | C35A | 113.2(8) | C18 | C3 | C4 | 115.1(8) |
| C36A | N42A | C37A | 112.9(9) | O3 | C4 | N3 | 120.6(8) |
| C36A | N42A | C38A | 124.7(9) | O3 | C4 | C3 | 118.1(10) |
| C37A | N42A | C38A | 122.4(10) | N3 | C4 | C3 | 121.2(10) |
| C53A | N61A | C51A | 114.5(8) | N3 | C5 | C6 | 112.8(8) |
| C52A | N62A | C53A | 112.5(9) | O4 | C6 | N4 | 121.9(8) |
| C52A | N62A | C54A | 124.5(10) | O4 | C6 | C5 | 120.1(11) |
| C53A | N62A | C54A | 122.8(10) | N4 | C6 | C5 | 118.0(10) |
| N1A | C1A | C2A | 113.8(8) | N4 | C7 | C8 | 110.8(8) |
| O2A | C2A | N2A | 122.8(9) | N4 | C7 | C34 | 103.4(7) |
| O2A | C2A | C1A | 121.6(11) | C8 | C7 | C34 | 113.0(7) |
| N2A | C2A | C1A | 115.5(10) | O5 | C8 | N5 | 120.6(9) |
| N2A | C3A | C4A | 114.4(8) | O5 | C8 | C7 | 118.2(10) |
| N2A | C3A | C18A | 101.3(7) | N5 | C8 | C7 | 121.2(10) |
| C4A | C3A | C18A | 109.1(8) | N5 | C9 | C10 | 115.4(8) |
| O3A | C4A | N3A | 119.7(9) | O6 | C10 | N6 | 121.3(9) |
| O3A | C4A | C3A | 121.5(10) | O6 | C10 | C9 | 120.7(9) |
| N3A | C4A | C3A | 118.6(10) | N6 | C10 | C9 | 117.9(10) |
| N3A | C5A | C6A | 112.9(8) | N6 | C11 | C12 | 111.3(8) |
| O4A | C6A | N4A | 120.3(10) | N6 | C11 | C50 | 105.1(7) |
| O4A | C6A | C5A | 121.1(10) | C12 | C11 | C50 | 109.3(7) |
| N4A | C6A | C5A | 118.5(10) | O1 | C12 | N1 | 122.3(9) |
| N4A | C7A | C8A | 111.0(8) | O1 | C12 | C11 | 118.7(9) |
| N4A | C7A | C34A | 103.8(7) | N1 | C12 | C11 | 119.0(10) |
| C34A | C7A | C8A | 110.9(7) | N1 | C13 | C14 | 117.0(7) |
| O5A | C8A | N5A | 123.8(9) | C13 | C14 | C16 | 111.9(7) |
| O5A | C8A | C7A | 119.5(10) | C15 | C14 | C13 | 113.7(8) |
| N5A | C8A | C7A | 116.5(10) | C15 | C14 | C16 | 110.7(8) |
| N5A | C9A | C10A | 113.8(8) | N2 | C17 | C19 | 105.0(8) |
| O6A | C10A | N6A | 123.6(8) | C3 | C18 | C19 | 106.3(7) |
| O6A | C10A | C9A | 119.2(10) | N21 | C19 | C17 | 116.6(8) |
| N6A | C10A | C9A | 117.1(10) | N21 | C19 | C18 | 114.1(8) |
| N6A | C11A | C12A | 113.4(8) | N21 | C19 | C20 | 100.0(8) |
| N6A | C11A | C50A | 101.7(7) | C17 | C19 | C18 | 103.4(7) |
| C12A | C11A | C50A | 108.9(7) | C17 | C19 | C20 | 109.0(8) |
| O1A | C12A | N1A | 123.3(9) | C20 | C19 | C18 | 114.2(8) |
| O1A | C12A | C11A | 120.3(11) | O22 | C20 | N22 | 124.2(10) |
| N1A | C12A | C11A | 116.0(10) | O22 | C20 | C19 | 127.2(11) |
| N1A | C13A | C14A | 112.4(7) | N22 | C20 | C19 | 108.5(9) |
| C13A | C14A | C16A | 109.1(8) | O21 | C21 | N21 | 129.3(12) |
| C15A | C14A | C13A | 113.2(8) | O21 | C21 | N22 | 124.5(13) |
| C15A | C14A | C16A | 111.7(9) | N21 | C21 | N22 | 106.2(10) |
| N2A | C17A | C19A | 100.9(7) | N22 | C22 | C23 | 116.7(15) |
| C19A | C18A | C3A | 105.3(7) | N22 | C22 | C0AA | 112.0(12) |
| N21A | C19A | C17A | 112.6(8) | C24 | C23 | C22 | 119.1(17) |
| N21A | C19A | C18A | 112.8(8) | C24 | C23 | C28 | 120.0 |
| N21A | C19A | C20A | 101.9(8) | C28 | C23 | C22 | 120.7(17) |
| C17A | C19A | C20A | 111.5(9) | C23 | C24 | C25 | 120.0 |
| C18A | C19A | C17A | 101.9(7) | C26 | C25 | C24 | 120.0 |
| C18A | C19A | C20A | 116.5(8) | C25 | C26 | C27 | 120.0 |
| O22A | C20A | N22A | 129.5(12) | C26 | C27 | C28 | 120.0 |
| O22A | C20A | C19A | 125.0(13) | C27 | C28 | C23 | 120.0 |
| N22A | C20A | C19A | 105.4(10) | N3 | C29 | C30 | 114.8(7) |
| O21A | C21A | N21A | 129.8(11) | C29 | C30 | C31 | 111.9(7) |
| O21A | C21A | N22A | 124.1(12) | C32 | C30 | C29 | 109.0(8) |
| N21A | C21A | N22A | 106.1(10) | C32 | C30 | C31 | 112.5(8) |
| N22A | C22A | C23A | 101.8(19) | N4 | C33 | C35 | 103.2(8) |
| N22A | C22A | C23B | 115.2(8) | C7 | C34 | C35 | 104.0(7) |
| C24A | C23A | C22A | 131(2) | N41 | C35 | C33 | 114.8(8) |
| C24A | C23A | C28A | 120.0 | N41 | C35 | C34 | 117.2(8) |
| C28A | C23A | C22A | 109(2) | N41 | C35 | C36 | 103.0(8) |
| C25A | C24A | C23A | 120.0 | C33 | C35 | C34 | 101.6(7) |
| C24A | C25A | C26A | 120.0 | C36 | C35 | C33 | 109.0(8) |
| C27A | C26A | C25A | 120.0 | C36 | C35 | C34 | 111.3(9) |
| C28A | C27A | C26A | 120.0 | O42 | C36 | N42 | 125.1(11) |
| C27A | C28A | C23A | 120.0 | O42 | C36 | C35 | 128.5(12) |
| N3A | C29A | C30A | 115.2(7) | N42 | C36 | C35 | 106.3(10) |
| C29A | C30A | C31A | 109.6(8) | O41 | C37 | N41 | 127.9(14) |
| C32A | C30A | C29A | 113.0(8) | O41 | C37 | N42 | 123.9(13) |
| C32A | C30A | C31A | 110.9(8) | N41 | C37 | N42 | 108.1(10) |
| N4A | C33A | C35A | 103.1(7) | N42 | C38 | C39 | 113.5(8) |
| C7A | C34A | C35A | 103.5(7) | C40 | C39 | C38 | 124.0(11) |
| N41A | C35A | C33A | 113.4(8) | C44 | C39 | C38 | 118.4(11) |
| N41A | C35A | C34A | 117.6(8) | C44 | C39 | C40 | 117.6(9) |
| N41A | C35A | C36A | 102.0(7) | C41 | C40 | C39 | 121.3(9) |
| C34A | C35A | C33A | 102.5(7) | C42 | C41 | C40 | 120.5(10) |
| C36A | C35A | C33A | 108.1(8) | C41 | C42 | C43 | 119.2(10) |
| C36A | C35A | C34A | 113.3(8) | C42 | C43 | C44 | 119.2(10) |
| O42A | C36A | N42A | 126.5(9) | C39 | C44 | C43 | 122.1(9) |
| O42A | C36A | C35A | 127.7(11) | N5 | C45 | C46 | 113.4(7) |
| N42A | C36A | C35A | 105.8(9) | C45 | C46 | C47 | 108.1(8) |
| O41A | C37A | N41A | 128.9(11) | C45 | C46 | C48 | 112.0(7) |
| O41A | C37A | N42A | 125.9(11) | C48 | C46 | C47 | 112.5(8) |
| N41A | C37A | N42A | 105.2(10) | N6 | C49 | C51 | 105.3(7) |
| N42A | C38A | C39A | 114.2(7) | C51 | C50 | C11 | 106.5(7) |
| C40A | C39A | C38A | 120.4(11) | N61 | C51 | C49 | 113.5(8) |
| C44A | C39A | C38A | 120.9(12) | N61 | C51 | C50 | 115.5(9) |
| C44A | C39A | C40A | 118.6(9) | N61 | C51 | C52 | 100.7(8) |
| C39A | C40A | C41A | 122.2(11) | C50 | C51 | C49 | 103.0(7) |
| C42A | C41A | C40A | 117.9(11) | C52 | C51 | C49 | 109.4(8) |
| C43A | C42A | C41A | 121.9(11) | C52 | C51 | C50 | 115.0(9) |
| C42A | C43A | C44A | 119.1(10) | O62 | C52 | N62 | 126.0(11) |
| C39A | C44A | C43A | 120.2(10) | O62 | C52 | C51 | 126.6(11) |
| N5A | C45A | C46A | 115.4(7) | N62 | C52 | C51 | 107.4(10) |
| C45A | C46A | C47A | 110.7(7) | O61 | C53 | N61 | 128.5(12) |
| C45A | C46A | C48A | 110.4(7) | O61 | C53 | N62 | 125.6(13) |
| C47A | C46A | C48A | 111.4(8) | N61 | C53 | N62 | 105.9(10) |
| N6A | C49A | C51A | 104.2(7) | N62 | C54 | C55 | 117.6(8) |
| C51A | C50A | C11A | 107.0(7) | C56 | C55 | C54 | 121.0(12) |
| N61A | C51A | C49A | 115.3(8) | C60 | C55 | C54 | 122.6(12) |
| N61A | C51A | C50A | 115.7(8) | C60 | C55 | C56 | 116.3(10) |
| N61A | C51A | C52A | 100.4(8) | C57 | C56 | C55 | 121.4(10) |
| C49A | C51A | C50A | 102.6(7) | C58 | C57 | C56 | 120.1(11) |
| C49A | C51A | C52A | 111.5(8) | C57 | C58 | C59 | 119.9(11) |
| C52A | C51A | C50A | 111.7(8) | C58 | C59 | C60 | 120.0(10) |
| O62A | C52A | N62A | 127.1(10) | C59 | C60 | C55 | 122.2(10) |
| O62A | C52A | C51A | 125.7(11) | C7S | O3S | C6S | 119.8(12) |
| N62A | C52A | C51A | 107.1(9) | O3S | C6S | C5S | 108.1(10) |
| O61A | C53A | N61A | 128.4(11) | O3S | C7S | C8S | 118.6(15) |
| O61A | C53A | N62A | 127.0(11) | O4S | C7S | O3S | 119.4(18) |
| N61A | C53A | N62A | 104.7(10) | O4S | C7S | C8S | 122.0(13) |
| N62A | C54A | C55A | 112.0(8) | C3S | O1S | C2S | 117.0(10) |
| C56A | C55A | C54A | 123.0(11) | O1S | C2S | C1T | 110.3(15) |
| C56A | C55A | C60A | 118.8(9) | C1S | C2S | O1S | 128(5) |
| C60A | C55A | C54A | 118.1(12) | O1S | C3S | C4S | 113.3(13) |
| C55A | C56A | C57A | 122.7(10) | O2S | C3S | O1S | 122.3(13) |
| C58A | C57A | C56A | 119.1(10) | O2S | C3S | C4S | 124.4(13) |
| C57A | C58A | C59A | 120.9(10) | C1AA | C0AA | C22 | 120.3(11) |
| C58A | C59A | C60A | 120.0(10) | C1AA | C0AA | C5AA | 120.0 |
| C59A | C60A | C55A | 118.4(10) | C5AA | C0AA | C22 | 119.6(11) |
| C1 | N1 | C13 | 117.0(7) | C2AA | C1AA | C0AA | 120.0 |
| C12 | N1 | C1 | 123.0(9) | C1AA | C2AA | C3AA | 120.0 |
| C12 | N1 | C13 | 119.9(8) | C4AA | C3AA | C2AA | 120.0 |
| C2 | N2 | C3 | 120.3(7) | C3AA | C4AA | C5AA | 120.0 |
| C2 | N2 | C17 | 125.4(9) | C4AA | C5AA | C0AA | 120.0 |
| C17 | N2 | C3 | 113.5(8) | C24B | C23B | C22A | 116.7(7) |
| C4 | N3 | C5 | 121.2(8) | C24B | C23B | C28B | 120.0 |
| C4 | N3 | C29 | 121.5(7) | C28B | C23B | C22A | 123.3(7) |
| C29 | N3 | C5 | 117.1(8) | C23B | C24B | C25B | 120.0 |
| C6 | N4 | C7 | 126.2(8) | C26B | C25B | C24B | 120.0 |
| C6 | N4 | C33 | 120.9(8) | C27B | C26B | C25B | 120.0 |
| C33 | N4 | C7 | 111.8(7) | C28B | C27B | C26B | 120.0 |
| C8 | N5 | C9 | 120.7(8) | C27B | C28B | C23B | 120.0 |
| C8 | N5 | C45 | 118.9(8) | | | | |

The diversity of cis and trans amides seen in the crystal structure supports the idea that unbound macrocycles are highly disordered. When comparing the unbound vs. bound state, the core structure of the macrocycle goes through a ring inversion between the unbound crystal structure and the metal bound NMIR structure. This ordering of macrocycles upon cation binding is consistent with simulation studies by Hurley et al., in which ROE-restrained modeling (Voelz V A et al., 2014, Comput. Chem., 35:2215) and directly observed cation association dynamics confirms an ordered, predominantly trans-amide, macrocycle conformation.

In summary, this work introduces Fmoc-protected Q-Pro amino acids, which display two functional groups that are added after the stereochemistry has been set. After developing numerous inventive synthetic steps (i.e., performing various studies to identify optimal protecting groups and optimal reaction conditions, such as using microwave reactor and longer reaction times), these derivatives are synthesized on multigram scale with a single reversed-phase purification, and are readily incorporated into standard Fmoc-SPPS. The resultant peptidomimetics are disordered in the absence of a metal cation, as evidenced by the NMR and the racemic crystal structure. In the presence of a metal cation, however, these macrocycles adopt a similar structure regardless of the functional groups appended to the molecules. Addition of a second functional group to the hydantoin amide position converts the proline ring from a Cγ-endo to a Cγ-exo conformation, most likely due to steric strain caused by addition of this functional group. Additional work is focused on application of these new amino acids towards catalytic and materials research.

In conclusion, Example 1 introduces the efficient synthesis of highly preorganized Q-Pro amino acids, which display two functional groups that are added after the stereochemistry has been defined. After developing numerous inventive synthetic steps (i.e., performing various studies to identify optimal protecting groups and optimal reaction conditions, such as using microwave reactor and longer reaction times), these amino acids have been synthesized on multigram scale with a single purification in good yields (58-78% over 3 steps). Synthesis of eight QPMs was achieved through standard Fmoc-SPPS and peptoid chemistry. It was shown that QPM are disordered in the absence of a metal cation, as evidenced by NMR and a crystal structure of QPM-3 obtained through racemic crystallization. However, in the presence of a metal cation, these macrocycles adopt an ordered, uniform core structure regardless of the functional groups on the macrocycles. Late stage functionalization of the Q-Pro macrocycles via alkylation chemistry allows the installation of a reactive functional group as the final step in a synthesis. Furthermore, the addition of this second functional group to the hydantoin amide position (R₂) converts the proline ring from a Cγ-endo to a Cγ-exo conformation, most likely due to the steric crowding caused by the addition of this functional group.

The materials and methods employed in these experiments are now described.

General Procedure 1—Synthesis of Enhanced Proline Derivatives from Compound 1

Step 1:

To a stirred mixture of compound 1 in DMF (100 mM) 0.75-0.88 equiv. (equivalents dependent on salt content of the specific batch of compound 1, which can be determined utilizing an internal standard) of a halide along with 1.5 equiv. of K₂CO₃ was added. The reaction proceeded at room temperature for 2-24 hours, at which time 1.5 equiv. of an allyl or benzyl halide and 1.5 equiv. of K₂CO₃ was added to the reaction mixture and stirred overnight. The reaction was diluted with four times the reaction volume of EtOAC and washed with water, saturated ammonium chloride solution, and brine. The organic layer was dried with Na₂SO₄, and concentrated in vacuo to yield the foamy off-white to yellow solids of compound 2 (compounds have been reported previously).

Step 2:

These compounds were deprotected using a 1:1 mixture of DCM/(33% HBr in AcOH) for 30 minutes, and the solvent was removed in vacuo. The deprotected amino acid was dissolved in DMF and free based with 3 equiv. of DIPEA, after which 1.1 equiv. of Fmoc-OSu was added to the reaction. This was stirred for 2 hours, and the progress was checked via HPLC-MS. Upon completion, the reaction was diluted with EtOAc, washed with ammonium chloride and brine, dried with Na₂SO₄, concentrated in vacuo to yield dark yellow solids, which were purified by reverse phase flash chromatography (5-95% acetonitrile in water, 0.1% formic acid). The fractions were combined, the acetonitrile was removed in vacuo, and the product was extracted from the residual aqueous mixture with EtOAc, rinsed with brine, and dried over Na₂SO₄. The EtOAc was removed in vacuo to yield off white Fmoc protected amino acids Q-Pro 1-12.

### Characterization Results

### QPro 1: (5S,8S)-7-((9H-fluoren-9-yl)methoxy)carbonyl)-3-benzyl-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.88 equiv. benzyl bromide to recover 1.44 g pure QPro 1 (75%).

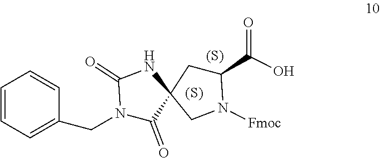

¹H NMR (500 MHz, CDCl₃, rotamers present) 2.22 (1H, dd, J=13.9, 2.2), 2.88 (1H, m), 3.94 (1H, m), 3.97 (1H, d, J=6.3), 4.21 (1H, m), 4.37 (1.5H, m), 4.55 (1H, m), 4.65 (0.5H, d, J=9.5), 4.71 (2H, m), 7.33 (101H, m), 7.58 (4H, m), 8.58 (1H, s, rotameric); ¹³C NMR (125 MHz, CDCl₃, rotamers present) 39.8, 40.8, 42.8, 42.9, 47.0, 47.1, 55.7, 56.4, 57.7, 58.2, 66.1, 67.2, 68.1, 76.8, 77.1, 77.3, 120.0, 125.0, 125.1, 127.0, 127.2, 127.7, 127.8, 128.4, 128.5, 128.6, 128.91, 128.93, 135.27, 141.2, 141.28, 141.29, 141.4, 143.5, 143.8, 143.9, 153.5, 154.0, 158.2, 158.3, 172.1, 172.3, 176.3, 176.4; HRMS-ESI: m/z calcd for C₂₉H₂₅N₃O₆Na (M+Na)⁺ 534.1636, found 544.1645.

### QPro 2: (5S,8S)-7-((9H-fluoren-9-yl)methoxy)carbonyl)-3-(cyclopropylmethyl)-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 1.25 equiv. of (bromomethyl)cyclopropane to recover 1.40 g pure QPro 2 (75%).

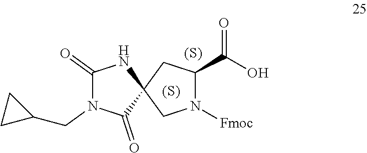

¹H NMR (500 MHz, CDCl₃, rotamers present) 0.35 (2H, m), 0.52 (2H, m), 1.18 (1H, m), 2.26 (1H, d, J=14.2), 2.89 (1H, m), 3.40 (1H, d, J=7.3), 3.43 (1H, dd, J=7.4, 2.7), 3.98 (2H, d, J=14.5), 4.22 (1H, q, J=7.0), 4.37 (1.5H, m), 4.51 (0.5H, dd, J=10.7, 6.6), 4.65 (1H, d, J=9.8, rotameric), 7.30 (5H), 7.60 (4H, m), 8.58 (1H, s, rotameric); ¹³C NMR (125 MHz, CDCl₃, rotamers present) 3.9, 10.1, 39.9, 40.9, 44.0, 44.1, 47.0, 47.2, 55.8, 56.5, 57.8, 58.3, 66.1, 67.1, 68.17, 68.23, 120.02, 120.05, 125.0, 125.1, 125.2, 127.1, 127.15, 127.16, 127.2, 127.7, 127.8, 128.5, 141.2, 141.3, 141.4, 143.6, 143.8, 143.9, 153.6, 154.1, 158.7, 158.8, 172.5, 172.7, 176.4, 176.5; HRMS-ESI: m/z calcd for C₂₆H₂₅N₃O₆Na (M+Na)⁺ 498.1636, found 498.1624.

### QPro 3: (5S,8S)-7-(((9H-fluoren-9-yl)methoxy)carbonyl)-3-(4-methoxybenzyl)-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.88 equiv. 4-methoxybenzylchloride to recover 1.35 g pure QPro 3 (70%).

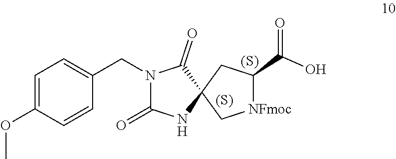

¹H NMR (500 MHz, CDCl₃, rotamers present) 2.18 (1H, d, J=14.0), 2.85 (1H, dd, J=9.8, 4.3), 3.78 (3H, s), 3.92 (2H, m), 4.19 (1H, m), 4.36 (2H, m, rotameric to 4.49), 4.62 (2H, m, rotameric to 4.53), 6.85 (2H, dd, J=10.4, 8.9), 7.28 (6H, m), 7.49 (1H, d, J 7.5), 7.57 (1H, d, J=8.2), 7.70 (2H, m), 8.45 (1H, s); ¹³C NMR (125 MHz, CDCl₃, rotamers present) 31.5, 36.6, 39.7, 40.8, 42.27, 42.31, 46.9, 47.1, 55.2, 55.3, 55.6, 56.4, 57.7, 58.3, 66.0, 67.1, 68.10, 68.16, 114.0, 114.2, 119.94, 119.96, 119.98, 124.9, 125.00, 125.05, 125.08, 127.0, 127.08, 127.11, 127.2, 127.5, 127.6, 127.72, 127.75, 127.76, 128.9, 130.03, 130.05, 130.2, 141.17, 141.22, 141.24, 141.3, 143.5, 143.7, 153.5, 154.0, 158.0, 158.2, 159.5, 159.6, 162.8, 172.1, 172.2, 176.3, 176.4; HRMS-ESI: m/z calcd for C₃₀H₂₇N₃O₇Na (M+Na)⁺ 564.1741, found 564.1755.

### QPro 4: (5S,8S)-7-(((9H-fluoren-9-yl)methoxy)carbonyl)-3-benzyl-1-methyl-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.88 equiv. benzyl bromide, and 1.25 equiv. iodomethane to recover 1.60 g pure QPro 4 (78%).

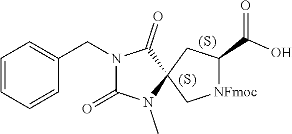

¹H NMR (500 MHz, CDCl₃ rotamers present) 2.36 (1H, dd, J=13.9, 7.5), 2.54 (1H, dd, J=13.7, 8.9) 2.67 (4H, m), 3.59 (1H, d, J=11.6), 3.78 (1H, d, J=11.6), 4.17 (1H, t, J=7.0), 4.37 (1H, d, J=7.0), 4.44 (1H, dd, rotameric, J=10.7, 7.0), 4.66 (2H,), 4.72 (1H, m), 7.31 (9H, m), 7.50 (2H, t, J=7.2), 7.72 (2H, m); ¹³C NMR (125 MHz, CDCl₃ or DMSO-d₆, rotamers present) 24.9, 25.0, 24.2, 31.8, 34.3, 35.6, 36.9, 42.6, 42.7, 46.9, 47.1, 49.6, 49.8, 57.8, 58.5, 66.1, 67.0, 68.3, 68.4, 119.9, 120.1, 124.9, 125.0, 125.09, 125.13, 127.1, 127.7, 127.80, 127.84, 128.11, 128.15, 128.45, 128.50, 128.57, 128.8, 135.7, 141.23, 141.25, 143.4, 143.5, 143.6, 155.1, 155.2, 163.5, 173.7, 174.0, 174.6; HRMS-ESI: m/z calcd for C₃₀H₂₇N₃O₆Na (M+Na)⁺ 548.1792, found 548.1784.

### QPro 5: (5S,8S)-7-(((9H-fluoren-9-yl)methoxy)carbonyl)-3-allyl-1-(4-bromobenzyl)-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S), 0.88 equiv. allyl bromide, and 1.25 equiv. 4-bromobenzyl bromide to recover 1.42 g pure QPro 5 (58%).

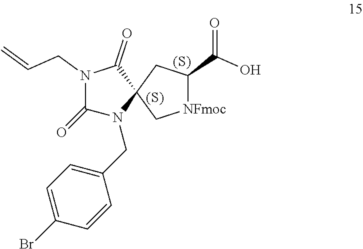

¹H NMR (500 MHz, CDCl₃, rotamers present) 2.39 (1H, dd, J=13.6, 7.8), 2.52 (1H, dd, J=13.7, 8.9), 3.40 (1H, d, J=11.8), 3.64 (1H, d, J=11.7), 4.18 (3H, m), 4.32 (1H, m), 4.40 (2H, m), 4.52 (1H, m), 4.67 (2H, m), 5.24 (2H, m), 5.87 (1H, m), 7.13 (2H, m), 7.26 (2H, m), 7.44 (6H, m), 7.72 (2H, m); ¹³C NMR (125 MHz, CDCl₃ or DMSO-d₆, rotamers present) 14.2, 34.6, 41.3, 42.5, 46.8, 50.1, 58.1, 67.1, 68.1, 68.4, 118.7, 120.1, 122.1, 124.8, 124.9, 127.14, 127.17, 127.8, 127.9, 129.0, 129.2, 129.7, 130.6, 130.8, 131.7, 132.2, 135.9, 136.5, 141.22, 141.25, 143.3, 143.4, 155.4, 155.37, 155.39, 155.45, 173.5, 173.9; HRMS-ESI: m/z calcd for C₃₂H₂₈BrN₃O₆Na (M+Na)⁺ 652.1024, found 652.1024.

### QPro 6: (5S,8S)-7-(((9H-fluoren-9-yl)methoxy)carbonyl)-1,3-bis(2-(benzyloxy)-2-oxoethyl)-2,4-dioxo-1,3,7-triazaspiro[4.4]nonane-8-carboxylic acid

Using general procedure 1 with 2.33 g 1 (7-benzyl 8-(tert-butyl) (8S)-2,4-dioxo-1,3,7-triazaspiro[4.4]nonane-7,8-dicarboxylate) (2S, 4S) and 1.3 equiv. benzyl bromoacetate to recover 1.59 g pure QPro 6 (53%).

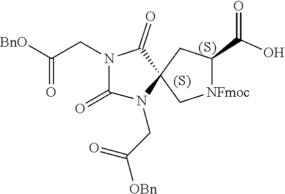

¹H NMR (500 MHz, CDCl₃, rotamers present) 2.35 (1H, dd, J=13.0, 7.5), 2.51 (1H, m), 3.56 (1H, d, J=11.3), 3.84 (4H, m), 4.15 (5H, m), 4.43 (1H, t, J=7.9), 5.02 (4H, m), 7.06 (2H, m), 7.23 (14H, m), 7.54 (2H, dd, J=13.6, 7.5); ¹³C NMR (125 MHz, CDCl₃ or DMSO-d₆, rotamers present) 35.7, 39.5, 41.0, 46.6, 50.5, 60.7, 67.4, 67.6, 67.9, 119.8, 125.1, 125.2, 127.0, 127.11, 127.6, 128.1, 128.28, 128.33, 128.50, 128.52, 128.59, 134.8, 135.0, 140.99, 141.03, 143.5, 143.7, 157.7, 155.6, 166.7, 169.1, 174.0, 177.7; HRMS-ESI: m/z calcd for C₄₀H₃₅N₃O₁₀Na (M+Na)⁺ 740.2215, found 740.2219.

### QPro 7: (5R,8S)-7-(((9H-fluoren-9-yl)methoxy)carbonyl)-3-benzyl-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.75 equiv. benzyl bromide to recover 1.34 pure QPro 7 (70%).

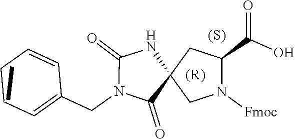

¹H NMR (500 MHz, DMSO-D₆, rotamers present) 2.27 (1H, dd, rotameric, J=13.1, 9.6), 2.65 (1H, ddd, rotameric, J=13.3, 8.0, 1.4), 3.63 (1H, dd, J=16.1, 11.3), 3.77 (1H, dd, rotameric, J=11.0, 1.3), 4.16 (1H, m), 4.29 (2H, m), 4.42 (0.5H, dd, J=9.5, 7.9), 4.59 (2H, s, rotameric), 4.63 (0.5H, m), 7.35 (9H, m), 7.66 (1H, d, J=7.6), 7.70 (1H, dd, J=7.6, 3.8), 7.89 (2H, m), 9.07 (1H, s, rotameric); ¹³C NMR (125 MHz, DMSO-D₆, rotamers present) 41.5, 46.5, 46.6, 55.6, 56.1, 57.8, 58.2, 59.8, 65.0, 65.7, 67.1, 67.6, 120.2, 125.2, 125.3, 127.15, 127.20, 127.38, 127.43, 127.77, 127.81, 136.41, 136.44, 140.6, 140.7, 143.5, 143.6, 143.7, 153.5, 153.7, 155.4, 172.3, 172.38, 172.44, 172.90; HRMS-ESI: m/z calcd for C₂₉H₂₅N₃O₆Na (M+Na)⁺ 534.1636, found 544.16439.

### QPro 8: (5R,8S)-7-(((9H-fluoren-9-yl)methoxy)carbonyl)-3-(cyclopropylmethyl)-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 1.25 equiv. of (bromomethyl)cyclopropane to recover 1.36 g pure QPro 8 (73%).

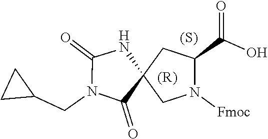

¹H NMR (500 MHz, DMSO-D₆, rotamers present) 0.27 (2H, m), 0.46 (2H, m), 1.06 (1H, m), 2.26 (1H, dd, rotameric, 13.2, 9.5), 2.60 (1H, ddd, rotameric J=13.3, 8.1, 1.6), 3.26 (2H, dd, J=6.9, 4.4), 3.62 (1H, m), 3.75 (1H, dd, rotameric, J=11.3, 1.3), 4.16 (1H, m), 4.29 (2H, s), 4.41 (1H, dd, rotameric, 9.5, 7.9), 7.34 (2H, m), 7.44 (2H, m), 7.67 (1H, d, J=7.3), 7.71 (1H, dd, J=7.4, 3.0), 7.91 (2H, d, rotameric J=7.6), 8.98 (1H, s, rotameric); ¹³C NMR (125 MHz, DMSO-D₆, rotamers present) 3.5, 9.9, 42.6, 46.4, 46.6, 55.6, 56.1, 57.8, 58.2, 64.8, 65.6 67.1, 67.5, 120.2, 125.2, 125.3, 125.32, 125.41, 127.2, 127.7, 127.76, 127.8, 140.62, 140.67, 140.71, 143.50, 143.57, 143.64, 143.7, 153.5, 153.7, 155.6, 155.7, 172.4, 172.5, 172.9; HRMS-ESI: m/z calcd for C₂₆H₂₅N₃O₆Na (M+Na)⁺ 498.1636, found 498.1641.

### QPro 9: (5R,8S)-7-(((9H-fluoren-9-yl)methoxy)carbonyl)-3-(4-methoxybenzyl)-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.75 equiv. of 4-methoxybenzyl chloride to recover 1.36 g pure QPro 9 (71%).

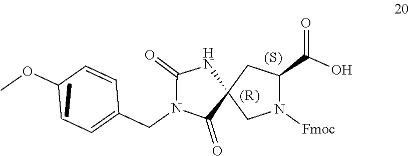

¹H NMR (500 MHz, DMSO-D₆, rotamers present) 2.25 (1H, dd, rotameric, J=13.2, 9.8), 2.62 (1H, ddd, rotameric, J=13.3, 7.9, 1.6), 3.62 (1H, d, rotameric, J=11.2), 3.75 (3H, s, rotameric), 3.84 (1H, dd, J=11.3, 1.6), 4.16 (1H, m), 4.29 (2H, s, rotameric), 4.41 (1H, dd, rotameric, J=9.5, 7.9), 4.51 (2H, s, rotameric), 6.91 (2H, m), 7.21 (2H, dd, J=8.7, 5.5), 7.39 (5H, m), 7.66 (1H, d, J=7.3), 7.71 (1H, dd, J=7.4, 3.3), 7.90 (2H, m), 9.03 (1H, s, rotameric); ¹³C NMR (125 MHz, DMSO-D₆, rotamers present) 46.9, 47.1, 55.5, 56.0, 56.5, 58.3, 58.6, 65.4, 66.1, 67.6, 70.0, 114.4, 120.7, 125.7, 125.73, 125.8, 125.9, 127.7, 128.2, 128.23, 128.3, 128.9, 128.94, 129.3, 129.34, 141.10, 141.14, 141.2, 144.1, 144.2, 154.0, 154.2, 155.9, 159.1, 172.7, 172.82, 172.84, 173.4; HRMS-ESI: m/z calcd for C₃₀H₂₇N₃O₇Na (M+Na)⁺ 564.1741, found 564.1751.

### QPro 10: (5R,8S)-7-(((9H-fluoren-9-yl)methoxy)carbonyl)-3-(4-methoxybenzyl)-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.75 equiv. benzyl bromide, and then 1.25 equiv. iodomethane to recover 1.49 g pure QPro 10 (73%).

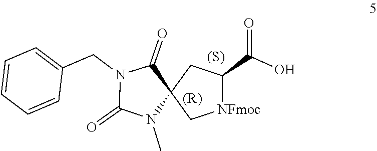

¹H NMR (500 MHz, CDCl₃, rotamers present) 2.46 (1H, dd, J=14.5, 9.3), 2.62 (1H, dd, J=14.6, 7.0), 2.76 (3H, s, 3.56 (1H, d, rotameric to 3.82 ppm, J=11.6), 3.77 (1H, d, J=11.9), 4.21 (1H, t, J=6.3), 4.33 (1H, m), 4.52 (2H, m), 4.62 (2H, s), 4.66 (1H, dd, J=9.2, 7.0), 7.31 (9H, m), 7.52 (2H, m), 7.71 (2H, m); ¹³C NMR (125 MHz, CDCl₃, rotamers present) 14.2, 25.43, 25.46, 25.55, 36.7, 37.9, 42.9, 43.0, 46.3, 47.1, 47.2, 53.1, 53.3, 57.8, 58.4, 67.5, 67.80, 67.82, 68.6, 72.9, 119.9, 120.00, 120.04, 120.2, 124.7, 124.8, 125.2, 127.1, 127.2, 127.4, 127.74, 127.77, 127.83, 127.87, 128.07, 128.14, 128.19, 128.4, 128.6, 128.76, 128.78, 135.5, 135.6, 141.20, 141.23, 141.27, 141.37, 141.42, 142.4, 143.25, 143.33, 143.6, 143.7, 153.7, 154.4, 154.69, 154.71, 168.7, 171.8, 172.2, 173.4, 174.1; HRMS-ESI. m/z calcd for C₃₀H₂₇N₃O₆Na (M+Na)⁺ 548.1792, found 548.1799.

### QPro 11: (5R,8S)-7-(((9H-fluoren-9-yl)methoxy)carbonyl)-3-benzyl-1-methyl-2,4-dioxo-1,3,7-triazaspiro[4.4]nonane-8-carboxylic acid

Using general procedure 1 with 1.50 g 1 (7-benzyl 8-(tert-butyl) (8S)-2,4-dioxo-1,3,7-triazaspiro[4.4]nonane-7,8-dicarboxylate) (2S, 4S) and 0.75 equiv. allyl bromide, and then 1.25 equiv. 4-bromobenzyl bromide to recover 1.37 g pure QPro 11 (56%).

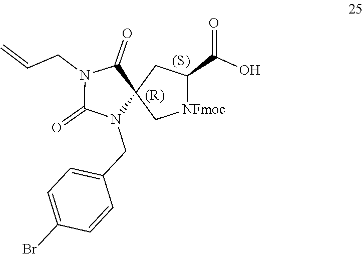

¹H NMR (500 MHz, CDCl₃, rotamers present) 2.39 (1H, ddd, J=14.6, 9.2, 1.2), 2.60 (1H, dd, J=14.6, 7.6), 3.45 (1H, dd, rotameric to 3.83, J=11.6, 1.2), 3.74 (1H, m), 4.11 (5H, m), 4.39 (1H, m), 4.54 (2H, m), 5.22 (2H, m), 5.84 (1H, m), 7.03 (2H, d, J=8.2), 7.35 (6H, m), 7.48 (2H, m), 7.73 (2H, t, J=8.4); ¹³C NMR (125 MHz, CDCl₃ or DMSO-d₆, rotamers present) 14.2, 37.5, 41.7, 43.5, 46.9, 47.0, 54.1, 58.3, 67.8, 69.2, 118.57, 118.59, 119.99, 120.04, 120.06, 122.1, 124.95, 124.99, 127.0, 127.10, 127.14, 127.16, 127.21, 127.6, 127.8, 127.9, 128.9, 129.0, 130.5, 130.6, 132.0, 132.1, 135.6, 141.29, 141.3, 143.3, 143.4, 153.9, 155.4, 171.2, 173.4; HRMS-ESI: m/z calcd for C₃₂H₂₈BrN₃O₆Na (M+Na)⁺ 652.1024, found 652.1029.

### QPro 12: (5R,8S)-7-(((9H-fluoren-9-yl)methoxy)carbonyl)-3-allyl-1-(4-bromobenzyl)-2,4-dioxo-1,3,7-triazaspiro[4.4]nonane-8-carboxylic acid

Synthesized with QPro 6, separated by column chromatography to recover 590 mg pure QPro 12 (53%).

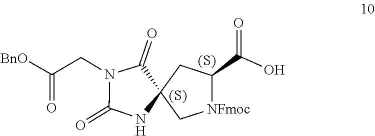

¹H NMR (500 MHz, CDCl₃ or DMSO-d₆) 2.26 (1H, d, J=13.7), 2.87 (1H, td, J=13.6, 9.8), 3.98 (2H, d, J=5.8), 4.20 (1H, m), 4.36 (4H, m), 4.55 (1H, m), 5.19 (2H, d, J=9.2), 7.28 (2H, m), 7.34 (7H, m), 7.51 (1H, d, J=7.3), 7.58 (1H, t, J=8.5), 7.71 (2H, m), 8.68 (1H, s); ¹³C NMR (125 MHz, CDCl₃ or DMSO-d₆, rotamers present) 21.1, 39.7, 39.88, 39.9, 40.8, 46.9, 47.1, 55.7, 56.3, 57.6, 58.2, 60.4, 66.4, 67.5, 67.95, 67.98, 68.1, 68.2, 119.98, 119.99, 120.01, 124.93, 124.94, 125.05, 125.11, 127.07, 127.10, 127.13, 127.2, 127.7, 127.75, 127.79, 128.42, 127.44, 127.7, 128.8, 134.6, 141.2, 141.25, 141.34, 143.4, 143.5, 143.7, 143.8, 153.5, 156.0, 166.4, 172.0, 172.1, 176.3, 176.4; HRMS-ESI: m/z calcd for C₃₁H₂₇N₃O₈Na (M+Na)⁺ 592.1690, found 592.1702.

### General Procedure 2: Synthesis of Macrocycles

75-150 mg of 2-Cl Trityl chloride resin (0.912 mmol/g) was loaded with 0.138 M bromoacetic acid (1.2 equiv.) in DCM, with DIPEA (4 equiv.). This reaction was stirred for 1 h, after which time the resin was rinsed repeatedly with DCM. An amine in DMF (1.0 mL, 1 M) was added to the resin and allowed to react for 1 h, followed by repeated rinsing of the resin with DMF and DCM (3× each). A preactivated solution of a Q-Pro residue (3 equiv.) with HATU (3 equiv.) and DIPEA (6 equiv.) was prepared in 1.0 mL of DMF/DCM (1:1, anhydrous) 5 minutes prior to addition to the resin. This preactivated solution was added to the resin and allowed to react overnight. The resin was then rinsed and test-cleaved to check for complete addition to the resin. Fmoc deprotection was achieved with 2×5 min additions of 20% piperidine in DMF, followed by repeated rinsing with DMF and DCM. The next residues were added using standard peptoid coupling procedures, another Q-Pro addition (double coupling required), another peptoid coupling, and a final Q-Pro addition (double coupling required). After the final Q-Pro addition, the Fmoc was removed with 20% piperidine, followed by vigorous rinsing with DMF, and then DCM to drive off any base. The macrocycle precursors were cleaved from resin using 30% hexafluoroisopropanol in dichloromethane, purified by reversed-phase flash chromatography (5-95% ACN in Water, 0.1% formic acid modifier), and lyophilized.

The purified precursor was dissolved in anhydrous DCM (20 mL/mmol) with DIPEA (6 equiv.), and added to a stirred mixture of PyAOP (3 equiv.) in anhydrous DMF (30 mL/mmol) for a final solution concentration of 2 mM to promote intramolecular as opposed to intermolecular coupling. The reaction was stirred for 1 h, then checked via LCMS for completion. The macrocycle was diluted with ethyl acetate and washed with 3× saturated ammonium chloride, 3× saturated sodium bicarb, and 3× saturated sodium chloride, dried over sodium sulfate, and the solvent removed in vacuo. The resultant oil was purified via reversed-phase flash chromatography and lyophilized.

### Characterization Results

### QPM-1

Using general procedure 2, 88 mg of 2-Cl-Trityl Chloride Resin, 2-methoxyethyamine, and QPro-1.

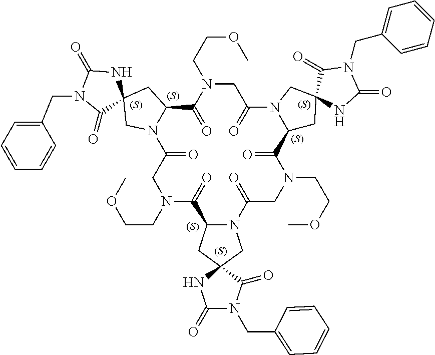

Recovered 63 mg pure (67% yield); ¹H NMR (500 MHz, CD3CN, 4 equiv. K-OTf) 2.16 (3H, dd, J=13.8, 1.8), 2.69 (3H, dd, J=13.9, 9.6), 3.29 (9H, m), 3.39-3.41 (3H, m), 3.43 (3H, m), 3.50-3.53 (3H, m), 3.59 (3H, dd, J=16.7), 3.75 (3H, m), 3.83 (3H, d, J=10.9), 3.96 (3H, d, J=10.9), 4.48 (3H, d, J=16.9), 4.63 (6H, m), 5.02 (3H, dd, J=9.5, 2.5), 6.79 (3H, s), 7.27-7.35 (15H, m); HRMS-ESI. m/z calcd for C₅₇H₆₆N₁₂O₁₅Na (M+Na)⁺ 1181.4463, found 1181.4676.

### QPM-2

Using general procedure 2, 90 mg of 2-Cl-Trityl Chloride Resin, 2-methoxyethyamine, QPro-1, QPro-2, and QPro-3.

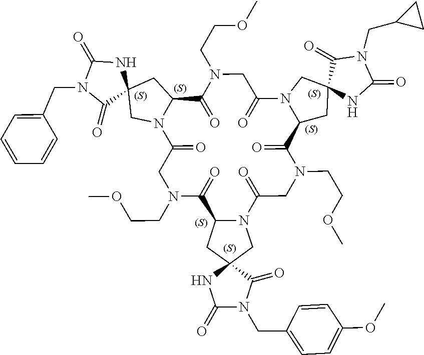

Recovered 56 mg pure (60% Yield); ¹H NMR (500 MHz, CD3CN, 4 equiv K-OTf) 0.28 (2H, m), 0.47 (2H, m), 1.08 (1H, m), 2.14 (3H, m), 2.68 (3H, m), 3.31 (11H, m), 3.42 (3H, m), 3.46 (3H, m), 3.53 (3H, m), 3.62 (3H, d, J=16.7), 3.75 (6H, s), 3.85 (3H, t, 10.2), 3.97 (3H, m), 4.55 (5H, m), 4.63 (2H, d, J=5.0), 5.02 (3H, d, J=9.5), 6.64 (1H, s), 6.72 (1H, s), 6.75 (1H, s), 6.87 (2H, m), 7.22 (2H, m), 7.31 (5H, m); HRMS-ESI. m/z calcd for C₅₅H₆₈N₁₂O₁₆Na (M+Na)⁺ 1175.4768, found 1175.4772.

### QPM-3

Using general procedure 2, 145 mg of 2-Cl-Trityl Chloride Resin, isobutylamine, and QPro-1.

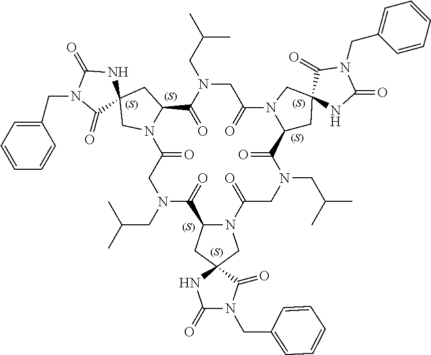

Recovered 101 mg (66% Yield); ¹H NMR (500 MHz, CD3CN, 4 equiv. K-OTf) 0.88 (9H, d, J=6.4) 0.95 (9H, d, J=6.4), 1.84 (3H, dt, J=13.8, 7.0), 2.10 (3H, d, J=14.0), 2.73 (3H, dd, J=13.7, 9.8), 2.95 (3H, dd, J=15.1, 8.7), 3.58 (3H, d, J=16.8), 3.62 (3H, m), 3.87 (3H, d, J=11.3), 3.99 (3H, d, J=11.0), 4.57 (3H, d, J=16.8), 4.63 (6H, m), 4.98 (3H, d, J=8.8), 6.82 (3H, s), 7.32 (15H, m); HRMS-ESI. m/z calcd for C₆₀H₇₂N₁₂O₁₂Na (M+Na)⁺ 1175.5285, found 1175.5315.

### QPM-4

Using general procedure 2, isobutylamine, QPro-1, QPro-2, and QPro-3.

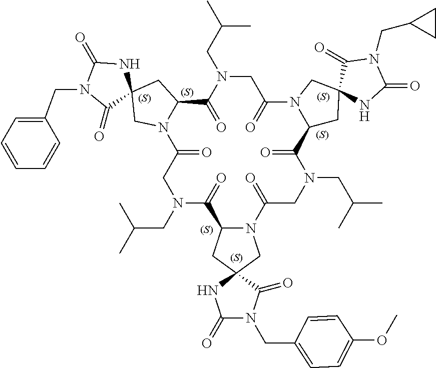

(60% Yield); ¹H NMR (500 MHz, CD3CN, 4 (equiv. K-OTf) 0.29 (2H, m), 0.47 (2H, m), 0.89 (9H, d, J=6.7), 0.96 (9H, d, J=6.7), 1.08 (1H, m), 1.86 (3H, m), 2.10 (3H, m), 2.73 (3H, m), 2.96 (3H, dd, J=15.0, 8.5), 3.3 (3H, m), 3.60 (2H, d, J=16.8), 3.61 (3H, m), 3.76 (3H, s), 3.87 (3H, m), 3.99 (3H, m), 4.55 (2H, m), 4.58 (3H, m), 4.62 (2H, m), 4.98 (3H, d, J=9.8), 6.72 (1H, s), 6.80 (1H, s), 6.83 (1H, s), 6.87 (2H, d, J=8.9), 7.23 (2H, d, J=8.9), 7.31 (5H, m); HRMS-ESI. m/z calcd for C₅₈H₇₄N₁₂O₁₃K (M+Na)⁺ 1185.5130, found 1185.5132.

### QPM-5

Using Step 1 of General Procedure 1, 30 mg of QPM-3, and tert-butyl bromoacetate. Purified by reversed phase flash chromatography (5-95% ACN in Water, 0.1% Formic Acid modifier).

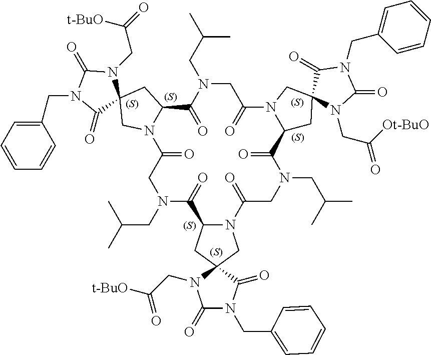

Recovered 31 mg (81% Step Yield; 53% Final Yield); ¹H NMR (500 MHz, CD3CN, 4 equiv K-OTf) 0.87 (9H, d, J=6.7) 0.95 (9H, d, J=6.4), 1.40 (27H, s), 1.85 (3H, m), 2.07 (3H, dd, J=13.4, 8.9), 2.56 (3H, dd, J=13.6, 8.1), 3.03 (3H, dd, J=15.0, 7.9), 3.51 (3H, dd, J=15.3, 6.1), 3.63 (3H, d, J=16.5), 3.80 (3H, m), 3.85 (3H, m), 3.97 (3H, m), 4.04 (3H, m), 4.35 (3H, d, J=16.8), 4.66 (6H, m), 4.93 (3H, t, J=8.4), 7.33 (15H, m); HRMS-ESI. m/z calcd for C₇₈H₁₀₂N₁₂O₁₈K (M+K)⁺ 1533.7067, found 1533.7043.

### QPM-6

Using Step 1 of General Procedure 1, 30 mg of QPM-3, and allyl bromide. Purified by reversed phase flash chromatography (5-95% ACN in Water, 0.1% Formic Acid modifier).

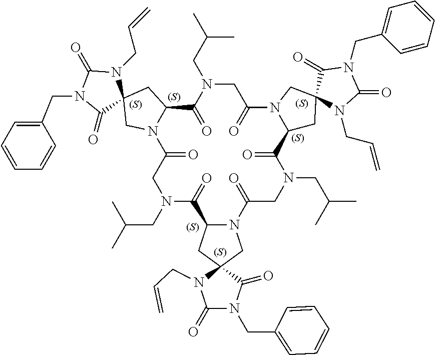

Recovered 27 mg (83% Step Yield; 55% Final Yield); ¹H NMR (500 MHz, CD3CN, 4 equiv K-OTf) 0.87 (9H, d, J=6.7) 0.95 (9H, d, J=6.7), 1.84 (3H, dt, J=13.8, 7.0), 2.17 (3H, dd, J=13.4, 8.9), 2.52 (3H, dd, J=13.4, 7.9), 3.02 (3H, dd, J=15.0, 8.2), 3.54 (3H, dd, J=14.5, 6.9), 3.60 (3H, d, J=16.8), 3.78 (3H, d, J=11.3), 3.93 (3H, d, J=11.1), 3.94 (3H, m), 4.05 (3H, m), 4.37 (3H, d, J=16.8), 4.63 (6H, m), 4.92 (3H, t, J=8.4), 5.18 (6H, m), 5.87 (3H, m), 7.33 (15H, m); HRMS-ESI m/z calcd for C₆₉H₈₄N₁₂O₁₂K (M+K)⁺ 1311.5963, found 1311.5975.

### QPM-7

Using general procedure 2, 85 mg of 2-Cl-Trityl Chloride Resin, isobutylamine, and QPro-4.

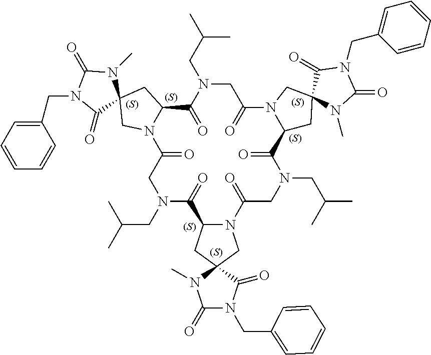

Recovered 64 mg pure (65%); ¹H NMR (500 MHz, CD3CN, 4 equiv K-OTf)¹H NMR (500 MHz, CD3CN, 4 equiv K-OTf) 0.88 (9H, d, J=6.7) 0.96 (9H, d, J=6.7), 1.84 (3H, dt, J=13.8, 7.0), 2.15 (3H, dd, J=13.4, 8.2), 2.50 (3H, dd, J=13.4, 8.4), 2.88 (9H, s), 3.04 (3H, dd, J=15.0, 8.2), 3.53 (3H, dd, J=14.2, 6.6), 3.63 (3H, 16.8), 3.75 (3H, d, J=11.3), 3.95 (3H, d, J=11.0), 4.41 (3H, d, J=16.8), 4.62 (6H, m), 4.94 (3H, t, J=8.4), 7.33 (15H, m); HRMS-ESI: m/z calcd for C₆₃H₇₈N₁₂O₁₂Na (M+Na)⁺ 1217.5754, found 1217.5795.

### QPM-8

Using general procedure 2, 87 mg of 2-Cl-Trityl Chloride Resin isobutylamine, and the enantiomer of QPro-1.

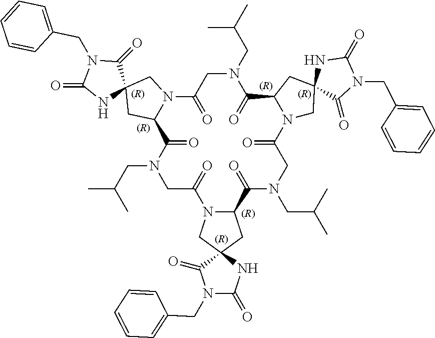

Recovered 58 mg pure (62% yield); ¹H NMR (500 MHz, CD3CN, 4 equiv K-OTf) 0.91 (9H, d, J=6.7) 1.01 (9H, d, J=6.7), 1.84 (3H, m), 2.17 (3H, m), 2.81 (3H, dd, J=13.9, 9.9), 3.13 (3H, dd, J=14.8, 9.3), 3.25 (3H, dd, J=15, 5.2), 3.63 (3H, d, J=16.8), 3.86 (3H, d, J=11.3), 4.08 (3H, m), 4.49 (3H, d, J=16.8), 4.62 (6H, m), 4.93 (3H, d, J=8.9), 6.57 (3H, s), 7.32 (15H, m); HRMS-ESI: m/z calcd for C₆₀H₇₂N₁₂O₁₂Na (M+Na)⁺ 1175.5285, found 1175.5303.

### Experimental Details of Single Crystal X-Ray Diffraction

Single crystals of C₆₄H₈₀N₁₂O₁₄ (QPM-3) were selected and collected on a synchrotron diffractometer with an Eiger 16 MPixel detector at the NSLS-II, 17-ID-2, Brookhaven National Lab. The crystal was kept at 100(2) K during data collection. Using ShelXS, the structure was solved with dual space methods, and refined using ShelXL refinement package within Olex2 as a GUI.

Crystal Data for C₆₄H₈₀N₁₂O₁₄ (M=1241.40 g/mol): triclinic, space group P-1 (no. 2), a=11.642(2) Å, b=18.450(4) Å, c=32.122(6) Å, a=104.72(3)°, β=94.74(3)°, γ=105.21(3)°, V=6356(3) Å³, Z=4, T=293(2) K, μ(synchrotron)=0.087 mm⁻¹, Dcalc=1.297 g/cm³, 12333 reflections measured (2.928°≤2Θ≤33.364°), 6839 unique (Rᵢₙₜ=0.0557, R_{sigma}=0.0935) which were used in all calculations. The final R₁ was 0.0874 (I>2σ(I)) and wR₂ was 0.2549 (all data).

Number of restraints—462, number of constraints—0.

### Details:

```
1. Fixed Uiso
  At 1.2 times of:
  All C(H) groups, All C(H,H) groups, All C(H,H,H,H) groups, All N(H)
groups
  At 1.5 times of:
  All C(H,H,H) groups
2. Rigid bond restraints
  C22, C23, C24, C25, C26, C27, C28, C0AA, C1AA, C2AA, C3AA, C4AA, C5AA
  with sigma for 1-2 distances of 0.005 and sigma for 1-3 distances of
0.005
  C25A, C24A, C24B, C23B, C28A, C28B, C27A, C27B, C23A, C25B, C26A,
C26B, C22A
  with sigma for 1-2 distances of 0.01 and sigma for 1-3 distances of
0.01
  C1S, C1T
  with sigma for 1-2 distances of 0.005 and sigma for 1-3 distances of
0.005
3. Uiso/Uaniso restraints and constraints
C22 ≈ C23 ≈ C24 ≈ C25 ≈ C26 ≈ C27 ≈ C28 ≈ C0AA
≈ C1AA ≈ C2AA ≈ C3AA ≈ C4AA ≈ C5AA: within 2A with sigma of
0.005 and sigma for terminal atoms of 0.01
C22A ≈ C23B ≈ C23A ≈ C28A ≈ C28B ≈ C24B ≈ C24A ≈
C25A ≈ C25B ≈ C26A ≈ C26B ≈ C27A ≈ C27B: within 2A with
sigma of 0.04 and sigma for terminal atoms of 0.08
C1S ≈ C1T: within 2A with sigma of 0.005 and sigma for terminal atoms
of
0.01
4. Others
  Sof(H22G)=Sof(H22H)=Sof(C0AA)=Sof(C1AA)=Sof(H1AC)=Sof(C2AA)=Sof(H2AA)=
  Sof(C3AA)=Sof(H3AA)=Sof(C4AA)=Sof(H4AA)=Sof(C5AA)=Sof(H5AC)=1-FVAR(1)
Sof(H22E)=Sof(H22F)=Sof(C23)=Sof(C24)=Sof(H24)=Sof(C25)=Sof(H25)=Sof(C2
6) =
  Sof(H26)=Sof(C27)=Sof(H27)=Sof(C28)=Sof(H28)=FVAR(1)
  Sof(H22C)=Sof(H22D)=Sof(C23B)=Sof(C24B)=Sof(H24B)=Sof(C25B)=Sof(H25B)=
  Sof(C26B)=Sof(H26B)=Sof(C27B)=Sof(H27B)=Sof(C28B)=Sof(H28B)=1-FVAR(2)
  Sof(H22A)=Sof(H22B)=Sof(C23A)=Sof(C24A)=Sof(H24A)=Sof(C25A)=Sof(H25A)=
  Sof(C26A)=Sof(H26A)=Sof(C27A)=Sof(H27A)=Sof(C28A)=Sof(H28A)=FVAR(2)
  Sof(H2SC)=Sof(H2SD)=Sof(C1T)=Sof(H1TA)=Sof(H1TB)=Sof(H1TC)=1-FVAR(3)
  Sof(C1S)=Sof(H1SA)=Sof(H1SB)=Sof(H1SC)=Sof(H2SA)=Sof(H2SB)=FVAR(3)
5.a Ternary CH refined with riding coordinates:
  C3A(H3A), C7A(H7A), C11A(H11A), C14A(H14A), C30A(H30A), C46A(H46A),
C3(H3),
  C7(H7), C11(H11), C14(H14), C30(H30), C46(H46)
5.b Secondary CH2 refined with riding coordinates:
  C1A(H1AA,H1AB), C5A(H5AA,H5AB), C9A(H9AA,H9AB), C13A(H13A,H13B),
C17A(H17A,
  H17B), C18A(H18A,H18B), C22A(H22A,H22B), C22A(H22C,H22D),
C29A(H29A,H29B),
  C33A(H33A,H33B), C34A(H34A,H34B), C38A(H38A,H38B), C45A(H45A,H45B),
C49A(H49A,
  H49B), C50A(H50A,H50B), C54A(H54A,H54B), C1(H1A,H1B), C5(H5A,H5B),
C9(H9A,H9B),
  C13(H13C,H13D), C17(H17C,H17D), C18(H18C,H18D), C22(H22E, H22F),
C22(H22G,
  H22H), C29(H29C,H29D), C33(H33C,H33D), C34(H34C,H34D), C38(H38C,H38D),
  C45(H45C,H45D), C49(H49C,H49D), C50(H50C,H50D), C54(H54C,H54D),
C6S(H6SA,H6SB),
  C2S(H2SA,H2SB), C2S(H2SC,H2SD)
5.c Me refined with riding coordinates:
  C15A(H15A,H15B,H15C), C16A(H16A,H16B,H16C), C31A(H31A,H31B,H31C),
C32A(H32A,
  H32B,H32C), C47A(H47A,H47B,H47C), C48A(H48A,H48B,H48C),
C15(H15D,H15E,H15F),
  C16(H16D,H16E,H16F), C31(H31D,H31E,H31F), C32(H32D,H32E,H32F),
C47(H47D,H47E,
  H47F), C48(H48D,H48E,H48F), C5S(H5SA,H5SB,H5SC), C1S(H1SA,H1SB,H1SC),
C1T(H1TA,
  H1TB,H1TC)
5.d Aromatic/amide H refined with riding coordinates:
  N21A(H21A), N41A(H41A), N61A(H61A), C24A(H24A), C25A(H25A),
C26A(H26A),
  C27A(H27A), C28A(H28A), C40A(H40A), C41A(H41B), C42A(H42A),
C43A(H43A),
  C44A(H44A), C56A(H56A), C57A(H57A), C58A(H58A), C59A(H59A),
C60A(H60A),
  N21(H21), N41(H41), N61(H61), C24(H24), C25(H25), C26(H26), C27(H27),
C28(H28),
  C40(H40), C41(H41C), C42(H42), C43(H43), C44(H44), C56(H56), C57(H57),
  C58(H58), C59(H59), C60(H60), C1AA(H1AC), C2AA(H2AA), C3AA(H3AA),
C4AA(H4AA),
  C5AA(H5AC), C24B(H24B), C25B(H25B), C26B(H26B), C27B(H27B), C28B(H28B)
5.e Fitted hexagon refined as free rotating group:
  C23A(C24A,C25A,C26A,C27A,C28A), C23(C24,C25,C26,C27,C28),
C0AA(C1AA,C2AA,C3AA,
  C4AA,C5AA), C23B(C24B,C25B,C26B,C27B,C28B)
5.f Idealised Me refined as rotating group:
  C8S(H8SA,H8SB,H8SC), C4S(H4SA,H4SB,H4SC)
```

This report has been created with Olex2, compiled on 2018.05.29 svn.r3508 for OlexSys.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound or salt thereof having the structure of Formula (I) 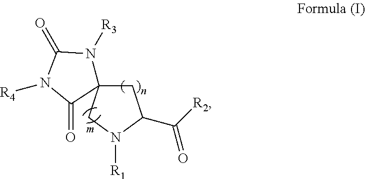
wherein R₁ is H or a protecting group;
wherein each R₂, R₃, and R₄ is independently selected from the group consisting of H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted —Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural amino acid, unnatural amino acid, and 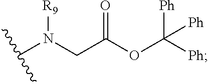
wherein Y is selected from the group consisting of C, N, O, S, and P;
wherein R₅, R₆, and R₇ are independently selected from the group consisting of H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, and sulfoxy;
wherein o is an integer represented by 0, 1, or 2; and
wherein p is an integer represented by 0, 1, or 2;
wherein m is an integer represented by 1 or 2;
wherein n is an integer represented by 1 or 2; and
wherein the compound is an unnatural amino acid.

2. The compound of claim 1, wherein the protecting group is a carbonyl protecting group, carbamate protecting group, sulfonamide protecting group, trityl protecting group, 1-(4,4-dimethyl-2,6-dioxocyclohex-1-ylidene)-3-ethyl (Dde) protecting group, or 1-(4,4-dimethyl-2,6-dioxocyclohex-1-ylidene)-3-methylbutyl (ivDde) protecting group.

3. The compound of claim 2, wherein the carbonyl protecting group is selected from the group consisting of a methoxycarbonyl protecting group, tert-butoxycarbonyl protecting group (BOC group), benzyloxycarbonyl protecting group,

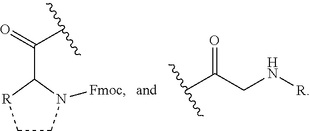

4. The compound of claim 3, wherein the methoxycarbonyl protecting group is 9-fluorenyl methoxycarbonyl (Fmoc)

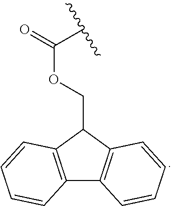 [SMILES: *C(=O)OCC1c2ccccc2-c2ccccc21]

5. The compound of claim 1,
wherein R₁ is a protecting group; R₂ is hydroxyl, hydroxyalkyl, hydroxyalkenyl, hydroxyalkynyl, or hydroxyaryl; m is an integer represented by 1; and n is an integer represented by 1;
wherein R₁ is a protecting group; R₂ is hydroxyl, hydroxyalkyl, hydroxyalkenyl, hydroxyalkynyl, or hydroxyaryl; m is an integer represented by 2; and n is an integer represented by 1; or
wherein R₁ is a protecting group; R₂ is hydroxyl, hydroxyalkyl, hydroxyalkenyl, hydroxyalkynyl, or hydroxyaryl; m is an integer represented by 1; and n is an integer represented by 2.

6. The compound of claim 5, wherein the compound comprises proline.

7. The compound of claim 1, wherein the compound is selected from the group consisting of: 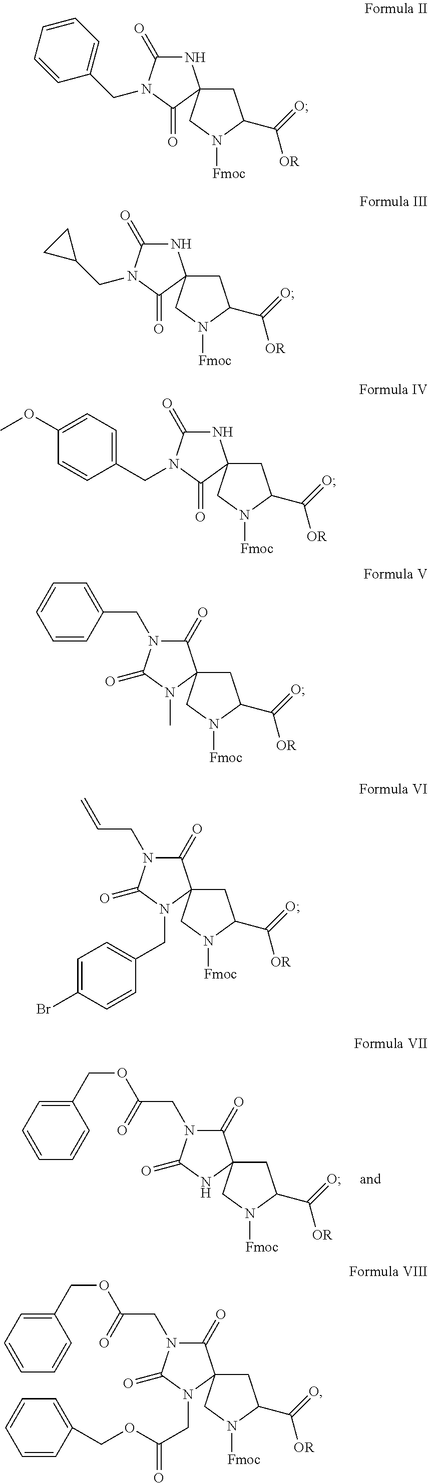
wherein R is selected from the group consisting of H, alkyl, and a protecting group.

8. The compound of claim 1, wherein the compound comprises at least two stereocenters.

9. The compound of claim 8, wherein the compound is selected from the group consisting of: 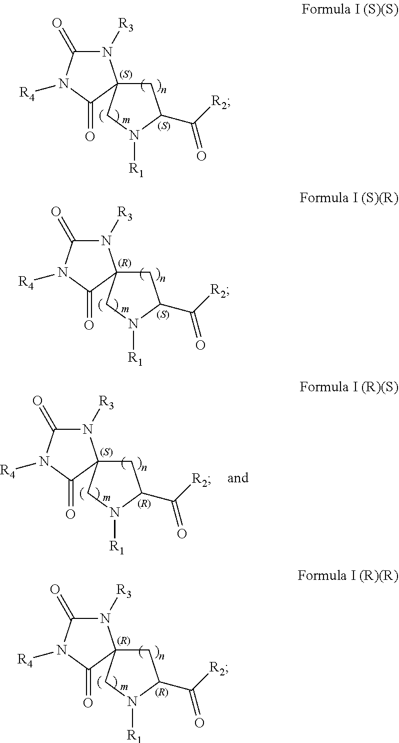
wherein R₁ is H or a protecting group;
wherein each R₂, R₃, and R₄ is independently selected from the group consisting of H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted —Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural amino acid, unnatural amino acid, and 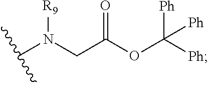
wherein Y is selected from the group consisting of C, N, O, S, and P;
wherein R₅, R₆, and R₇ are independently selected from the group consisting of H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, and sulfoxy;
wherein o is an integer represented by 0, 1, or 2; and
wherein p is an integer represented by 0, 1, or 2;
wherein m is an integer represented by 1 or 2; and
wherein n is an integer represented by 1 or 2.

10. The compound of claim 8, wherein the compound is selected from the group consisting of:

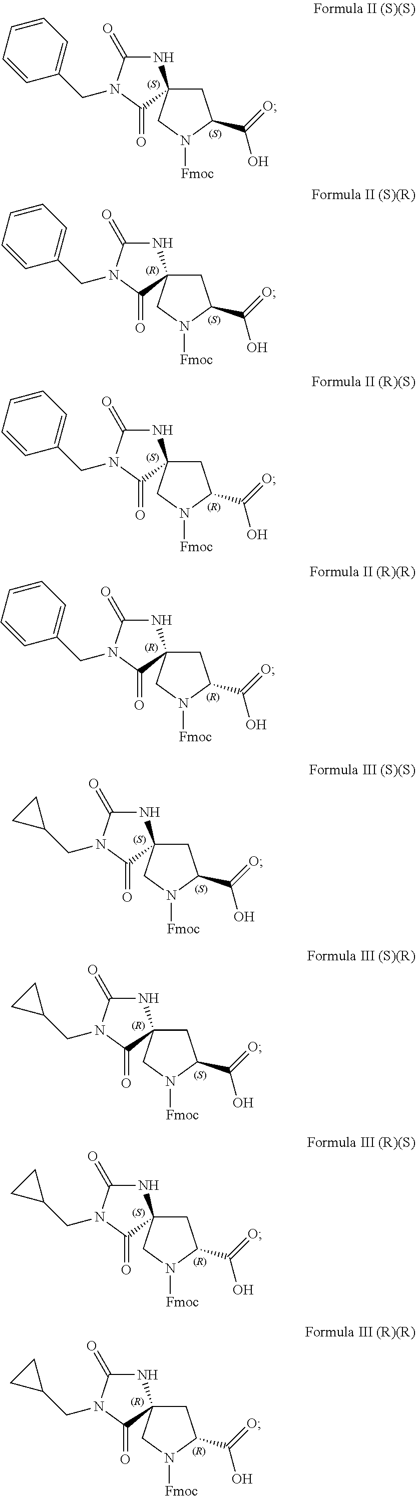

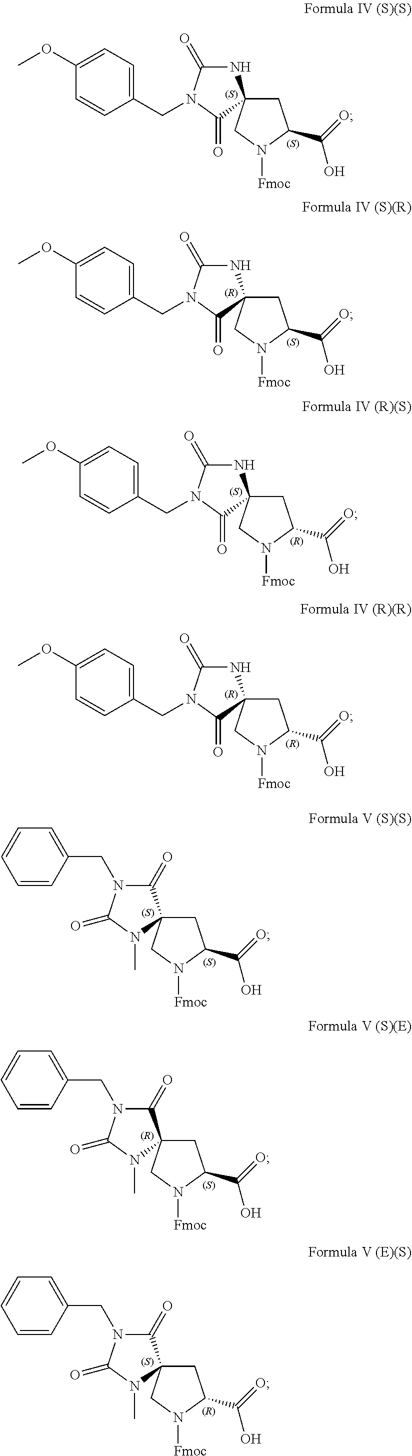

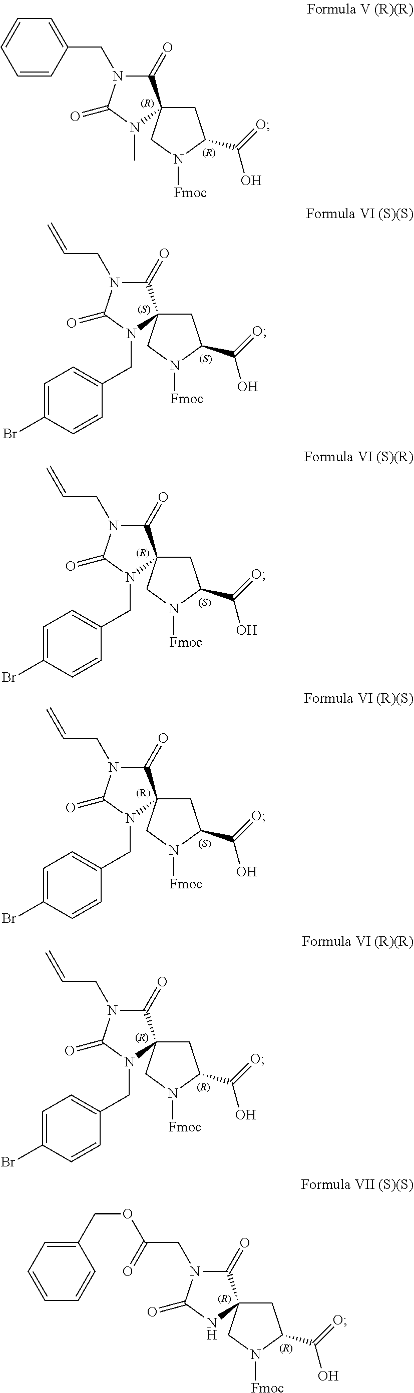

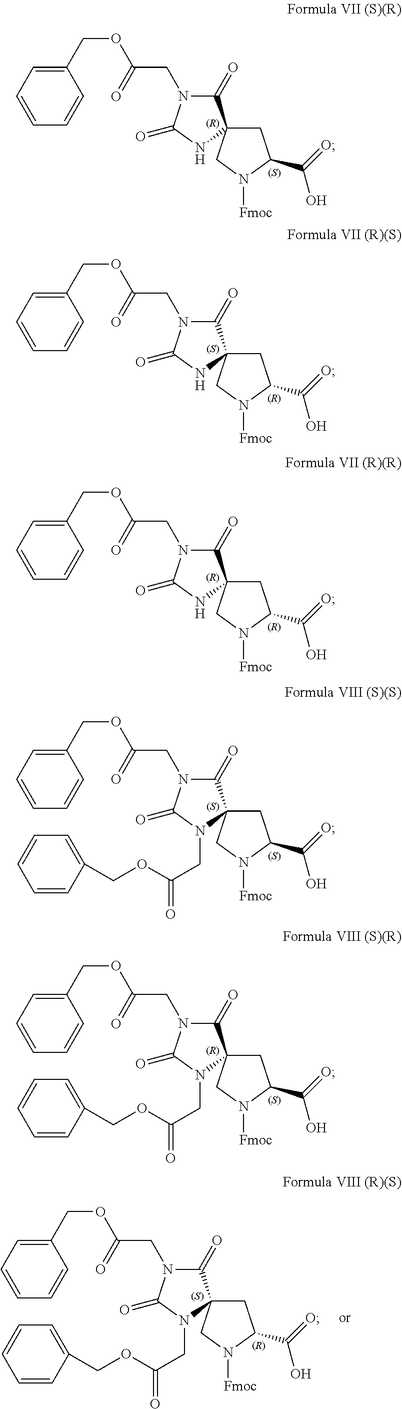

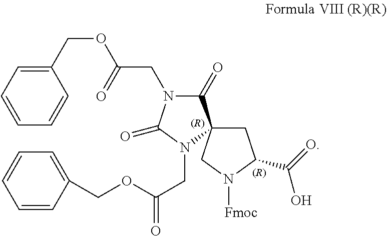

11. An amino acid sequence comprising one or more compounds of claim 1.

12. The amino acid sequence of claim 11, wherein the amino acid sequence is selected from the group consisting of a peptide or a fragment thereof, polypeptide or a fragment thereof, and protein or a fragment thereof.

13. A foldamer comprising one or more compounds of claim 1.

14. The foldamer of claim 13, wherein the foldamer is selected from the group consisting of a peptidomimetic foldamer, peptide, bispeptide, β-peptide, γ-peptide, δ-peptide, nucleotidomimetic foldamer, abiotic foldamer, peptoid, aedamer, aromatic oligamide foldamer, spiroligomer, arylamine foldamer, and chiral oligomers of pentenoic amides (COPAs).

15. A macrocycle comprising one or more compounds of claim 1.

16. The macrocycle of claim 15, wherein the macrocycle has the structure of Formula (IX) 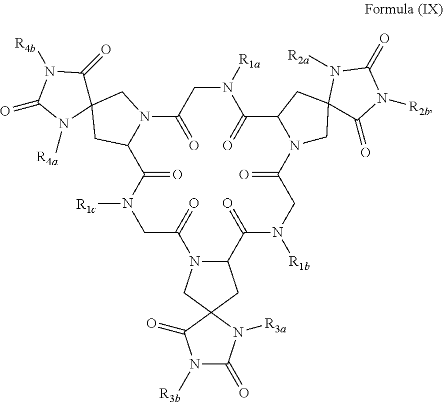
wherein R₁ₐ, R_{1b}, R_{1c}, R₂ₐ, R_{2b}, R₃ₐ, R_{3b}, R₄ₐ, and R_{4b} are independently selected from the group consisting of H, halogen, alkyl, substituted alkyl, cycloalkyl, substituted cycloalkyl, —Y(R₅)ₒ(R₆)ₚ-cycloalkyl, substituted —Y(R₅)ₒ(R₆)ₚ-cycloalkyl, heterocycloalkyl, substituted heterocycloalkyl, —Y(R₅)ₒ(R₆)ₚ-heterocycloalkyl, substituted —Y(R₅)ₒ(R₆)ₚ-heterocycloalkyl, alkenyl, substituted alkenyl, cycloalkenyl, substituted cycloalkenyl, —Y(R₅)ₒ(R₆)ₚ-cycloalkenyl, substituted —Y(R₅)ₒ(R₆)ₚ-cycloalkenyl, alkynyl, substituted alkynyl, cycloalkynyl, substituted cycloalkynyl, —Y(R₅)ₒ(R₆)ₚ-cycloalkynyl, substituted —Y(R₅)ₒ(R₆)ₚ-cycloalkynyl, aryl, substituted aryl, —Y(R₅)ₒ(R₆)ₚ-aryl, substituted —Y(R₅)ₒ(R₆)ₚ-aryl, heteroaryl, substituted heteroaryl, —Y(R₅)ₒ(R₆)ₚ-heteroaryl, substituted —Y(R₅)ₒ(R₆)ₚ-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R₅)ₒ(R₆)ₚ-ester, —Y(R₅)ₒ(R₆)ₚ, ═O, —NO₂, —CN, sulfoxy, secondary amide, tertiary amide, CON—R₇ amide, natural aminoacids, unnatural amino acid, and 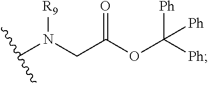
wherein Y is selected from the group consisting of C, N, O, S, and P;
wherein R₅, R₆, and R₇ are independently selected from the group consisting of H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, and sulfoxy;
wherein o is an integer represented by 0, 1, or 2; and
wherein p is an integer represented by 0, 1, or 2.

17. The macrocycle of claim 15, wherein the macrocycle is selected from a group consisting of

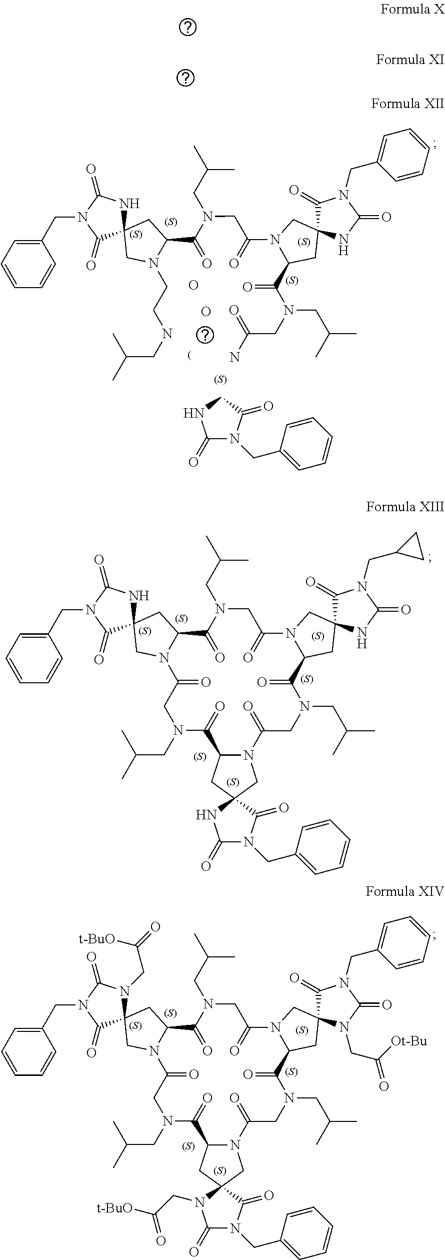

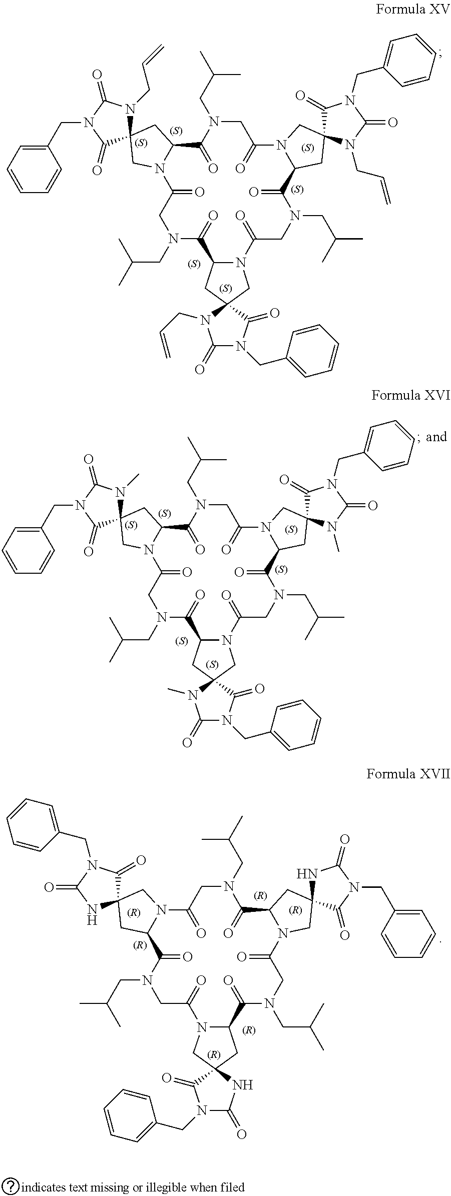

18. The macrocycle of claim 15, further comprising at least one metal ion.

19. The macrocycle of claim 18, wherein the metal ion is a metal cation.

20. The macrocycle of claim 19, wherein the metal cation is a Li⁺, Na⁺, or K⁺.

21. A composition comprising one or more compounds of claim 1.

22. A method of mimicking a natural amino acid in a subject in need thereof, the method comprising administering to the subject a therapeutically effective amount of one or more compound of claim 1 or a composition thereof,
wherein the compound of claim 1 mimics the natural amino acid.

23. The method of claim 22, wherein the compound of claim 1 mimics the function of the natural amino acid.

24. The method of claim 22, wherein the compound of claim 1 mimics the structure of the natural amino acid.
